(19) United States
(12) Patent Application Publication
(10) Pub. No.: US 2004/0009530 A1
(43) Pub. Date: Jan. 15, 2004

(54) Engineered binding proteins

(76) Inventors: David S. Wilson, Mountain View, CA (US); Steffen Nock, Redwood City, CA (US)

Correspondence Address: Pennie & Edmonds, LLP, 3300 Hillview Avenue, Palo Alto, CA, 94304, US

(21) Appl. No.: 10/347,542

(22) Filed: Jan. 16, 2003

Related U.S. Application Data
(60) Provisional application No. 60/349,999, filed on Jan. 17, 2002.
(60) Provisional application No. 60/349,804, filed on Jan. 16, 2002.

(51) Int. Cl. G01N 33/53; C07K 14/00

(52) U.S. Cl. 435/7.1; 530/350

(57) ABSTRACT

Engineered binding proteins are provided. In some cases, the parent protein corresponding to the engineered protein has a three-layer swiveling β/β/α domain. In other cases, the parent protein corresponding to the engineered protein has a rubredoxin-like fold. At least one portion of the primary sequence of the engineered protein is determined by an engineering scheme. In some case, the engineered protein is characterized by an ability to bind to a compound that the parent protein does not bind. In some cases, the parent protein is derived from a domain of a chaperonin or a rubredoxin. One form of engineering scheme used is a randomization scheme. A method for making libraries of engineered proteins, all based on a single parent protein is provided. Methods to identify proteins that bind to compounds of interest in libraries of engineered libraries is provided. An array of engineered proteins immobilized on a support is provided. Each engineered protein in the array is a chaperonin domain or a rubredoxin that has been subjected to an engineering scheme.

### 1. CROSS-REFERENCE TO RELATED APPLICATIONS

This application claims priority, under 35 U.S.C. § 119(e), to U.S. Provisional Patent Application No. 60/349,999, filed Jan. 17, 2002, which is incorporated herein by reference in its entirety. Furthermore, this application claims priority, under 35 U.S.C. § 119(e), to U.S. Provisional Patent Application No. 60/349,804, filed on Jan. 16, 2002, which is incorporated herein, by reference, in its entirety.

### 2. FIELD OF THE INVENTION

The present invention relates to engineered binding protein libraries that are derived from chaperonin or rubredoxin.

### 3. BACKGROUND OF THE INVENTION

Proteins having relatively stable three-dimensional structures may be used as reagents for the design of engineered products. One method for exploiting such proteins relies on the assignment of the different regions of a protein or protein domain of known structure into two different categories, the scaffold region and one or more diversifiable regions. The scaffold region is the portion of the protein that is largely responsible for conferring global three-dimensional structure (the “fold”). A diversifiable region is less critical to conferring the global three-dimensional structure of the protein, and may even be incidental to conferring or maintaining such structure. Diversifiable regions are generally surface exposed turns and loops. A diversifiable region is therefore amenable to engineering techniques that alter the native sequence of such regions. In the case of such engineering, the parent protein is referred to as the parent protein, and the altered protein is referred to as the “engineered protein” or “engineered domain”. This alteration (engineering) can be of a random nature, and can result in a large collection of different polypeptide sequences in place of the corresponding sequence in the parent protein. The resulting collection of proteins is called a “protein library.” A protein library can be based on the randomization of a single diversifiable region or of a plurality of diversifiable regions. A diversifiable region that is engineered to create a collection of different sequences, all in the context of the same protein scaffold, is referred to as a “diversified region.” It is the object of the randomization scheme that the majority of the engineered proteins in the library maintain the overall three-dimensional structure as the parent protein. There are three main advantages to having engineered proteins that retain the structure of the parent protein: (i) increased stability against proteases, (ii) increased solubility, and (iii) increased structural order (decreased chain entropy). By contrast, engineered proteins that do not maintain the overall structure of the parent protein and are, rather, unstructured polypeptides, are unstable to proteases, have poor solubility, and generally do not bind tightly to compounds due to the large increase in the order of the polypeptide chain that must occur upon binding to the compound; this increased order (decreased entropy) has a significant energetic cost associated with it, and therefore lowers the affinity of the interaction between the engineered protein and the compound.

An engineered protein can contain one or more diversifiable regions, and one or more diversified regions. After a library of proteins, with one of more diversified regions, is produced, members of this library with desirable properties can be identified by selection or by screening, or through a combination of selection and screening.

Natural antibodies include a scaffold region and diversified regions. Antibodies have the same protein fold due to conservation of the scaffold region in such proteins. The diversified regions in antibodies are called complementarity-determining regions (CDR), and consist of six surface loops or turns, all located on one face of the antitbody antigen-binding domain. In the immune system, specific antibodies that bind to foreign compounds (antigens), such as foreign proteins, are selected and amplified from a large library. The process can be reproduced in vitro using combinatorial library techniques. The successful display of chains of antibody fragments on the surface of bacteriophage has made it possible to generate a large number of antibodies with different CDRs, and to subsequently identify antibodies from this library that bind to proteins of interest, using a selection technique called phage display (McCafferty et al., 1990, Nature 348, pp. 552-554; Barbas et al., 1991, Proc. Natl. Acad. Sci. USA 88, pp. 7978-7982; and Winter et al., 1994, Annu. Rev. Immunol. 12, pp. 433-455. The use of antibodies in commercial applications, however, has certain disadvantages. First, antibodies are complex multimeric molecules that include disulfide bonds. As a result, antibodies are sensitive to a number of environmental conditions such as reduction. This sensitivity limits the expression systems that can be used for producing antibodies. In vitro protein expression systems as well as in vivo systems for cytoplasmic protein expression result in proteins being synthesized under reducing conditions. The sensitivity to reduction also limits the utility of the binding proteins once they have been produced. Several types of bioconjugation reactions, which are required to attach labels to proteins, to attach proteins to surfaces, etc., require a reduction step for the synthesis. Second, antibodies typically have poor expression profiles and poor solubility. Furthermore, antibodies are difficult to refold. Finally, antibodies are very large. All of these problems make the commercial use of antibodies as protein scaffold libraries, unsatisfactory.

Because of the disadvantages of antibodies, a number of workers have developed binding agents with alternative structural scaffolds. For example, a “minibody” scaffold has been designed by deleting three beta strands from a heavy chain variable domain of a monoclonal antibody (Tramontano et al., 1994, J. Mol. Recognit. 7:9; and Martin et al., 1994, The EMBO Journal 13, pp. 5303-5309). This protein includes 61 residues and can be used to present two hypervariable loops. These two loops have been randomized to create diversified regions. Libraries of proteins based on this diversification scheme have undergone selection using phage display, allowing for the identification of engineered proteins that bind to proteins of interest. Thus far, however, engineered proteins with this scaffold appear to have somewhat limited utility due to solubility problems.

Another scaffold used for engineering is derived from tendamistatin, a 74 residue, six-strand beta sheet sandwich held together by two disulfide bonds (McConnell and Hoess, 1995, J. Mol. Biol. 250:460). This parent protein includes three loops, but, to date, only two of these loops have been examined for randomization potential. One disadvantage with tendamistatin is that it includes a disulfide bond that is not stable under reducing conditions. Many binding protein commercial applications require the binding proteins to be durable and highly resistant to environmental variables such as reducing conditions. Therefore, the use of tendamistatin in the commercial setting is problematic.

In another approach, scaffolds are derived from V-like domains (Coia et al. WO 99/45110). V-like domains refer to a domain that has similar structural features to the variable heavy (VH) or variable light (VL) domains of antibodies. The approach of Coia et al. has the same drawbacks as tendamistatin because the V-like domains of Coia et al. have disulfide bonds, which are not stable under reducing conditions. In the approach of Desmet et al., a β-sandwich structure derived from the naturally occurring extracellular domain of CTLA-4 is used as a scaffold (See Desmet et al. WO 00/60070). Like the scaffolds of Coia et al., those based on CTLA-4 include disulfide bridges and are therefore not stable under the reducing conditions that may arise in the commercial use of engineered binding proteins.

In yet another approach, workers have used scaffolds based on the fibronectin type III domain or related fibronectin-like proteins. The overall fold of the fibronectin type III (Fn3) domain is closely related to that of the smallest functional antibody fragment, the variable region of the antibody heavy chain. The overall fold of the 10^{th} type III domain of human fibronectin is illustrated in FIG. 1. Fn3 is best described as a β-sandwich similar to that of the antibody VH domain, except that Fn3 has seven β-strands instead of nine. There are three loops at the end of Fn3; the positions of BC, DE and FG loops (FIG. 1B) approximately correspond to those of CDR1, 2 and 3 of the VH domain of an antibody. Fn3 is advantageous because it does not have disulfide bonds. Therefore, Fn3 is stable under reducing conditions, unlike antibodies and their fragments (see Koide PCT WO 98/56915; Lipovsek and Wagner PCT WO 01/64942; Lipovsek PCT WO 00/34784). A protein library was created in which one or more of the surface-exposed loops (AB, BC, CD, DE, EF, and FG) of the Fn3 domain was diversified using a randomization scheme.

A significant drawback with the fibronectin scaffold is revealed by examination of FIG. 1. FIG. 1 shows that the N-terminus of Fn3 is proximate to the BC, DE and FG loops while the C-terminus of Fn3 is proximate to the AB, CD, and EF loops. This is disadvantageous for certain commercial uses of protein-binding agents where it is desirable to attach the binding proteins to a chip or other immobilization surface so that arrays of binding proteins, each having binding affinity to a protein of interest, may be prepared. This is because it is often beneficial to attach proteins to surfaces at or near the N-terminus or C-terminus of the proteins. Yet, N-terminal attachment of engineered proteins with the Fn3 scaffold to a surface could mask the BC, DE and FG loops because the N-terminus is on the same face as these loops. As a result, it is likely that N-terminal attachment of an Fn3 domain in which the BC, DE and FG loops have been engineered will interfere with the binding ability of the binding protein. Furthermore, C-terminal attachment of the binding proteins with the Fn3 scaffold to a surface will potentially mask the AB, CD, and EF loops. Thus, it is likely that C-terminal attachment of an Fn3 domain in which the AB, CD, and EF loops are randomized will interfere with the binding ability of the engineered proteins. The placement of the termini of the protein domains with respect to the diversifiable regions is also important for other applications. The methods used for the selection of binding proteins from protein libraries, such as phage display, microbial display, ribosome display, mRNA display, and peptide-on-plasmid display, all require attachment of one of the termini of the library proteins to the genetic encoding unit (phage, microbe, ribosome, mRNA or plasmid). Thus, it is advantageous if the termini are distal from the diversifiable regions, because the binding activity of these regions may be masked by the genetic encoding unit if it is structurally adjacent to them. Similarly, pharmaceutical applications of binding proteins generally require them to be derivatized with a carrying agent, such as poly(ethyleneglycol), and this is frequently accomplished by placing the carrying agent at or near one of the termini.

A number of other workers in the field have developed binding agents using the scaffold approach. For a review, see Smith, 1998, TIBS 23, pp. 457-460; Doi and Yanagawa, 1998, Cell. Mol. Life Sci. 54, 394-404; and Nyrgren and Uhlén, 1997, Current Opinion in Structural Biology. However, the development of an ideal scaffolding system necessitates optimization of a considerable number of variables, such as protein expression, protein solubility, and protein stability. In addition, such parent proteins must have a sufficient number and positioning of diversifiable regions to be productively exploited using diversification techniques, without causing disruption of the overall scaffold fold. Furthermore, some applications require protein-binding agents that can withstand derivatization so as to be bound to a chip, slide or bead.

Accordingly, given the above background, despite much work in the field, a need remains in the art for the development of additional systems for producing protein-binding agents based on the scaffold concept.

### 4. SUMMARY OF THE INVENTION

The present invention provides commercially useful protein scaffolds that have a number of advantageous applications. In particular, the scaffolds of the present invention may be used to generate libraries of engineered proteins with desirable physical and chemical characteristics, such as stability and solubility. A library of engineered proteins may be used to select and screen for members that have binding affinity to compounds of interest. Furthermore, the individual members of these libraries that have affinity to proteins of interest may be attached to fixed surfaces, such as addressable chips, in order to provide an array of engineered proteins with predetermined binding affinity. Advantageously, in one embodiment, the engineered proteins of the present invention are attached to fixed surfaces using either N-terminal or C-terminal chemistries. In one embodiment, the engineered proteins of the present invention are not stabilized by disulfide bridges. Because of this, the engineered proteins are generally stable under reducing conditions. In one embodiment, protein scaffolds are selected from proteins of known structure from organisms that are tolerant of exceedingly high temperatures. Proteins selected from such organisms have unusual thermal stability. This thermal stability is advantageously retained in libraries of engineered proteins that are produced based upon such scaffolds.

### 4.1 Engineered Three-Layer Swiveling Beta/Beta/Alpha Proteins

A first aspect of the present invention provides an engineered protein. The engineered protein is based on a parent protein, but mutagenized that maintains the overall global three-dimensional structure (fold) of the parent protein by leaving unchanged the region of the parent protein that is largely responsible for maintaining that fold. The region of a parent protein that is largely responsible for conferring the three-dimensional structure on that protein or on related engineered proteins is referred to as the scaffold. The scaffold may be continuous or discontinuous in three-dimensional space, and is generally discontinuous with respect to the linear amino acid sequence of the protein. Nevertheless, for any particular protein, this region and (the scaffold) is referred to in herein in the singular.

In one embodiment, the parent protein corresponding to the engineered protein has a three-layer swiveling β/β/α domain in which the central beta sheet is parallel and the other beta sheet is antiparallel. The engineered protein corresponding to the parent protein is made by subjecting the parent protein to an engineering scheme. In some instances, this engineering scheme comprises randomizing portions of the parent protein. Another embodiment provides an engineered protein in which the parent protein that corresponds to the engineered protein comprises a three-layer swiveling β/β/α domain. The central beta sheet of the three-layer swiveling β/β/α domain is parallel and the other beta sheet in the three-layer swiveling β/β/α domain is antiparallel. In this embodiment, at least one portion of the primary sequence of the engineered protein is determined by an operation of an engineering scheme on the primary sequence of the parent protein. However, the total length of the at least one portion of the primary sequence of the engineered protein is constrained so that it does not exceed fifty percent of the length of the primary sequence of the engineered protein. Further, the total length of the at least one portion of the primary sequence that is subjected to an engineering scheme comprises at least five percent of the length of the primary sequence of the engineered protein.

In some embodiments, the engineered protein is characterized by its ability to bind to a compound that the corresponding parent protein does not specifically bind. In some embodiments, the three-layer swiveling β/β/α domain of the parent protein has a β-sandwich architecture comprising a first β sheet and a second β sheet in which the first β sheet is approximately orthogonal to the second β sheet. In such embodiments, the first β sheet has a βα βα βα topology and the first β sheet is flanked on its exterior face by two antiparallel helices.

In some embodiments in accordance with the first aspect of the present invention, the parent protein is a chaperonin or a domain derived from a chaperonin. In some embodiments, the parent protein is the substrate-binding domain of a Group II chaperonin. In yet other embodiments, the parent protein is the substrate-binding domain of the α subunit of the Thermoplasma acidophilum thermosome (residues 214 through 365 of SEQ ID NO: 1). See Waldmann et al., 1995, J. Biol. Chem. Hoppe-Seyler 376 (2), pp. 119-126.

In some embodiments in accordance with the first aspect of the invention, the engineered protein is free of disulfide bonds. In still other embodiments, the randomization of a portion of the primary sequence of the parent protein, to yield the engineered protein, results in a change in the overall number of residues present in the primary sequence of the engineered protein relative to the parent protein. In additional embodiments, the engineered protein domain exhibits an EC₅₀ for a compound that is greater than 1×10³ M⁻¹ and the corresponding parent protein exhibits an EC₅₀ for the compound that is less than 1×10³ M⁻¹. In still other embodiments, when the engineered protein is attached to a surface using N-terminal or C-terminal chemistry, the engineered protein retains the ability to bind to a compound of interest. In some embodiments, the engineered protein includes an N-terminal serine or threonine residue that is used to attach the protein to a surface by selective oxidation of the N-terminal serine or threonine to form a glyoxylyl group or a keto group that is then reacted with a functionality on the surface. The surface functionality may be, for example, an amino-oxy or hydrazine functionality or a heterobifunctional compound bearing both an amino-oxy or hydrazine functionality and a second reactive group that attaches to the surface.

Still other embodiments in accordance with the first aspect of the invention provide a nucleic acid encoding the engineered protein. The nucleic acid is DNA in one embodiment. In another embodiment, the nucleic acid comprises a nucleotide sequence that hybridizes under conditions of high, moderate, or low stringency to nucleotides 760 through 1215 of SEQ ID NO: 2 or a nucleotide sequence that hybridizes under conditions of high, moderate, or low stringency to a polynucleotide that is complementary to nucleotides 760 through 1215 of SEQ ID NO: 2. Additional embodiments provide a nucleic acid in which the overall sequence similarity of the nucleotide sequence of the nucleic acid to nucleotides 760 through 1215 of SEQ ID NO: 2 is characterized by an expectation value that is selected from a range of 1e-4 to 1e-9. Yet other embodiments in accordance with the first aspect of the invention provides a nucleic acid in which the overall sequence similarity of the nucleic acid to nucleotides 760 through 1215 of SEQ ID NO: 2 is characterized by an expectation value that is selected from a range of 1e-4 to 1e-6.

### 4.2 Arrays of Engineered Three-Layer Swiveling Beta/Beta/Alpha Proteins

A second aspect of the present invention provides an array of engineered proteins immobilized on a solid support. In one embodiment, each of the engineered proteins in the array includes an engineered chaperonin domain. In one example, the engineering scheme used to produce this engineered chaperonin domain comprises randomizing select portions of the chaperonin domain of the corresponding parent domain. Another embodiment provides an array comprising a plurality of engineered proteins immobilized on a solid support. Each engineered protein in the array of engineered proteins is derived from the same parent protein and largely retains the scaffold region of that parent protein. The parent protein comprises a three-layer swiveling β/β/α domain. The central beta sheet of the three-layer swiveling β/β/α domain is parallel and the other beta sheet in the three-layer swiveling β/β/α domain is antiparallel. At least one portion of the primary sequence of each engineered protein in the plurality of engineered proteins is determined by an operation of an engineering scheme on the primary sequence of the corresponding parent protein. However, the total length of the at least one portion of the engineered protein that is subjected to the engineering scheme is constrained so that it does not exceed fifty percent of the length of the primary sequence of the engineered protein and so that it comprises at least five percent of the length of the primary sequence of the engineered protein.

In one embodiment in accordance with the second aspect of the invention, the solid support is a bead, slide, or chip. In another embodiment in accordance with the second aspect of the invention, at least one engineered protein in the array of engineered proteins is characterized by an ability to bind to a compound that the corresponding parent protein that includes a chaperonin domain does not specifically bind. In still another embodiment, each engineered protein in the array of engineered proteins is an engineered form of the substrate-binding domain of a Group II chaperonin. In yet another embodiment, each engineered protein in the array of engineered proteins is an engineering product of the substrate-binding domain of the α subunit from the Thermoplasma acidophilum thermosome.

Another embodiment in accordance with the second aspect of the present invention provides an array of engineered proteins in which each engineered protein is derived from a chaperonin domain comprising approximately residues Ser 214 through Asn 365 of the α subunit of the chaperonin from Thermoplasma acidophilum (residues 214 through 365 of SEQ ID NO: 1). In this embodiment, at least one portion of the primary sequence of each engineered protein is subjected to an engineering scheme. The at least one portion includes any combination of a segment that comprises residue 219 (Asp 219) though residue 226 (Lys 226) of SEQ ID NO: 1; a segment that comprises residue 291 (Gln 291) through residue 296 (Asp 296) of SEQ ID NO: 1; a segment that comprises residue 311 (Arg 311) through residue 315 (Lys 315) of SEQ ID NO: 1; anda segment that comprises residue 351 (Lys 351) through 357 (Met 357) of SEQ ID NO: 1.

### 4.3 Methods for Obtaining Engineered Chaperonin Proteins

A third aspect of the present invention provides methods for obtaining an engineered protein that binds to a compound to form a complex. In such methods, the compound is contacted with an array of candidate engineered proteins immobilized on a solid support. Each candidate engineered protein in the array of candidate engineered proteins comprises an engineered chaperonin domain. In one embodiment, the engineering scheme used to produce each engineered chaperonin domain comprises randomizing a portion of the corresponding parent chaperonin domain. The next step in the method comprising obtaining the engineered protein that binds to the compound the protein/compound complex. In some embodiments, the method further comprises further engineering the protein that binds to the compound and forming an array on a solid support with the further engineered proteins.

### 4.4 Methods for Detecting a Compound in a Sample Using Engineered Chaperonins

A fourth aspect of the present invention provides a method for detecting a compound in a sample. In the method, a sample with a candidate protein that binds to a compound is contacted with the compound in order to form a complex. The candidate protein comprises a chaperonin domain in which at least one portion of the primary sequence of the chaperonin domain is engineered. In one embodiment, the engineering scheme used is a randomization scheme. Then, the complex is detected, thereby detecting the compound in the sample. In some embodiments in accordance with the fourth aspect of the invention, the sample is a biological sample.

In some embodiments in accordance with the fourth aspect of the present invention, the candidate protein is immobilized on a bead, chip, or slide. In other embodiments in accordance with the fourth aspect of the invention, the candidate protein is immobilized on a solid support as part of an array of proteins. In such embodiments, each protein in the array of proteins comprises a chaperonin domain having at least one randomized portion. In some embodiments, the complex or the compound is detected by radiography, spectroscopy, fluorescence detection, mass spectrometry, or surface plasmon resonance. In some embodiments of the present invention, the dissociation constant of the complex is less than 10⁻⁶ moles/liter.

### 4.5 Methods for Engineering Chaperonin Mutant

A fifth aspect of the present invention provides an engineered polypeptide that is made by deletion, insertion, replacement or randomization of at least two amino acids from the corresponding portion of a parent chaperonin. However, the sequence of the engineered polypeptide has at least fifty percent total amino acid sequence identity to the corresponding portion of the parent sequence. In some embodiments in accordance with the fifth aspect of the present invention, the engineered chaperonin polypeptide is capable of binding to a compound to form a polypeptide:compound complex having a dissociation constant of less than 10⁻⁶ moles/liter.

### 4.6 Methods for Preparing an Engineered Library of Chaperonin Mutants

A sixth aspect of the present invention provides a method of preparing an engineered library from a set of paired oligonucleotides. The first oligonucleotide in each pair of oligonucleotides includes a region that is complementary to the corresponding second oligonucleotide in each pair of oligonucleotides. At least one oligonucleotide in the set of paired oligonucleotides includes a randomized sequence. The method comprises mixing together, in a different reaction, each pair of paired oligonucleotides in the set of oligonucleotides and performing mutually primed extension using a DNA polymerase and multiple cycles of annealing, extension and denaturation. The reaction products are then mixed together and allowed to perform cycles of mutually primed DNA synthesis. The amplified product is then amplified by PCR using primers specific for the ends of the designed product and cloned into an expression vector.

### 4.7 Additional Methods for Preparing an Engineered Library of Chaperonin Mutants

A seventh aspect of the invention provides a library of engineered proteins. In one embodiment, each engineered protein in the library of engineered proteins comprises a portion of a Group II chaperonin domain that has been subjected to an engineering scheme. In one example, this engineering scheme comprises randomizing at least one portion of the primary sequence of the parent Group TI chaperonin domain. In one embodiment in accordance with the seventh aspect of the invention, each engineered protein in the library of engineered proteins is an engineering product of the substrate-binding domain of the α subunit of the Thermoplasma acidophilum thermosome (residues 214 through 365 of SEQ ID NO: 1). In some embodiments, each of the engineered proteins in the library is attached to a genetically replicable package and the engineered protein that can bind to a compound is identified by performing a binding selection protocol on the engineered proteins in the library. In some embodiments, the binding selection protocol is in accordance with the protocols found in U.S. Pat. No. 5,837,500 to Ladner et al.

In one embodiment, the genetically replicable package is a microbe, a bacterium, a phage, a translationally stalled ribosome, or a protein physically linked to its encoding mRNA or cDNA by a covalent or a non-covalent bond, and the selection protocol used to identify the engineered protein in the library of engineered proteins that binds to the compound is a microbial display, a bacterial display, a phage display, a ribosome display, an mRNA display, or a peptide-on-plasmid display. In one embodiment, the genetically replicable package is a phage, and the method used to identify engineered proteins in the library that bind to the compound is phage display. Suitable bacteriophage include T7, SPbc2, SPP1, phiX174, IEM, T4, UrLamda, P22, M13, f1, fP1, MS2, SPO1, B3, HK97, fXo, λ, and λZAP. In one preferred embodiment, the phage is T7 phage and the engineered proteins in the library are attached to the C-terminus of the major coat protein of this phage.

Another embodiment in accordance with the seventh aspect of the invention provides a library of proteins that comprises a plurality of engineered proteins. The parent protein that corresponds to each engineered protein in the plurality of engineered proteins comprises a three-layer swiveling β/β/α domain. The central beta sheet of the three-layer swiveling β/β/α domain is parallel and the other beta sheet in the three-layer swiveling β/β/α domain is antiparallel. At least one portion of the primary sequence of each engineered protein in the plurality of engineered proteins is determined by an operation of an engineering scheme on the primary sequence of the parent protein. However, the amount of the primary sequence determined by the engineering scheme is subject to constraints. The at least one portion of the primary sequence of the engineered protein that is determined by the operation of the engineering scheme on the primary sequence of the parent protein does not exceed fifty percent of the length of the primary sequence of the engineered protein. Furthermore, the at least one portion of the primary sequence of the engineered protein that is determined by the operation of the engineering scheme on the primary sequence of the parent protein comprises at least five percent of the length of the primary sequence of the engineered protein.

### 4.8 Methods for Determining Whether an Engineered Chaperonin Specifically Binds to a Compound

An eighth aspect of the invention provides a method of determining whether an engineered protein specifically binds to a compound. In this aspect of the invention, the parent protein that corresponds to the engineered protein comprises a three-layer swiveling β/β/α domain. The central beta sheet of the three-layer swiveling β/β/α domain is parallel and the other beta sheet in the three-layer swiveling β/β/α domain is antiparallel. Further, at least one portion of the primary sequence of the engineered protein is determined by an operation of an engineering scheme on the primary sequence of the parent protein. The operation of the engineering scheme on the primary sequence is limited in the sense that the at least one portion of the primary sequence of the engineered protein that is determined by the operation of the engineering scheme on the primary sequence of the parent protein does not exceed fifty percent of the length of the primary sequence of the engineered protein. Further, the operation of the engineering scheme on the primary sequence is limited in the sense that the at least one portion of the primary sequence of the engineered protein that is determined by the operation of the engineering scheme on the primary sequence of the parent protein comprises at least five percent of the length of the primary sequence of the engineered protein. The method in accordance with this eight aspect of the invention comprises contacting the engineered protein with the compound.

### 4.9 Engineered Zinc-Bound or Iron-Bound Proteins

A ninth aspect of the invention provides an engineered protein. In this aspect of the invention, the parent protein that corresponds to the engineered protein has a zinc-bound fold or an iron-bound fold. Furthermore, the primary sequence of the parent protein in this aspect of the invention includes two CXₙC motifs, where X is a residue of any naturally occurring amino acid and n is 1, 2, 3, or 4. At least one portion of the primary sequence of the engineered protein is determined by an operation of an engineering scheme on the primary sequence of the parent protein, with the provisos that: (i) the at least one portion of the primary sequence of the engineered protein that is determined by the operation of the engineering scheme on the primary sequence of the parent protein is at least five percent but does not exceed fifty percent of the length of the primary sequence of the engineered protein.

In some embodiments, engineered protein is attached to a surface such as a chip, slide or bead. In some embodiments, the operation of the engineering scheme comprises wholly or partly randomizing at least one portion of the primary sequence of the parent protein in order to form the engineered protein. In some embodiments, the operation of the engineering scheme comprises altering at least one portion of the primary sequence of the parent protein using a rational scheme in order to form the engineered protein.

In some embodiments, engineered protein has the ability to specifically bind to a compound that the corresponding parent protein does not specifically bind. Such a compound can be, but is not limited to, a hormone, a low molecular weight compound, a peptide, a protein, or an oligonucleotide. In some embodiments, the engineered protein is attached to a surface using N-terminal or C-terminal chemistry but still retains the ability to bind to the compound. In some embodiments, the engineered protein exhibits an EC₅₀ for the compound that is greater than 1×10³ (M⁻¹) while the parent protein exhibits an EC₅₀ for the compound that is less than 1×10³ (M⁻¹).

In some embodiments in accordance with this ninth aspect of the invention, the parent protein is in the rubredoxin-superfamily. In some embodiments, the parent protein is in the rubredoxin family, the desulforedoxin family, or the cytochrome c oxidase subunit F family. In still other embodiments, the engineered protein comprises rubredoxin. In some embodiments, an N-terminal portion of the primary sequence of the parent protein includes an alanine at a position n, a tryptophan at a position n+2, a glutamic acid at a position n+13, and a phenylalanine at a position n+28. In some embodiments, the parent protein has an overall shape that is ellipsoidal and comprises a three-stranded antiparallel β-sheet with a hydrophobic core comprising a plurality of aromatic residues. In some embodiments, the parent protein comprises rubredoxin from Pyrococcus furiousus, Desulfovibrio gigas, Pseudomonas oleovorans, or Clostridium pasteurianum. In one particular embodiment, the parent protein comprises Pyrococcus furious rubredoxin (SEQ ID NO: 31) and the at least one portion of the primary sequence includes any combination of (i) a segment comprising isoleucine 11 of SEQ ID NO: 31; (ii) a segment comprising glycine 17 through glycine 22 of SEQ ID NO: 31; (iii) a segment comprising proline 33 through aspartic acid 35 of SEQ ID NO: 31; (iv) a segment comprising valine 37 of SEQ ID NO: 31; and (v) a segment comprising glycine 42 through serine 46 of SEQ ID NO: 31.

Other embodiments of the present invention provide a nucleic acid encoding an engineered protein in accordance with this ninth aspect of the invention. In some embodiments, the nucleic acid is DNA. In some embodiments, the nucleotide sequence of the nucleic acid hybridizes under conditions of high stringency to SEQ ID NO: 34 or the complement of SEQ ID NO: 34 (FIG. 24). In some embodiments, the nucleotide sequence of the nucleic acid hybridizes under conditions of moderate stringency to SEQ ID NO: 34 or a nucleotide sequence that hybridizes under conditions of moderate stringency to the complement of SEQ ID NO: 34. In some embodiments, the nucleotide sequence of the nucleic acid is at least 50%, at least 65%, at least 80%, or at least 90% identical to SEQ ID NO: 34 or its complement. Other embodiments of the present invention are directed to expression vectors comprising such nucleic acids or host cells comprising such nucleic acids.

### 4.10 Arrays of Engineered Zinc-Bound or Iron-Bound Proteins

A tenth aspect of the present invention provides an array comprising a plurality of engineered proteins immobilized on a solid support. In this aspect of the invention, each engineered protein in the array of engineered proteins corresponds to a parent protein that has a zinc-bound fold or an iron-bound fold. The primary sequence of the parent protein includes two CXₙC motifs, wherein X is a residue of any naturally occurring amino acid and n is 1, 2, 3, or 4. At least one portion of the primary sequence of each of the engineered protein in the plurality of engineered proteins is determined by an operation of an engineering scheme on the primary sequence of the corresponding parent protein. The at least one portion of the primary sequence of each of the engineered proteins in the plurality of engineered proteins is greater than but does not exceed fifty percent of the length of the primary sequence of the engineered protein. In some embodiments, the parent protein comprises rubredoxin from Pyrococcus furiousus, Desulfovibrio gigas, Pseudomonas oleovorans, or Clostridium pasteurianum.

In some embodiments in accordance with this tenth aspect of the invention, the at least one engineered protein in the array of engineered proteins is characterized by an ability to bind to a compound that the parent protein does not bind. By way of example an not limitation, this compound could be a protein, a hormone, a low molecular weight compound, a peptide, or an oligonucleotide.

In some embodiments in accordance with this tenth aspect of the invention, the parent protein comprises Pyrococcus furious rubredoxin (SEQ ID NO: 31) and the at least one portion of the primary sequence includes any combination of (i) a segment comprising isoleucine 11 of SEQ ID NO: 31; (ii) a segment comprising glycine 17 through glycine 22 of SEQ ID NO: 31; (iii) a segment comprising proline 33 through aspartic acid 35 of SEQ ID NO: 31; (iv) a segment comprising valine 37 of SEQ ID NO: 31; and (v) a segment comprising glycine 42 through serine 46 of SEQ ID NO: 31.

### 4.11 Methods for Determining Whether Engineered Zinc-Bound or Iron-Bound Proteins Bind to a Compound

An eleventh aspect of the present invention provides a method of determining whether an engineered protein binds to a compound. The parent protein that corresponds to the engineered protein has a zinc-bound fold or an iron-bound fold. The primary sequence of the parent protein includes two CXₙC motifs, where X is a residue of any naturally occurring amino acid and n is 1, 2, 3, or 4. At least one portion of the primary sequence of the engineered protein is determined by an operation of an engineering scheme on the primary sequence of the parent protein such that the at least one portion is at least five percent but does not exceed fifty percent of the length of the primary sequence of the engineered protein. In some embodiments, the engineered protein is attached to a solid support such as a bead, a slide or a chip. In some embodiments, the engineered protein forms a complex with the compound and the EC₅₀ of the complex is less than 10⁻⁶ moles/liter.

An eleventh aspect of the invention provides a method for using an engineered protein. The method includes the step (a) of contacting a compound with an array of candidate engineered proteins immobilized on a solid support. The array of engineered proteins immobilized on the solid support include the engineered protein. Furthermore, each engineered protein in the array of engineered proteins comprises an engineered rubredoxin. At least one portion of the primary sequence of the engineered rubredoxin is determined by an engineering scheme, with the limitation that the at least one portion of the primary sequence of the engineered rubredoxin is greater than five percent but less than fifty percent of the primary sequence of the engineered rubredoxin. The method further comprises a step (b) of determining whether the engineered protein binds to the compound.

In some embodiments in accordance with the eleventh aspect of the invention includes the step (c) of further engineering the engineered protein that binds to the compound in step (b); the step (d) of forming an array on a solid support with the further engineered proteins of step (c); and the step (e) of repeating step (a) and step (b) using, in step (a), the array of further engineered proteins as the array of candidate engineered proteins.

### 4.12 Methods for Determining Whether a Compound is in a Sample Using Engineered Zinc-Bound or Iron-Bound Proteins

A twelfth aspect of the invention provides a method for detecting a compound in a sample. The method comprises contacting the sample with an engineered protein that specifically binds to the compound. The parent protein that corresponds to the engineered protein has a zinc-bound fold or an iron-bound fold. The primary sequence of the parent protein includes two CXₙC motifs, where X is a residue of any naturally occurring amino acid and n is 1, 2, 3, or 4. Furthermore, at least one portion of the primary sequence of the engineered protein is determined by an operation of an engineering scheme on the primary sequence of the parent protein, with the limitation that the at least one portion of the primary sequence is greater than but does not exceed fifty percent of the length of the primary sequence of the engineered protein. In some embodiments, the method further comprises detecting a complex between the engineered protein and the compound.

In some embodiments, the parent domain comprises rubredoxin. In some embodiments, the engineered protein is immobilized on a bead, a slide or a chip. In some embodiments, the engineered protein is immobilized on the solid support as part of an array of engineered proteins. In some embodiments, the compound is a protein. In some embodiments, the parent protein comprises Pyrococcus furious rubredoxin (SEQ ID NO: 31) (FIG. 21) and the at least one portion of the primary sequence includes any combination of (i) a segment comprising isoleucine 11 of SEQ ID NO: 31; (ii) a segment comprising glycine 17 through glycine 22 of SEQ ID NO: 31; (iii) a segment comprising proline 33 through aspartic acid 35 of SEQ ID NO: 31; (iv) a segment comprising valine 37 of SEQ ID NO: 31; and (v) a segment comprising glycine 42 through serine 46 of SEQ ID NO: 31.

### 4.13 Mutated Rubredoxins

A thirteenth aspect of the present invention provides a mutated rubredoxin protein in which one or more portions of the mutated rubredoxin protein vary by engineering of at least ten amino acids from the corresponding portion of the wild-type rubredoxin sequence. In this aspect of the invention, the primary sequence of the mutated rubredoxin protein has at least 50% total amino acid sequence identity to the wild-type rubredoxin sequence. In some embodiments, the mutated rubredoxin protein is capable of binding to a compound to form a complex, comprising the mutated rubredoxin protein and the compound, that has an EC₅₀ that is less than 10⁻⁶ moles/liter.

### 4.14 Method for Preparing Engineered Rubredoxins

A fourteenth aspect of the invention provides a method of preparing an engineered rubredoxin library from a set of paired oligonucleotides. The first oligonucleotide in each pair of oligonucleotides includes a region that is complementary to the corresponding second oligonucleotide in each pair of oligonucleotides. At least one oligonucleotide in the set of paired oligonucleotides includes a randomized sequence. The method includes a step (a) of mixing together, in a different reaction, each pair of paired oligonucleotides in the set of oligonucleotides and performing mutually primed DNA synthesis using a DNA polymerase; a step (b) of mixing the reaction products of step (a) and performing multiple cycles of denaturation, annealing, and DNA synthesis using a DNA polymarase; a step (c) of amplifying the DNA constructs from step (b) encoding full-length rubredoxin domain library members; and a step (d) of cloning the product of step (c) into an expression vector.

### 4.15 Libraries of Zinc-Bound or Iron-Bound Engineered Proteins

A fifteenth aspect of the invention provides a library of proteins that comprises a plurality of engineered proteins. The parent protein that corresponds to each engineered protein in the library has a zinc-bound fold or an iron-bound fold and the primary sequence of the parent protein includes two CXₙC motifs, where X is a residue of any naturally occurring amino acid and n is 1, 2, 3, or 4. At least one portion of the primary sequence of each engineered protein in the plurality of engineered proteins is determined by an operation of an engineering scheme on the primary sequence of the parent protein, with the limitation that the at least one portion of the primary sequence of the engineered protein is at least five percent but does not exceed fifty percent of the length of the primary sequence of the engineered protein.

In some embodiments in accordance with this fifteenth aspect of the invention, the parent protein is in the rubredoxin-superfamily. In some embodiments, the parent protein is in the rubredoxin family, the desulforedoxin family, or the cytochrome c oxidase subunit F family. In some embodiments, the parent protein comprises Pyrococcus furious rubredoxin (SEQ ID NO: 31) and each of the at least one portion of the primary sequence of each engineered protein in the library of engineered proteins is selected from the group consisting of (i) a segment comprising isoleucine 11 of SEQ ID NO: 31; (ii) a segment comprising glycine 17 through glycine 22 of SEQ ID NO: 31; (iii) a segment comprising proline 33 through aspartic acid 35 of SEQ ID NO: 31; (iv) a segment comprising valine 37 of SEQ ID NO: 31; and (v) a segment comprising glycine 42 through serine 46 of SEQ ID NO: 31.

In some embodiments, each of the engineered proteins in the plurality of engineered proteins is attached to a genetically replicable package. In some embodiments, the genetically replicable package is a bacteriophage. In some embodiments, the bacteriophage is T7, SPbc2, SPP1, phiX174, IEM, T4, UrLamda, P22, M13, f1, P1, MS2, SPO1, B3, HK97, fXo, or λ.

### 4.16 Methods of Making Engineered Zinc-Bound or Iron-Bound Engineered Proteins

A sixteenth aspect of the invention provides a method of making an engineered protein. The method comprises subjecting at least one portion of the primary sequence of a parent protein to an engineering scheme in order to produce the engineered protein, with the limitation that the parent protein has a zinc-bound fold or an iron-bound fold and the primary sequence of the parent protein includes two CXₙC motifs, where X is a residue of any naturally occurring amino acid and n is 1, 2, 3, or 4. Furthermore, the at least one portion of the primary sequence of the engineered protein is greater than but does not exceed fifty percent of the length of the primary sequence of the engineered protein.

In some embodiments in accordance with this sixteenth aspect of the invention, the engineering scheme is a pseudo-randomization scheme and the step of subjecting the at least one portion of the primary sequence of the parent protein to an engineering scheme results in the randomization of the at least one portion of the primary sequence. I some embodiments, the engineering scheme is a randomization scheme and the step of subjecting the at least one portion of the primary sequence of the parent protein to an engineering scheme results in the pseudo-randomization of the at least one portion of the primary sequence.

### 5. BRIEF DESCRIPTION OF THE DRAWINGS

Additional objects and features of the invention will be more readily apparent from the following detailed description and appended claims when taken in conjunction with the drawings, in which:

FIG. 1 is the β-strand and loop topology (A) and MOLSCRIPT representation (B) (Kraulis, J. Appl. Cryst. 24, 946-950, 1991) of the 10^{th} type III domain of human fibronectin.

FIG. 2 is a flow chart illustrating process steps used to identify a protein that may function as a scaffold in accordance with an embodiment of the present invention.

FIG. 3A shows the protein sequence of the α subunit of Thermoplasma acidophilum thermosome chaperonin (SWISSPROT accession number P48424; SEQ ID NO: 1). The fragment found in the crystal structure of the α subunit of Thermoplasma acidophilum thermosome chaperonin (Brookhaven PDB identifier 1ASX) is in bold text.

FIG. 3B shows the nucleic acid sequence of the α subunit of the Thermoplasma acidophilum thermosome (NCBI accession number Z46649; SEQ ID NO: 2), with bold text representing the sequence of the fragment of this subunit that encodes the protein used to solve the crystal structure.

FIG. 4 illustrates a ribbon diagram of the substrate-binding domain of the α subunit of Thermoplasma acidophilum thermosome (residues 214 through 365 of SEQ ID NO: 1) that was determined by x-ray crystallography (Brookhaven PDB identifier 1ASX) in which the locations of randomized loops in accordance with one embodiment of the invention are illustrated.

FIG. 5 illustrates the nucleic acid sequence of a randomized library based on the substrate-binding domain of the α subunit of the Thermoplasma acidophilum thermosome, where randomized nucleotides are represented by a “1”, “2”, or “3” (SEQ ID NO: 3).

FIG. 6 shows the primers used to create randomized loops in the substrate-binding domain of the α subunit of Thermoplasma acidophilum thermosome in accordance with one embodiment of the present invention.

FIG. 7 illustrates the progress of a biopanning selection that was used to identify phage that express an engineered protein that binds to mouse monoclonal antibodies.

FIG. 8 illustrates a binding curve for the engineered protein clone LO42 in an ELISA assay in which immobilized mouse monoclonal antibody HP6054 was exposed to serial dilutions of the engineered protein LO42.

FIG. 9 illustrates the progress of a biopanning selection that was used to identify phage that express an engineered protein that binds to human chorionic gonadotropin.

FIG. 10 illustrates a binding curve for the engineered protein clone SP4-5 in an ELISA assay in which immobilized human chorionic gonadotropin was exposed to serial dilutions of the engineered protein SP4-5.

FIG. 11 illustrates the progress of a biopanning selection that was used to identify phage that expresses an engineered protein that binds to human leptin.

FIG. 12 illustrates a binding curve for the engineered protein clone 285-89-8 in an ELISA assay in which immobilized leptin was exposed to serial dilutions of the engineered protein 285-89-8.

FIG. 13A shows the top view of engineered protein arrays in accordance with an embodiment of the present invention.

FIG. 13B shows a cross-sectional view of an individual patch of the array of FIG. 13B in accordance with an embodiment of the present invention.

FIG. 13C shows a cross-sectional view of a row of monolayer-covered patches of FIG. 13A in accordance with an embodiment of the present invention.

FIG. 14 shows the immobilization of an engineered protein on a monolayer-coated substrate via an affinity tag in accordance with an embodiment of the present invention.

FIG. 15A and FIG. 15B show a cross-sectional view of chips that include pillars.

FIGS. 16 and 17 show a cross-sectional view of pillars with affinity structures.

FIG. 18 shows a perspective view of a dispenser.

FIG. 19 shows a perspective view of a chip embodiment.

FIG. 20 shows a perspective view of an assembly embodiment.

FIG. 21 illustrates the protein sequence of rubredoxin (SEQ ID NO: 31) that was determined by x-ray crystallography (Brookhaven PDB identifier 1ASX) in which the locations of randomized loops in accordance with one embodiment of the invention are illustrated.

FIG. 22 illustrates a ribbon diagram of rubredoxin (Brookhaven PDB identifier 1BRF) in which the location of randomized loops in accordance with one embodiment of the present invention is illustrated.

FIG. 23 illustrates rubredoxin from Pyrococcus furiosus with gaps introduced (SEQ ID NO: 32), and a library of rubredoxin mutants (SEQ ID NO: 33) that were made in accordance with one embodiment of the present invention. In FIG. 23, periods represent gaps.

FIG. 24 illustrates the nucleotide sequence of rubredoxin from Pyrococcus furiosus (SEQ ID NO: 34).

FIG. 25 illustrates binding curves for engineered rubredoxin mutants in accordance with one embodiment of the present invention.

FIG. 26 illustrates an engineered rubredoxin library in accordance with one embodiment of the present invention.

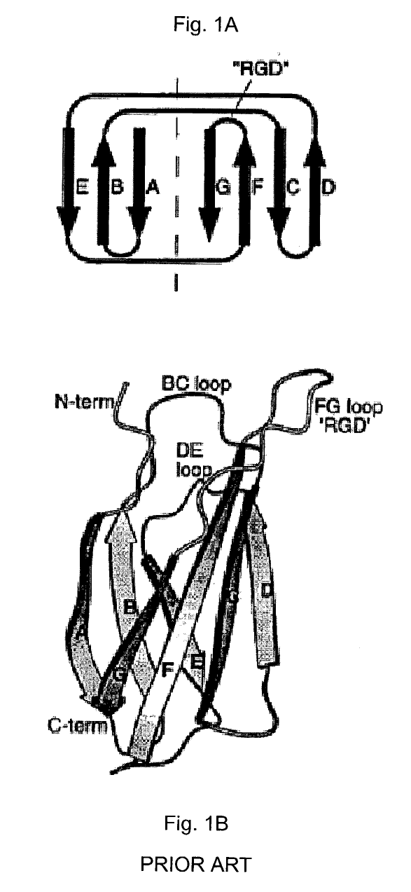

### 6. DESCRIPTION OF THE PREFERRED EMBODIMENTS

The present invention provides a library of engineered proteins that are produced by subjecting a parent protein to an engineering scheme. The engineering scheme changes amino acid residues that are not critical to conferring or maintaining the basic three-dimensional structure (fold) of the parent protein, such as those residues in solvent-exposed turns and loops. The engineering scheme does not alter the amino acid residues that make up the structural “scaffold” of the parent protein, e.g., residues that confer and maintain the basic three-dimensional fold of the protein. The term “parent protein” refers to any protein that is subjected to an engineering scheme in order to form a library of engineered proteins. Each engineered protein in the library presents one or more engineered sequences while retaining the overall protein fold adopted by the parent protein. In one embodiment, the engineering scheme used to produce the engineered proteins of the present invention comprises randomizing one or more portions of the primary sequence of the parent scaffold. Preservation of the parent protein fold in the library of engineered proteins improves the solubility and stability of the library proteins, and constrains the conformations of the engineered sequences and the structural relationships between them in cases where more than one engineered sequence exists within a given engineered protein.

The engineering schemes used in the present invention include randomization schemes as well as pseudo-randomization schemes. In the randomization schemes, the one or more portions of the primary sequence of the engineered protein are randomized. Typically, this randomization does not result in an increase or decrease in the absolute length of the portions of the primary sequence that is randomized. That is, each portion in the engineered protein that is randomized has the same length as the respective portion in the parent protein. However, in some situations, it is desirable to increase or decrease the length of the primary sequence upon randomization. For example, if a portion of the primary sequence to be randomized codes for a solvent accessible loop in the parent protein, it may be desirable to insert extra residues into the loop or to remove residues from the loop. In such instances, the length of the portion of the primary sequence that is subjected to randomization will respectively increase or decrease as a result of the randomization scheme. The pseudo-randomization schemes encompassed in the present invention are similar to the randomization schemes, with the exception that certain positions are held constant within the portions of the primary sequence that are subjected to randomization. Thus, for example, consider the case where a portion of the primary sequence is determined by a pseudo-randomization scheme. In this example, the portion to be pseudo-randomized is twelve bases long. The exemplary pseudo-randomization scheme calls for the second codon within twelve bases to be preserved so that the residue in the protein coded by the second codon remains fixed. Psuedo-randomization schemes are advantageous because they allow for randomization of a region of the parent protein that includes residues that are highly conserved throughout the chaperonin family or the rubredoxin family or that make important contacts that stabilize the protein fold.

In one embodiment of the present invention, engineered proteins are used to select and screen for binding affinity to specific compounds. Furthermore, the engineered proteins of the present invention may be attached to fixed surfaces, such as addressable chips or slides, in order to provide an array of engineered proteins. This array of engineered proteins is used to determine the identity and amounts of proteins in a sample, based on the binding of sample proteins to the engineered proteins, coupled with knowledge of the binding specificity of the engineered proteins for proteins that may be present in samples. In one embodiment of the present invention, engineered proteins are attached to fixed surfaces using either N-terminal or C-terminal chemistries. The engineered proteins of the present invention have been designed so that they are generally stable under reducing conditions. In one embodiment, parent proteins are selected from organisms that are tolerant of high temperatures. Proteins selected from such organisms are very stable. Advantageously, libraries of engineered proteins derived from such parent proteins have highly desirable stability characteristics.

### 6.1 Identification of Scaffolds of the Present Invention

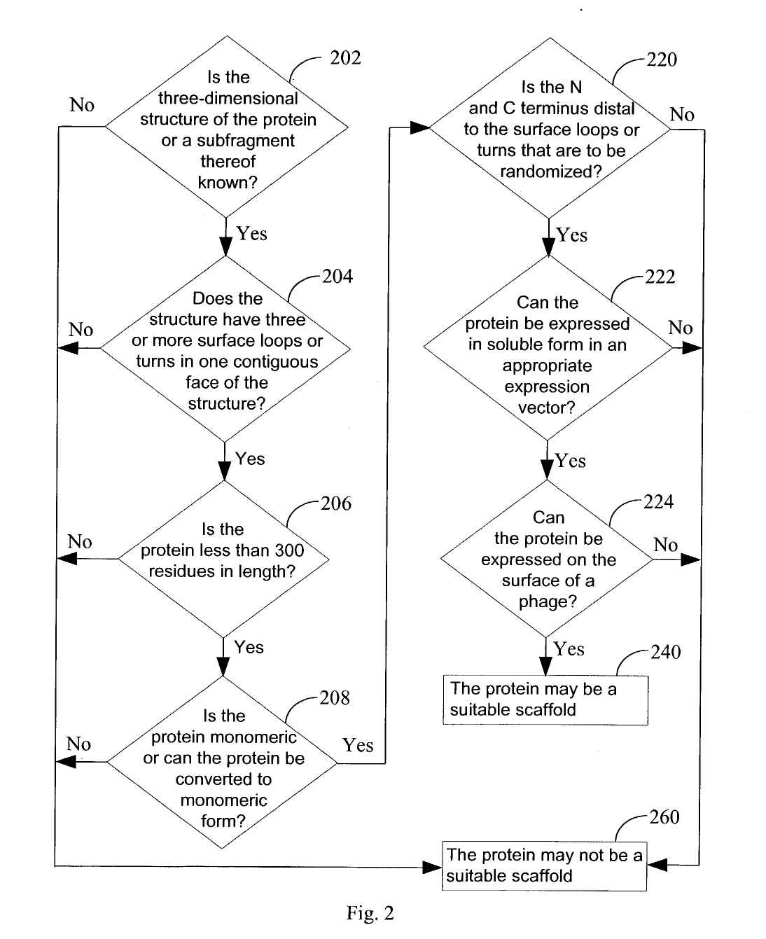

The scaffolds suitable for use in the present invention are first identified using a novel approach that is illustrated in FIG. 2. In this approach, a large number of proteins are considered. Then, the various steps illustrated in FIG. 2 are used to eliminate from consideration many of the reviewed proteins.

Step 202. At this stage, a determination is made as to whether the three-dimensional structure of the protein or a subfragment of the protein is known. If not (202-No), the protein is rejected as a possible parent protein and source of a scaffold. A protein for which the three-dimensional structure is not known is considered disadvantageous because the three-dimensional structure provides a basis for determining which regions of the protein can be randomized without disruption of the overall protein fold, as well as the structural relationships between such regions. Disruption of the overall protein fold often results in decreased protein solubility as well as protein stability, and it is generally found that unstructured polypeptides have poor affinity for compounds. In some embodiments, a protein is not rejected (202-No) if a homology model is available for the protein. Accordingly, if the three-dimensional structure of the protein is known or there was a reliable three-dimensional model available for the protein (202-Yes), the protein is not eliminated from consideration.

Step 204. Proteins with known or modeled three-dimensional structure are examined to determine whether they have three or more surface loops or turns on one contiguous face of the structure. These surface loops or turns constitute diversifiable regions and can be subjected to engineering without compromising the overall structural fold of the parent protein. The requirement for three or more surface loops is imposed based on the assumption that the affinity of engineered proteins for the compounds to which they bind will be a function of the total surface area that interacts with the compounds, and therefore of the total surface area available to the engineering scheme. Randomization of the three or more surface loops or turns in proteins that present these loops or turns on one contiguous face produces a much larger engineered molecular surface than would be generated using proteins that do not have three or more surface loops or turns on one contiguous face. Proteins that do not have three or more surface loops or turns on one contiguous face are rejected (204-No) while proteins that do have three or more surface loops or turns on one contiguous face (204-Yes) are subjected to further scrutiny.

Step 206. One embodiment of the present invention provides engineered protein libraries that can be affixed to addressable chips or slides. In such embodiments, the parent protein used to derive such libraries is highly stable, has excellent protein expression and solubility characteristics, and is reduction-resistant. To select for parent proteins that have some likelihood of possessing one or more of these desired properties, a 400 residue cutoff is imposed in step 206. Proteins having more than 400 residues are rejected (206-No) whereas proteins with less than 400 residues are subjected to further scrutiny (206-Yes). It will be appreciated that the choice of 400 residues is somewhat arbitrary. In alternative embodiments, a cutoff of 200 residues is used. In another embodiment, a cutoff of 300 residues is used. In still another embodiment, a cutoff of 500 residues is used.

Step 208. In step 208, the criterion that the parent protein exists in a monomeric form or can be converted to monomeric form is imposed. This criterion is imposed to improve the chances that proteins passing all criteria imposed will have desired properties, including excellent protein expression, protein solubility, and protein stability. Proteins that are not found in monomeric form or that could not be converted to monomeric form are rejected (208-No) whereas proteins found in monomeric form or that could be converted to monomeric form are subjected to further scrutiny (208-Yes). In one embodiment, proteins that form oligomers are not rejected as long as the monomeric protein can be expressed in soluble form.

Step 220. Several types of recombinant tags can be introduced into either terminus of proteins in which both termini are at regions of the protein that are distal to the engineered loops that confer the ability of the engineered protein to bind to a compound. This is advantageous because affinity tags are occasionally non-functional, depending on their context. That is, some affinity tags only work when they are attached to the N-terminus of a protein whereas other affinity tags are only functional when they are attached to the C-terminus of a protein. Also, tags can sometimes interfere with the function of a protein, either by affecting its folding, its solubility, or some other physical property. The degree to which a tag interferes with the function of a protein may also depend on the placement of the tag (N or C-terminal). For these reasons, it is desirable to select proteins that provide the freedom to attach tags to either terminus of the protein, rather than just a single terminus.

In the case of the fibronectin scaffold discussed previously, only the C-terminus can be used to attach a tag sequence. This is because the C-terminus is on a side of the protein that opposes the engineered face whereas the N-terminus is on the same side of the protein as the engineered face. Thus, the N-terminus is not an appropriate part of the sequence to include an affinity tag. It is advantageous to attach affinity tags to the N-terminus of a protein because there are certain surface-attachment methods that can only be performed at the N-terminus of proteins. One such method relies on generating a protein with an N-terminal serine or threonine residue. The N-terminal hydroxyl group of these residues can be selectively oxidized to form a glyoxylyl group, or a keto group. These unique chemical functionalities can then be reacted with, for example, aminooxy- or hydrazine-functionalized surfaces, or to heterobifunctional compounds bearing both an aminooxy or hydrazine functionality and a second reactive group for surface attachment (Gaertner et al., 1992, Bioconjugate Chemistry 3, pp. 262-268; Geoghegan & Stroh, 1992, Bioconjugate Chemistry 3, pp. 138-146; Gaertner et al., 1994, J Biol. Chem 269, pp. 7224-7230; Alouani et al., 1995, Eur. J. Biochem. 227, pp. 328-334; Gaertner & Offord, 1996, Bioconjugate Chem. 7, pp. 38-44). There is also the possibility to selectively derivatize proteins bearing N-terminal cysteine residues to surfaces (or heterobifunctional compounds, as described above) using the chemistry developed for “native chemical ligation” of peptides (Dawson et al., 1994, Science 266, pp. 776-779).

Proteins in which the N and/or C terminus are not distal to the surface loops or turns that are to be engineered are rejected (220-No) based on the assumption that termini that are proximate to the surface to be engineered may not be available for derivitization. Proteins in which both the N and/or C terminus are distal to regions to be engineered are subjected to further analysis (220-Yes).

Step 222. The next question asked is whether the protein can be expressed in soluble form in an appropriate expression system. Such information is often found in primary references that describe the protein. Alternatively some experimentation may be required in order to determine whether the protein can be expressed in soluble form. Those proteins that cannot be expressed in soluble form are rejected (222-No) while those proteins that can be expressed in soluble form are further studied (222-Yes). A preferred expression system is the bacteria E. coli, which is compatible with various phage display systems.

Step 224. One method to identify a protein in a protein library that has the ability to bind to a compound of interest is to display the protein on a phage and perform a technique called phage display. Therefore, in step 224, a determination is made as to whether the protein can be expressed on the surface of a phage. Methods used to determine whether a protein can be expressed on the surface of a phage are well known in the art and some methods for expressing a protein on the surface of a phage are discussed in the experimental section below. In some embodiments of the present invention, proteins that can be expressed on the surface of a phage (224-Yes) and that pass all other criteria specified in FIG. 2 are considered to be suitable parent protein candidates and are therefore sources of suitable protein scaffold for further engineering (240). Proteins that fail step 224 (step 224-No) or any of the other criteria illustrated in FIG. 2 are considered not suitable for scaffold study (260).

### 6.2 Proteins that Provide Useful Scaffolds

6.2.1 Three-Layer Swiveling β/β/α Domains Generally

Using the novel criteria for selection of a parent protein and associated protein scaffold described in Section 6.1, the potential utility of proteins that include a three-layer swiveling β/β/α domain was discovered. The three-layer swiveling β/β/α domain is described in the Structural Classification of Protein (SCOP) database (See Murzin et al., 1995, J. Mol. Biol. 247, pp. 536-540).

Murzin et al. have classified proteins with known folds based on evolutionary relationships and on the principles that govern their three-dimensional structure. This classification is hierarchical in nature. In the classification system, domains of large proteins that have multiple protein domains are treated individually. A protein domain is a region within a protein that can fold independently of any other regions within the same protein, and has a well-defined tertiary structure (See Cuff et al., 1999, Proteins 34, pp. 508-519; Russell et al., 1996, J. Mol. Biol. 259, pp. 349-365; and Siddiqui & Barton, 1995, Protein Science 5, pp. 872-884). Murzin et al. cluster proteins together into families if (i) they have residue identities of 30% or greater or (ii) they have lower sequence identity but have very similar structure and function.

The swiveling β/β/α domain as classified by Murzin et al. includes a central beta sheet that is flanked on one face by a beta sheet and on the other face by one or more alpha helices. The central beta sheet is parallel, and the other beta sheet is antiparallel. The swiveling β/β/α domain includes, but is not limited to, residues 377-505 of the pyruvate phosphate dikinase from Clostridium symbiosum (Herzberg et al., 1996, Proc. Natl. Acad. Sci. U.S.A. 93, pp. 2652; representative PDB accession number 1DIK); the N-terminal domain of enzyme I of the E. coli PEP:sugar phosphotransferase system (Liao et al., 1996, Structure 4 pp. 861; representative PDB accession number 1ZYM); the C-terminal domain of Aconitase (Lauble et al., 1992, Biochemistry 31 pp. 2735; Lauble et al., 1994, J. Mol. Biol. 237, pp. 437; representative PDB accession numbers 1ACO and 7ACN); the small subunit N-terminal domain of carbamoyl phosphate synthetase (Thoden et al., 1998, Biochemistry 37, pp. 8825; representative PDB accession number 1A9X); the apical domain of the transferrin receptor ectodomain (Bennett et al., 2000, Nature 403, pp. 46; representative PDB-accession number 1DE4); as well as the substrate-binding domain of GroEL or GroEL-like chaperonins (Chen et al., 1999, Cell 99, pp. 757; Walsh et al., 1999, Acta Crystallogr., Sect. D 55 pp.1168; Klumpp et al., 1997, Cell 91, pp. 263; representative PDB accession numbers 1DK7, 1SRV, and 1ASX).

6.2.2 GroEL or GroEL-Like Chaperonins

The substrate-binding domain of GroEL or GroEL-like chaperoning, as classified by SCOP, include the substrate-binding domain of group I and group II chaperoning. Group I of the chaperonin family includes the chaperonins of bacteria, mitochondria, and chloroplasts. The archaeal theromosomes and the eukaryotic cytosolic chaperonin TriC/CCT (Trent et al., 1991, Nature 354, pp. 490-493) constitute group II of the chaperonin family. For a review of the chaperonin family, see Willison and Horwich, in The Chaperonins, R. J. Ellis, ed. (San Diego, Calif., Academic Press). Chaperonins represent a distinct family of proteins that assist in the folding of newly synthesized proteins or the refolding of stress-denatured proteins (Ellis, The Chaperonins, San Diego, Calif.; Academic Press, 1996). Chaperonins include an ATPase domain, an intermediate domain, and a substrate-binding domain.

The substrate-binding domain of GroEL or GroEL-like chaperoning, as classified by SCOP, includes the substrate-binding domain of the T. acidophilum theromosome, which is an Archaeal group chaperonin (Klumpp et al., 1997, Cell 91, pp. 263.). In archaea, the chaperonin family is represented by the thermosomes (Phipps et al., 1993, Nature 361, pp. 475-477).

Often, chaperonins include different subunits. For example, Thermoplasma acidophilum has two thermosome subunits. The two subunits are referred to as the α and β subunits of Thermoplasma acidophilum thermosome. In Thermoplasma acidophilum, the α and β thermosome subunits alternate within multi-membered rings that stack together (Nitsch et al., J. Mol. Biol. 267, 142-149, 1997). The α and β subunits of Thermoplasma acidophilum thermosome share 63% sequence identity. Further, the α and β subunits of Thermoplasma acidophilum thermosome share a high degree of sequence identity to eukaryotic cytosolic chaperonin TriC/CCT (Trent et al., Nature 354, pp. 490-493, 1991).

Although the overall organization of the subunits, the binding of substrate to a central cavity, and the ATP-dependent substrate release is common to group I and group II chaperonins, there is no significant sequence similarity between the substrate-binding domains of group I and group II chaperoning. The structural comparison of a group I chaperonin substrate-binding domain (GroEL; Zahn et al. Proc. Natl. Acad. Sci. USA 93, 15024-15029, 1996) to a group II chaperonin substrate-binding domain (the α subunit of Thermoplasma acidophilum thermosome) reveals that both domains include a swiveling β/β/α domain. The β-sandwich architecture comprises two orthogonal sheets in which a central β sheet has a βα βα βα topology and is flanked on its exterior face by two antiparallel helices. The few residues conserved between the substrate-binding domain of the α subunit of Thermoplasma acidophilum thermosome (group II chaperonin) and GroEL (group I chaperonin) are predominantly found in the hydrophobic core of the β sandwich (Klumpp et al., 1997, Cell 91, pp. 263-270).

One embodiment of the present invention provides a mutated chaperonin polypeptide. One or more portions of the mutated chaperonin polypeptide vary by engineering of at least two amino acids, at least five amino acids, at least ten amino acids, or at least 25 amino acids or more from the corresponding portion of the wild-type substrate-binding domain of a chaperonin. Further the sequence of the mutated chaperonin polypeptide has at least fifty percent total amino acid sequence identity to the wild-type chaperonin sequence.

6.2.3 The Alpha Subunit of Thermoplasma acidophilum Thermosome

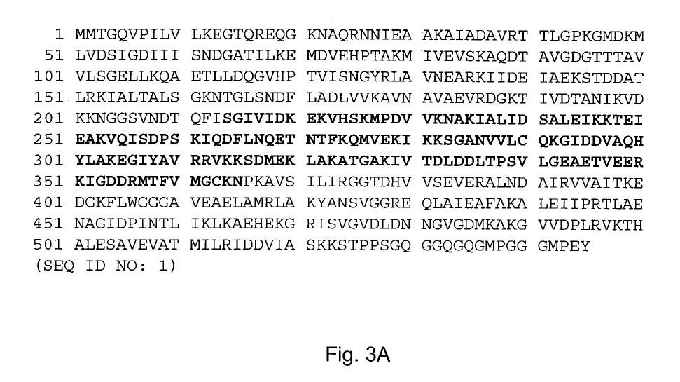

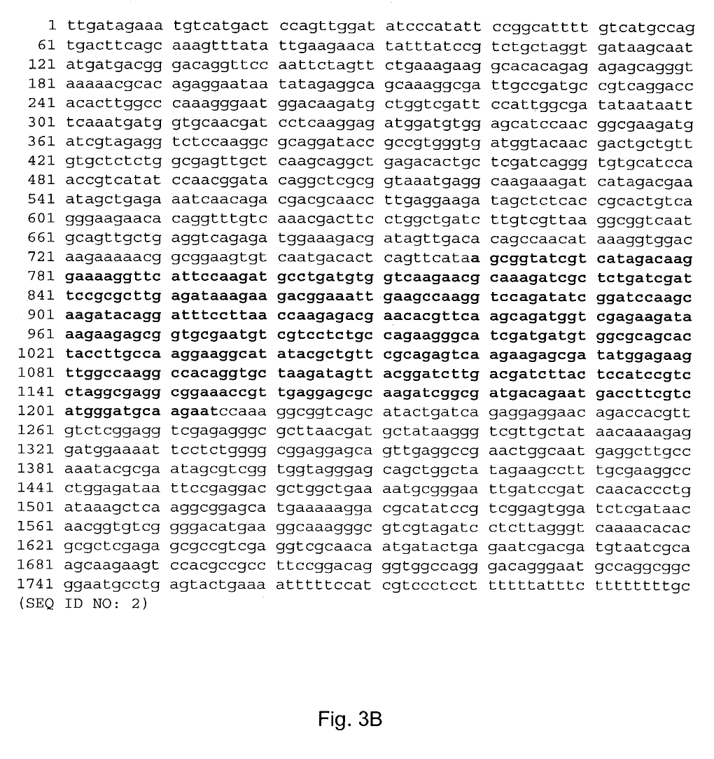

One aspect of the present invention provides engineered proteins that are derived from the substrate-binding domain of the α subunit of a Thermoplasma acidophilum thermosome. The α subunit of the Thermoplasma acidophilum thermosome contains a domain that starts at residue 214 and terminates at residue 365 of SEQ ID NO: 1 (FIG. 3A). In this aspect of the invention, the engineered proteins are formed by randomizing select regions of the α subunit of the Thermoplasma acidophilum thermosome.

Techniques for randomizing portions of the primary sequence of a protein are known in the art. In one technique, a library of engineered proteins is constructed from synthetic DNA oligonucleotides by mutually primed extension of the DNA oligonucleotides. Certain positions in these oligonucleotides have degenerate positions that correspond to the regions of the primary sequence of the parent protein that is randomized to provide the resulting library of engineered proteins.

Generally, residues that are solvent-exposed and that lie on one contiguous face of the parent protein are subjected to an engineering scheme such as randomization. In one embodiment, a residue is considered solvent-exposed if over twenty percent of the surface area of the residue is contacted by a 1.4 Angstrom test sphere as described by Connolly. (See Connolly, 1983, Science 221, pp. 709-713). Similarly, a solvent-accessible atom is one having over twenty percent of its surface area contacted by a 1.4 Angstrom test sphere. With this in mind, one embodiment of the present invention provides libraries of engineered proteins in which each engineered protein in the library includes the substrate-binding domain of the α subunit of Thermoplasma acidophilum thermosome (residue 214 through residue 365 of SEQ ID NO: 1) in which at least one portion of the primary sequence of the thermosome is subjected to an engineering scheme such as randomization. The portions that are engineered in this embodiment include any combination of the following: (i) a segment ranging from residue 219 (Asp 219) to residue 226 (Lys 226) of SEQ ID NO: 1; (ii) a segment ranging from residue 291 (Gln 291) to residue 296 (Asp 296) of SEQ ID NO: 1; (iii) a segment ranging from residue 311 (Arg 311) to residue 315 (Lys 315) of SEQ ID NO: 1; and (iv) a segment ranging from residue 351 (Lys 351) to residue 357 (Met 357) of SEQ ID NO: 1.

It will be appreciated that there is a high degree of sequence similarity between the α and β subunits of Thermoplasma acidophilum thermosome. Therefore, one embodiment of the present invention provides engineered proteins derived from the β subunit of Thermoplasma acidophilum thermosome, in which any combination of the portions of the β subunit that corresponds to Asp 219 to Lys 226 of SEQ ID NO: 1, Gln 291 to Asp 296 of SEQ ID NO: 1, Arg 311 to Lys 315 of SEQ ID NO: 1, and Lys 351 to Met 357 of SEQ ID NO: 1, are subjected to an engineering scheme such as randomization. Further, because the α (SEQ ID NO: 1) and β subunits (SEQ ID NO: 24) of Thermoplasma acidophilum thermosome share a high degree of sequence identity to eukaryotic cytosolic chaperonin TriC/CCT (Trent et al., Nature 354, pp. 490-493, 1991), one embodiment of the present invention provides TriC/CCT in which any combination of the portions of the TriC/CCT that correspond to Asp 219 to Lys 226 of SEQ ID NO: 1, Gln 291 to Asp 296 of SEQ ID NO: 1, Arg 311 to Lys 315 of SEQ ID NO: 1, and Lys 351 to Met 357 of SEQ ID NO: 1, are subjected to an engineering scheme such as randomization. One embodiment that may be used to produce therapeutically efficacious binding proteins provides a human TriC/CCT in which any combination of the portions of the TriC/CCT that correspond to Asp 219 to Lys 226 of SEQ ID NO: 1, Gln 291 to Asp 296 of SEQ ID NO: 1, Arg 311 to Lys 315 of SEQ ID NO: 1, and Lys 351 to Met 357 of SEQ ID NO: 1 are subjected to an engineering scheme such as randomization.

One embodiment of the present invention provides an engineered protein in which the corresponding parent protein comprises the α subunit of Thermoplasma acidophilum thermosome (residue 214 to residue 365 of SEQ ID NO: 1). In this embodiment, the residue in the engineered protein that corresponds to Val 313 of SEQ ID NO: 1 is conserved as a valine. Another embodiment of the present invention provides an engineered protein in which the corresponding parent protein comprises the α subunit of Thermoplasma acidophilum thermosome (residue 214 to residue 365 of SEQ ID NO: 1). In this embodiment, the residues in the engineered protein that correspond to Asp 299 and His300 of SEQ ID NO: 1 are randomized.

6.2.4 Engineered Proteins that Comprise a Three-Layer Swiveling β/β/α Domain

In some embodiments of the present invention, engineered proteins are derived from a parent protein. In some embodiments, the parent protein comprises a three-layer swiveling β/β/α domain. It will be appreciated that the parent protein does not have to be a full-length naturally occurring protein. In fact, in some embodiments, the parent scaffold protein is a fragment or a portion of a naturally occurring protein. Thus, any protein or peptide that includes a three-layer swiveling β/β/α domain is considered a parent protein. A parent protein may include amino acids that are extraneous to the three-layer swiveling β/β/α domain. In fact, a parent protein may include any number of additional domains. In some embodiments, a parent protein has any number of mutations, including deletions, insertions and/or substitutions. In some embodiments, the extent to which the three-layer swiveling β/β/α domain is mutated is subject to the limitation that the parent protein maintains a three-layer swiveling β/β/α fold. In some embodiments, the parent protein has less than 5 mutations, less than 10 mutations or less than 20 mutations. In still other embodiments, the parent protein has less than 5 residues deleted from one or more portions of the three-layer swiveling β/β/α domain, less than 10 residues deleted from one or more portions of the three-layer swiveling β/β/α domain, or less than 25 residues deleted from one or more portions of the three-layer swiveling β/β/α domain. In still other embodiments, the parent protein has less than 5 residues inserted into one or more portions of the three-layer swiveling β/β/α domain, less than 10 residues inserted into one or more portions of the three-layer swiveling β/β/α domain, or less than 25 residues inserted into one or more portions of the three-layer swiveling β/β/α domain.

The central beta sheet of the β/β/α domain of the parent protein is parallel and the other beta sheet is antiparallel. In one embodiment, at least one portion of the primary sequence of each engineered protein is randomized. In some embodiments, the at least one portion of the primary sequence that is determined by the operation of an engineering scheme collectively represents less than five percent of the total sequence of the parent. In more preferred embodiments, the at least one portion of the primary sequence that is determined by the operation of an engineering scheme collectively represents less than ten percent of the total sequence of the parent protein. In still more preferred embodiments, the at least one portion of the primary sequence that is determined by operation of an engineering scheme collectively represents less than fifteen percent, twenty percent, twenty-five percent, or more, of the total sequence of the parent protein. In other embodiments, the at least one portion of the primary sequence that is determined by the operation of an engineering scheme collectively represents less than thirty-five percent or more of the total sequence of the parent protein.

One embodiment of the present invention provides an engineered protein. The parent protein that corresponds to the engineered protein comprises a three-layer swiveling β/β/α domain. The central beta sheet of the three-layer swiveling β/β/α domain is parallel and the other beta sheet in the three-layer swiveling β/β/α domain is antiparallel. Further, at least one portion of the primary sequence of the engineered protein is determined by an operation of an engineering scheme on the primary sequence of the parent protein. Suitable engineering schemes include randomization and pseudo-randomization schemes. In this embodiment, the total length of the at least one portion of the primary sequence of the engineered protein that is determined by an operation of the engineering scheme is subject to constraints. In some cases, the at least one portion of the primary sequence of the engineered protein that is determined by operation of the engineering scheme on the primary sequence of the parent protein does not exceed thirty percent, thirty-five percent, forty percent, fifty percent, fifty-five percent, sixty percent, sixty-five percent, seventy percent, seventy-five percent, or eighty percent of the length of the primary sequence of the engineered protein. Furthermore, the at least one portion of the primary sequence of the engineered protein that is determined by the operation of the engineering scheme on the primary sequence of the parent protein comprises at least three percent, five percent, eight percent, ten percent, fifteen percent, twenty percent, twenty-five percent, thirty percent, thirty-five percent, forty-percent, or forty-five percent of the length of the primary sequence of the engineered protein.

6.2.5 Rubredoxin and Rubredoxin Related Proteins

Using the novel criteria for selection of a parent protein and associated protein scaffold described in Section 6.2, the potential utility of proteins that have a rubredoxin-like fold were discovered. The rubredoxin-like fold is described in the Structural Classification of Protein (SCOP) database (See Murzin et al., 1995, J. Mol. Biol. 247, pp. 536-540). The rubredoxin-like fold is characterized as a zinc-bound fold or an iron-bound fold by a protein having a primary sequence that includes two CXₙC motifs where X is any naturally occurring amino acid residue and n is 1, 2, 3, or 4. Accordingly, one embodiment of the present invention provides an engineered protein. The parent protein that corresponds to this engineered protein comprises a protein having a rubredoxin-like fold. That is the parent protein has a zinc-bound fold or an iron-bound fold and the primary sequence of the parent protein includes two CXₙC motifs, where X is a residue of any naturally occurring amino acid and n is 1, 2, 3, or 4. At least one portion of the primary sequence of the engineered protein is determined by an operation of an engineering scheme on the primary sequence of the parent protein, with the caveat that (i) the at least one portion of the primary sequence of the engineered protein that is determined by the operation of an engineering scheme on the primary sequence of the parent protein comprise at least five percent but does not exceed fifty percent of the length of the primary sequence of the engineered protein.

In some embodiments, the parent protein that has a rubredoxin-like fold has a three-dimensional structure that is approximately ellipsoidal and that comprises a three-stranded antiparallel β-sheet with a hydrophobic core that comprises a plurality of residues (e.g. between four and six hydrophobic residues). In some embodiments, the parent protein that has a rubredoxin-like fold has an alanine residue at a position n, a tryptophan at a position n+2, a glutamic acid at position N+13, and a phenylalanine at a position N+28.

In some embodiments, the parent protein that has a rubredoxin-like fold is a member of the rubredoxin-like superfamily and/or the rubredoxin-like family. The rubredoxin-like superfamily is a superfamily found in the Structural Classification of Protein (SCOP) database (Murzin et al., 1995, J. Mol. Biol. 247, pp. 536-540). The rubredoxin-like superfamily includes all those proteins that are in the rubredoxin family, the desulforedoxin family, and the cytochrome c oxidase subunit F family. Members of the rubredoxin family are discussed in further detail below. Members of the desulforedoxin family include, but are not limited to, desulforedoxin from Desulfovibrio Gigas (Archer et al., 1995, J.Mol.Biol. 251, p. 690), Desulfoferrodoxin from Desulfovibrio Desufuricans (Coelho, 1997, J.Biol. Inorg.Chem. 2, p. 507). Members of the cytochrome c oxidase subunit F family include, but are not limited to Bovine Heart Cytochrome C Oxidase, (Yoshikawa, 1998, Science 280, p. 1723)

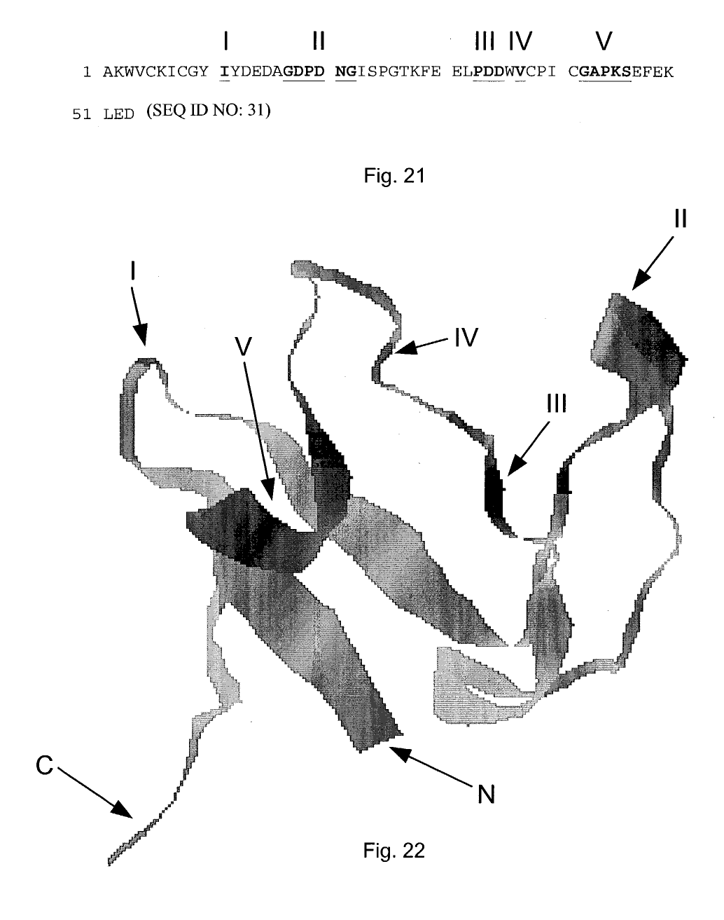

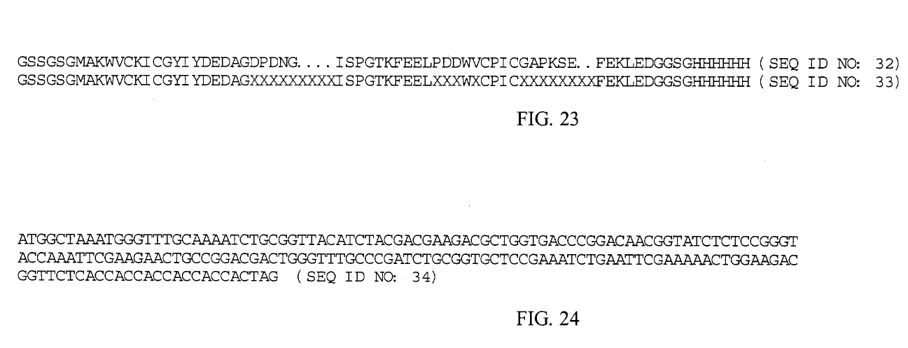

In some embodiments, the parent protein that has a rubredoxin-like fold is a member of the rubredoxin family. The rubredoxin family includes rubredoxins and rubrerythrins. The rubrerythrins include, but are not limited to, rubrerythrin from Desulfovibrio vulgaris (Sieker, 2000, J.Biol.Inorg.Chem. 5, p. 505). The rubredoxins include, but are not limited to rubredoxin from Desulfovibrio vulgaris (Dauter et al., 1992, Acta Crystallogr., Sect.B 48, p. 42); rubredoxin from Desulfovibrio gigas (Frey et al., 1987, J.Mol.Biol. 197, P. 525); rubredoxin from Desulfovibrio desulfuricans (Sicker et al., 1986, Febs Lett. 208, p. 73); rubredoxin from Clostridium pasteurianum (Dauter et al., 1996, Proc. Nat. Acad. Sci. USA 93, 8836); rubredoxin from Pyrococcus Furiosus (FIGS. 21 and 22) (Bau et al., 1998, J. Biol. Inorg. Chem. 3, p. 484); and rubredoxin from Guillardia theta (Schweimer et al., 2001, Protein Sci. 9, p. 1474).

The engineering scheme used in some embodiments of the present invention is randomization. Techniques for randomizing portions of the primary sequence of a protein are known in the art. In one embodiment randomization is effected by constructing a library of engineered proteins from synthetic DNA oligonucleotides by mutually primed extension of the DNA oligonucleotides. Certain positions in these oligonucleotides have degenerate positions that correspond to the regions of the primary sequence of the parent protein that is randomized to provide the resulting library of engineered proteins.

Some embodiments of the present invention provide libraries of engineered proteins in which each engineered protein in the library includes rubredoxin from the hyperthermophilic archeon Pyrococcus furiosus (SEQ ID NO: 31). At least one portion of the primary sequence of this rubredoxin is subjected to an engineering scheme such as randomization. The portion or portions of rubredoxin that are engineered in this embodiment include any combination of the following: (i) a segment comprising isoleucine 11 of SEQ ID NO: 31; (ii) a segment comprising residues glycine 17 through glycine 22 of SEQ ID NO: 31; (iii) a segment comprising proline 33 through aspartic acid 35 of SEQ ID NO: 31 and (iv) a segment comprising valine 37 of SEQ ID NO: 31; and (v) a segment comprising glycine 42 through serine 46 of SEQ ID NO: 31.

6.2.6 Change in Sequence Length of Engineered Proteins Relative to Parent Protein

In some embodiments of the present invention, the randomization of at least one portion of the primary sequence of the parent protein to yield engineered proteins results in a change in the overall number of residues present in the engineered protein domain relative to the number of residues that occur in the parent protein domain. As a non-limiting example, if a parent protein or a protein domain that is used as a basis for engineered proteins has X number of residues, randomization of at least one portion of the primary sequence of the parent protein results in engineered proteins that have X−20 residues, X−15 residues, X−10 residues, X−5 residues, X+5 residues, X+10 residues, X+15 residues, or X+20 residues. Thus, the randomization of the present invention does not preclude deletion schemes in which one or more residues in the parent scaffold are deleted in order to form the engineered proteins. Further, the randomization of the present invention does not preclude insertion schemes in which additional residues are inserted into the one or more randomized portions of the protein scaffold in order to form engineered proteins of the present invention.

6.2.7 Stability of Engineered Proteins

The engineered proteins of the present invention are advantageous in that they are stable enough to use in screening technologies in which the proteins are immobilized on addressable arrays or on beads. Addressable arrays include protein microarrays that are discussed in more detail below. Because of the stability of the engineered proteins in accordance with one embodiment of the present invention, addressable arrays or beads can be stored at room temperature for long periods of time. One embodiment of the present invention provides engineered proteins that are free of disulfide bonds. One example of engineered proteins that are free of disulfide bonds is mutants of the substrate-binding domain of the α subunit of the Thermoplasma acidophilum thermosome (residue 214 to residue 365 of SEQ ID NO: 1). Another example of engineered proteins that are free of disulfide bonds is mutants of rubredoxin from the hyperthermophilic archeon Pyrococcus furiosus (SEQ ID NO: 31).

6.2.8 Solvent Accessibility of Parent Protein Regions that are Subjected to Engineering

One embodiment of the present invention provides engineered proteins. In one embodiment, the parent protein corresponding to these engineered proteins includes a three-layer swiveling β/β/α domain. The central beta sheet of this three-layer domain is parallel and the other beta sheet is antiparallel. In another embodiment, the parent protein is rubredoxin. In another embodiment, the parent protein corresponding to these engineered proteins includes a protein with a rubredoxin-like fold. The rubredoxin-like fold is characterized by a zinc-bound or iron-bound fold by a protein whose primary amino acid sequence comprises two CXₙC motifs, where X is any residue (e.g., a residue of a naturally occurring amino acid) and n is 1, 2, 3, or 4, and in most case n is 2.

At least one portion of the primary sequence of the engineered protein is determined by applying an engineering scheme to a portion of the primary sequence of the parent protein. This engineering scheme may be a randomization scheme. Each such portion of the primary sequence of the parent protein provides a solvent-exposed region of the parent protein. In one embodiment, a residue is considered solvent-exposed if over twenty percent of the surface area of the residue is contacted by a 1.4 Angstrom test sphere as described by Connolly. (See Connolly, Science 221, pp. 709-713, 1983). Thus, in one embodiment, a solvent-exposed region of the parent protein is defined as a region in which at least thirty-five percent of the atoms in the region are solvent-exposed when the parent protein adopts a folded state. In another embodiment, a solvent-exposed region of a protein is defined as a region in which at least fifty percent of the atoms in the region are solvent-exposed when the parent protein adopts a folded state. In yet another embodiment, a solvent-exposed region of the parent protein is defined as a region in which at least sixty-five percent of the atoms in the region are solvent-exposed when the parent protein adopts a folded state.

6.2.9 Method of Making Engineered Proteins

One embodiment of the present invention provides a method of making an engineered protein. The method comprises subjecting at least one portion of the primary sequence of a parent protein to an engineering scheme in order to produce the engineered protein.

In one embodiment, the parent protein comprises a three-layer swiveling β/β/α domain. The central beta sheet of the three-layer swiveling β/β/α domain is parallel and the other beta sheet in the three-layer swiveling β/β/α domain is antiparallel. In some embodiments, the parent protein has a rubredoxin-like fold. The rubredoxin-like fold is a zinc-bound fold or an iron-bound fold adopted by a protein whose primary amino acid sequence includes two CXₙC motifs, where C is cysteine, X is any amino acid, and n is 1, 2, 3, or 4.

The total length of the primary sequence that is determined by the engineering scheme is subject to limitation. The at least one portion of the primary sequence of the engineered protein does not exceed thirty-five percent, forty percent, forty-five percent, fifty percent, fifty-five percent, sixty percent, sixty-five percent, seventy percent, or seventy-five percent of the length of the primary sequence of the engineered protein. Further, the at least one portion of the primary sequence of the engineered protein comprises at least five percent, ten percent, fifteen percent, twenty percent, twenty-five percent, thirty percent, thirty-five percent, or forty percent of the length of the primary sequence of the engineered protein.

In some embodiments, the engineering scheme is a randomization scheme and the step of subjecting the at least one portion of the primary sequence of the parent protein to the engineering scheme results in the randomization of the at least one portion of the primary sequence. In some embodiments, the engineering scheme is a pseudo-randomization scheme and the step of subjecting the at least one portion of the primary sequence of the parent protein to an engineering scheme results in the pseudo-randomization of the at least one portion of the primary sequence.

### 6.3 Engineered Fusion Protein

In some embodiments, the engineered proteins of the present invention are fused to other protein domains derived from publicly available gene sequences and/or commercially available kits. In one embodiment of the present invention, the engineered proteins are fused to a GST, MBP, NusA, or a thioredoxin domain to provide the engineered protein with additional solubility. In some embodiments, the engineered proteins of the present invention are fused to an affinity tag using N-terminal or C-terminal chemistry.

The fusion proteins of the present invention can be produced by standard recombinant DNA techniques. In another embodiment, the fusion gene can be synthesized by conventional techniques including automated DNA synthesis. Alternatively, PCR amplification of gene fragments can be carried out using anchor primers that give rise to complementary overhangs between consecutive gene fragments. The consecutive gene fragments are subsequently annealed and re-amplified to generate a chimeric gene sequence (see, e.g., Current Protocols in Molecular Biology, Ausubel et al., eds., John Wiley & Sons, 1992). Moreover, many expression vectors are commercially available that already encode a fusion moiety (e.g., a GST polypeptide). A nucleic acid encoding an engineered protein of the present invention can be cloned into such an expression vector so that the fusion moiety is linked in-frame to the polypeptide of the invention.

### 6.4 Physiologically Acceptable Carriers

Some of the engineered proteins of the present invention and/or compounds that bind to the engineered proteins of the present invention serve as pharmaceutical compositions. Pharmaceutical compositions for use in accordance with the present invention e.g. methods to treat or prevent harmful diseases, can be formulated in a conventional manner using one or more physiologically acceptable carriers or excipients. Thus, the compounds and their physiologically acceptable salts and solvents can be formulated for administration by inhalation or insufflation (either through the mouth or the nose) or oral, buccal, parenteral or rectal administration. For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound. For buccal administration the compositions can take the form of tablets or lozenges formulated in conventional manner. For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of e.g. gelatin for use in an inhaler or insufflator can be formulated containing a powder mix of the compound and a suitable powder base such as lactose or starch.

The compounds can be formulated for parenteral administration (i.e., intravenous or intramuscular) by injection, via, for example, bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use. The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

### 6.5 Binding Compounds of Interest

One aspect of the present invention provides novel engineered proteins. In some embodiments, the parent protein corresponding to these novel engineered proteins comprises a three-layer swiveling β/β/α domain in which the central beta sheet is parallel and the other beta sheet is antiparallel. In other embodiments, the parent protein corresponding to these novel engineered proteins has a rubredoxin-like fold. The rubredoxin-like fold is a zinc-bound fold or an iron-bound fold adopted by a protein whose primary amino acid sequence includes two CXₙC motifs, where C is cysteine, X is any amino acid, and n is 1, 2, 3, or 4.

In one embodiment of the invention, at least one portion of the primary sequence of each engineered protein is determined by a randomization scheme, such as the exemplary randomization scheme set forth in the examples section below. In this embodiment, the novel mutant proteins are characterized by their ability to bind to a compound that the corresponding parent protein does not specifically bind. A compound as used herein refers to a wide range of molecular entities, including, but not limited to, proteins, hormones, low molecular weight compounds, peptides and oligonucleotides.

6.5.1 Low Molecular Weight Compounds

Low molecular weight compounds include any compound having a molecular weight of less than 2000 Daltons. However, it will be appreciated that compounds that have a molecular weight greater than 2000 Daltons are also within the scope of the present invention if they bind to one of the engineered proteins of the present invention. Representative low molecular weight compounds include organic compounds having a molecular weight of less than 2000 Daltons. Such compounds typically include the atom types O (oxygen), N (nitrogen), S (sulfur), C (carbon), M (metal), and P (phosphorous), and H (hydrogen). The metal atoms (M) include any metallic atom that is from the s-block, p-block or d-block of the periodic table. See, e.g., A Dictionary of Chemistry, Oxford, Great Britain, 1996. The d-block is defined as those elements in Groups IIIB, IVB, VB, VIIB, VIIIB, IB, and IIB of the periodic table. See, e.g., Huheey, Inorganic Chemistry, Harper & Row, New York, 1983. Furthermore, the metal atoms of the present invention may be in any chemically possible oxidation state including, but not limited to, oxidation states zero, one, two, three or four and those that are formally negative. In addition, the metal atoms (M) of the present invention include any isotope of any metal.

Low molecular weight compounds include molecular entities that are characterized as alkyls, substituted alkyls, alkenyls, substituted alkenyls, cycloalkyls, substituted cycloalkyls, heterocycloalkyls, substituted heterocycloalkyls, aryls, alkaryls, heteroaryls, alkheteroaryls, acyl halides, alcohols, aldehydes, amide, amines, arenes, azides, carboxylic acides, esters, ethers, halides, ketones, nitriles, ntiro compounds, phenols, sulfides, sulfones, sulfonic acids, sulfoxides and/or thiols. By way of example only, alkyls are saturated branched, straight chain or cyclic hydrocarbon radicals. Typical alkyl groups include, but are not limited to, methyl, ethyl, propyl, isopropyl, cyclopropyl, butyl, isobutyl, t-butyl, cyclobutyl, pentyl, isopentyl, cyclopentyl, hexyl, cyclohexyl and the, like. Substituted alkyls are alkyl radicals in which one or more hydrogen atoms are each independently replaced with another substituent. Typical substituents include, but are not limited to, —R, —OR, —SR, —NRR, —CN, —NO₂, —C(O)R, —C(O)OR, —C(O)NRR, —C(NRR)═NR, —C(O)NROR, —C(NRR)═NOR, —NR—C(O)R, -tetrazol-5-yl, —NR—SO₂—R, —NR—C(O)—NRR, —NR—C(O)—OR, -halogen and -trihalomethyl where each R is independently —H, (C₁-C₂₀) alkyl, (C₂-C₂₀) alkenyl, (C₂-C₂₀) alkynyl, (C₅-C₂₀) aryl, and (C₆-C₂₆) alkaryl.

Low molecular weight compounds include those molecular entities having one or more aryls or heteroraryls. Aryls are unsaturated cyclic hydrocarbon radicals having a conjugated π electron system. Typical aryl groups include, but are not limited to, penta-2,4-dienyl, phenyl, naphthyl, aceanthrylyl, acenaphthyl, anthracyl, azulenyl, chrysenyl, indacenyl, indanyl, ovalenyl, perylenyl, phenanthrenyl, phenalenyl, picenyl, pyrenyl, pyranthrenyl, rubicenyl and the like. In a preferred embodiment, the aryl group is (C₅-C₂₀) aryl, more preferably (C₅-C₁₀) aryl and most preferably phenyl. Heteroaryls are aryl moieties wherein one or more carbon atoms have been replaced with another atom, such as N, P, O, S, As, Ge, Se, Si, Te, etc. Typical heteroaryl groups include, but are not limited to, acridarsine, acridine, arsanthridine, arsindole, arsindoline, benzodioxole, benzothiadiazole, carbazole, β-carboline, chromane, chromene, cinnoline, furan, imidazole, indazole, indole, isoindole, indolizine, isoarsindole, isoarsinoline, isobenzofuran, isochromane, isochromene. isoindole, isophosphoindole, isophosphinoline, isoquinoline, isothiazole, isoxazole, naphthyridine, perimidine, phenanthridine, phenanthroline, phenazine, phosphoindole, phosphinoline, phthalazine, piazthiole, pteridine, purine, pyran, pyrazine, pyrazole, pyridazine, pyridine, pyrimidine, pyrrole, pyrrolizine, quinazoline, quinoline, quinolizine, quinoxaline, selenophene, tellurophene, thiazopyrrolizine, thiophene and xanthene. In some embodiments, a low molecular weight compound comprises a peptide or protein having a molecular weight of 10 kD or less, a hormone, and/or an oligonucleotide.

6.5.2 Binding Constants

Some embodiments of the present invention provide one or more engineered proteins that specifically bind to a compound that does not specifically bind to the parent protein. More specifically, one embodiment of the present invention provides one or more engineered proteins that each have an EC₅₀ for a compound that is less than 1 millimolar. Furthermore, the parent protein corresponding to the one or more engineered proteins has an EC₅₀ for the compound that is greater than 1 millimolar. Another embodiment of the present invention provides one or more engineered proteins that each have an EC₅₀ for a compound that is less than 1 micromolar. Furthermore, the parent protein corresponding to the one or more engineered proteins has an EC₅₀ for the compound that is greater than 1 micromolar. Yet another embodiment of the present invention provides one or more engineered proteins that have an EC₅₀ for a compound that is less than 100 nM. The parent protein corresponding to the one or more engineered proteins has an EC₅₀ for the compound that is greater than 100 nM.

In one embodiment, a protein binds to a compound when the protein has an EC₅₀ constant for the compound that is less than 1 millimolar. In another embodiment, a protein specifically binds to a compound when the protein has an EC₅₀ for the compound that is less than 1 micromolar. In still another embodiment, a protein specifically binds to a compound when the protein has an EC₅₀ for the compound that is less than 100 nM.

6.5.3 Method for Detecting a Compound in a Sample

One embodiment of the present invention provides a method for detecting a compound in a sample. The method comprises contacting the sample with an engineered protein that specifically binds to the compound.

In some embodiments, the parent protein of the engineered protein comprises a three-layer swiveling β/β/α domain. The central beta sheet of the three-layer swiveling β/β/α domain is parallel and the other beta sheet in the three-layer swiveling β/β/α domain is antiparallel. In some embodiments, the parent protein has a rubredoxin-like fold. The rubredoxin-like fold is a zinc-bound fold or an iron-bound fold adopted by a protein whose primary amino acid sequence includes two CXₙC motifs, where C is cysteine, X is any amino acid, and n is 1, 2, 3, or 4.

Furthermore, at least one portion of the primary sequence of the engineered protein is determined by an operation of an engineering scheme on the primary sequence of the parent protein. However, the total length of the at least one portion of the primary sequence of the engineered protein that is determined by an operation of the engineering scheme is subject to constraints.

The at least one portion of the primary sequence of the engineered protein does not exceed thirty-five percent, forty percent, forty-five percent, fifty percent, fifty-five percent, sixty percent, sixty-five percent, seventy percent, or seventy-five percent of the length of the primary sequence of the engineered protein. Further, the at least one portion of the primary sequence of the engineered protein comprises at least five percent, ten percent, fifteen percent, twenty percent, twenty-five percent, thirty percent, thirty-five percent, or forty percent of the length of the primary sequence of the engineered protein.

In some embodiments, the method further comprises detecting a complex between the engineered protein and the compound. In some embodiments, the parent domain comprises the substrate-binding domain of the α or β subunit of a chaperonin. In some embodiments, the parent domain comprises rubredoxin. In some embodiments, the parent domain is rubredoxin from Desulfovibrio vulgaris (Dauter et al., 1992, Acta Crystallogr., Sect.B 48, p. 42); rubredoxin from Desulfovibrio gigas (Frey et al., 1987, J.Mol.Biol. 197, P. 525); rubredoxin from Desulfovibrio desulfuricans (Sicker et al., 1986, Febs Lett. 208, p. 73); rubredoxin from Clostridium pasteurianum (Dauter et al., 1996, Proc. Nat. Acad. Sci. USA 93, 8836); rubredoxin from Pyrococcus Furiosus (Bau et al., 1998, J. Biol. Inorg. Chem. 3, p. 484); or rubredoxin from Guillardia theta (Schweimer et al., 2001, Protein Sci. 9, p. 1474).

In some embodiments, the sample is a biological sample. In still other embodiments, the engineered protein is immobilized on a bead or a chip. In yet other embodiments, the engineered protein is immobilized on the solid support as part of an array of proteins. In some embodiments the compound is a protein. In other embodiments, the compound is a compound disclosed in Section 5.1. In still other embodiments, the parent domain comprises a Group II chaperonin. In other embodiments, the parent domain comprises a portion of a Thermoplasma acidophilum thermosome. In yet other embodiments, the parent protein comprises Ser 214 through Asn 365 of the α subunit of Thermoplasma acidophilum thermosome (residue 214 through residue 365 of SEQ ID NO: 1) and the at least one portion of the primary sequence of the engineered protein that is determined by an engineering scheme includes any combination of: a segment comprising residue 219 (Asp 219) through residue 226 (Lys 226) of SEQ ID NO: 1; a segment comprising residue 291 (Gln 291) through residue 300 (His 300) of SEQ ID NO: 1; a segment comprising residue 311 (Arg 311) through 315 (Lys 315) of SEQ ID NO: 1; and a segment comprising residue 351 (Lys 351) through residue 357 (Met 357) of SEQ ID NO: 1.

In some embodiments, a complex between the engineered protein and the compound is formed. This complex is detected by methods that include, but are not limited to, spectroscopy, radiography, fluorescence detection, mass spectrometry, luminescence, or surface plasmon resonance. In some embodiments, the dissociation constant of the complex is less than 10⁻⁶ moles/liter.

### 6.6 Attachment of Engineered Proteins to Surfaces

6.6.1 Attachment Chemistry

In some embodiments of the present invention, the engineered proteins are attached to a surface using N-terminal or C-terminal chemistry. Representative surfaces include the arrays disclosed below as well as slides, beads, and other conventional surfaces that are used to present proteins. In one embodiment, free-engineered proteins that specifically bind to a compound retain this compound specificity even after the protein has been attached to a surface.

Some engineered proteins of the present invention include a serine residue or a threonine residue at the extreme N-terminus of the protein. In cases where the corresponding parent protein does not have an N-terminal serine or threonine residue, a serine or threonine is added to the N-terminus of the engineered protein. Proteins are normally expressed in biological systems, such as in bacteria, with a methionine residue at the extreme N-terminus. This methionine is often cleaved off during expression by a specific endoprotease present in bacteria. Therefore, standard molecular biology techniques are used to add a serine residue or a threonine residue the engineered protein; placing it at the second position from the N-terminus, i.e., immediately after the methionine residue at the extreme N-terminus. In alternate embodiments, the recombinant engineered proteins is expressed with an N-terminal sequence that includes a cleavage site for a sequence-specific endoproteases such as enterokinase, factor X, thrombin, etc., followed by a serine or threonine residue, such that cleavage of the cleavage site reveals a new N-terminus bearing a serine or threonine at the extreme N-terminus. Similarly, a recombinant protein can be expressed with a membrane translocation signal at its N-terminus, immediately followed by a serine or threonine residue. In one of various in vivo expression systems, such as bacteria, the encoded protein is translocated across a membrane, after which it is cleaved by a protease present in the compartment into which it is translocated, resulting in an engineered protein with an N-terminal serine or threonine. Regardless of the method used to create an engineered protein with an N-terminal serine or threonine residue, the N-terminal serine or threonine residue can be selectively oxidized to form a glyoxylyl or keto group. The glyoxylyl or keto group is then reacted with a surface functionality. In one example, the surface functionality is an aminooxy or hydrazine functionality. In another example, the surface functionality is provided by a heterobifunctional compound that bears both an aminooxy or hydrazine functionality and a second reactive group that attaches to the surface. In another example, the engineered protein bearing an N-terminal glyoxylyl or keto group is reacted with a biotin derivative bearing an aminooxy or hydrazine functionality, resulting in an N-terminally biotinylated protein. This biotinylated protein is then attached to a surface derivatized with a biotin-binding protein, such as, but not restricted to, avidin, streptavidin, or neutravidin. A non-limiting example of a useful derivative of biotin that includes an aminooxy functionality is N-(aminooxyacetyl)-N′-(D-biotinoyl)hydrazine.

Another embodiment of the present invention provides engineered proteins that include an N-terminal cysteine residue. For reasons discussed above, proteins are not normally expressed with N-terminal cysteine residues, but methods similar to those described above can be used to create recombinant proteins with N-terminal cysteine residues. The engineered protein is attached to a surface by selectively derivatizing the N-terminal cysteine residue with a surface bearing a thioester functionality. The engineered protein will then react with the surface-attached thioester in a transthioesterification reaction. The resulting reaction product will then spontaneously rearrange to form an amide bond between the engineered protein and the surface-attached functionality. In another example, the surface functionality is provided by a heterobifunctional compound that bears both a thioester functionality and a second reactive group that attaches to the surface. In another example, the engineered protein bearing an N-terminal cysteine residue is reacted with a biotin derivative bearing a thioester, resulting in an N-terminally biotinylated protein. This biotinylated protein is then attached to a surface derivatized with a biotin-binding protein such as, but not limited to, avidin, streptavidin, or neutravidin. The natural carboxyl group of biotin could be readily converted to a thioester using standard organic synthesis methods.

6.6.2 Engineered Chaperonin Mutants Arrayed on a Solid Support

One embodiment of the present invention provides an array of engineered proteins immobilized on a solid support, such as a bead, slide, or chip. Each engineered protein in the array comprises an engineered chaperonin domain. Each engineered chaperonin protein is derived from a parent chaperonin domain. To make each engineered protein in the array, one or more portions of the parent chaperonin domain is subjected to an engineering scheme, such as a randomization scheme. When a randomization scheme is used, at least one portion of the primary sequence of each engineered protein in the array of engineered proteins is determined by a randomization scheme. In one embodiment, at least one engineered protein in the array of engineered proteins is characterized by an ability to bind to a compound that the corresponding parent chaperonin domain does not specifically bind. The compound may be a protein, a hormone, a low molecular weight compound, a peptide or an oligonucleotide. In another embodiment, each engineered protein in the array of engineered proteins is a mutant of the substrate-binding domain of a Group II chaperonin. In yet another embodiment, each engineered protein in the array of engineered proteins is derived from the substrate-binding domain of the α or β subunit of the Thermoplasma acidophilum thermosome using an engineering scheme. In still another embodiment, each engineered protein in the array of engineered proteins is derived from residue Ser 214 through residue Asn 365 of the α subunit of the Thermoplasma acidophilum thermosome (residue 214 through residue 365 of SEQ ID NO: 1) and at least one of the following portions of the primary sequence of the α subunit of the Thermoplasma acidophilum thermosome is subjected to an engineering scheme:

a segment comprising residue 219 (Asp 219) through residue 226 (Lys 226) of SEQ ID NO: 1;

a segment comprising residue 291 (Gln 291) through residue 296 (Asp 296) of SEQ ID NO: 1;

a segment comprising residue 311 (Arg 311) through residue 315 (Lys 315) of SEQ ID NO: 1; and

a segment comprising residue 351 (Lys 351) through residue 357 (Met 357) of SEQ ID NO: 1.

6.6.3 Engineered Rubredoxin Mutants Arrayed on a Solid Support

One embodiment of the present invention provides an array of engineered proteins immobilized on a solid support, such as a bead, slide, or chip. Each engineered protein in the array is made by engineering a parent protein that has a rubredoxin-like fold. To make each engineered protein in the array, one or more portions of the parent protein is subjected to an engineering scheme, such as a randomization scheme. When a randomization scheme is used, at least one portion of the primary sequence of each engineered protein in the array of engineered proteins is determined by a randomization scheme. In one embodiment, at least one engineered protein in the array of engineered proteins is characterized by an ability to bind to a compound that the corresponding parent protein (the protein with a rubredoxin-like fold) does not bind. The compound may be a protein, a hormone, a low molecular weight compound, a peptide or an oligonucleotide. In another embodiment, each engineered protein in the array of engineered proteins is a mutant of rubredoxin from Pyrococcus furiousus, Desulfovibrio gigas, Pseudomonas oleovorans, Clostridium pasteurianum, Desulfovibrio vulgaris, Desulfovibrio desulfuricans, or Guillardia theta.

In another embodiment, each engineered protein in the array of engineered proteins is derived from Pyrococcus furious rubredoxin (SEQ ID NO: 31) and at least one of the following portions of the primary sequence of this parent protein is subjected to an engineering scheme:

(i) a segment comprising isoleucine 11 of SEQ ID NO: 31;

(ii) a segment comprising glycine 17 through glycine 22 of SEQ ID NO: 31;

(iii) a segment comprising proline 33 through aspartic acid 35 of SEQ ID NO: 31;

(iv) a segment comprising valine 37 of SEQ ID NO: 31; and

(v) a segment comprising glycine 42 through serine 46 of SEQ ID NO: 31.

6.6.4 Surfaces Used to Attach Engineered Proteins of the Present Invention

6.6.4.1 Protein Patches on Substrates

Overview. In one embodiment, the arrays of engineered proteins of the present invention comprise micrometer-scale, two-dimensional patterns of patches of engineered proteins immobilized on an organic thinfilm coating on the surface of a substrate. Additional description of arrays in accordance with this embodiment of the invention is found in Wagner et al. WO 00/04382 A1, and Wagner et al., U.S. Pat. No. 6,329,209, which is a continuation in part of U.S. patent application Ser. No. 09/115,455, filed Jul. 14, 1998.

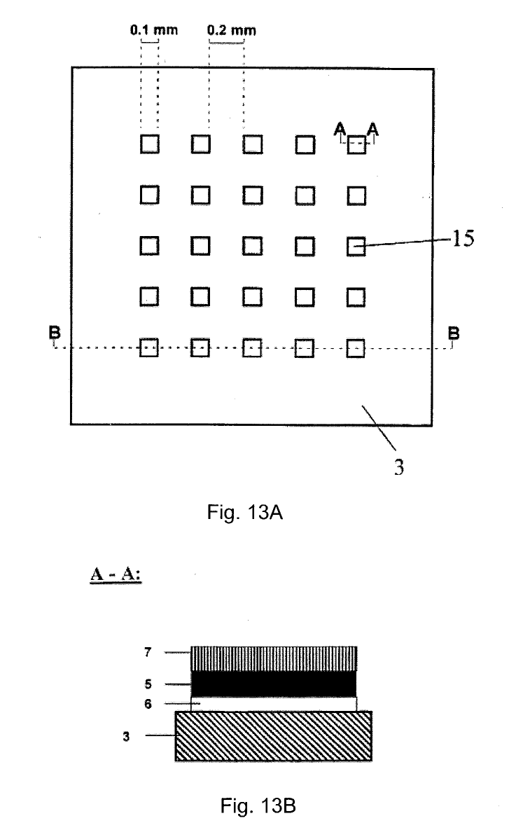

FIG. 13A shows the top view of one example of an array in accordance with this embodiment of the present invention. On the array, a number of patches 15 cover the surface of the substrate 3. FIG. 13B shows a detailed cross section of a patch 15 of the array of FIG. 13A. FIG. 13B illustrates the use of a coating 5 on the substrate 3. The term “coating” means a layer that is either naturally or synthetically formed on or applied to the substrate surface. In an embodiment, the coating is derived from oxidizing the substrate surface or by deposition via mechanical, physical, electrical, or chemical means. An example of the type of coating that is applied by deposition is a metal coating that is applied to a silicon or polymer substrate or a silicon nitride coating that is applied to a silicon substrate. Although a coating may be of any thickness, typically the coating has a thickness smaller than that of the substrate.

FIG. 13B further illustrates an adhesion interlayer 6 that is included in the patch. On top of the patch resides a self-assembled monolayer 7. FIG. 13C shows a cross section of one row of the patches 15 of the array of FIG. 13A. This figure also shows the use of a cover 2 over the array. Use of the cover 2 creates an inlet port 16 and an outlet port 17 for solutions to be passed over the array.

Patches. Arrays in this aspect of the invention comprise at least ten patches. In some embodiments, the array comprises at least 50 patches. In still other embodiments, the array comprises at least 100 patches, 10³, 10⁴, 10⁵ or more patches. The area of surface of the substrate covered by each patch is preferably no more than 0.25 mm². Preferably, the area of the substrate surface covered by each of the patches is between 1 μm² and 10,000 μm². In one embodiment, each patch covers an area of the substrate surface from 100 μm² to 2,500 μm². In an alternative embodiment, a patch on the array covers an area of the substrate surface as small as 2,500 nm².

The patches of the array may have any geometric shape. For instance, the patches may be rectangular or circular. The patches may also be irregularly shaped. In one embodiment, the patches are elevated from the median plan of the underlying substrate. The distance between each patch of the array can vary. Preferably, the patches of the array are separated from neighboring patches by 1 μm to 500 μm. Typically, the distance separating the patches is roughly proportional to the diameter or side length of the patches on the array if the patches have dimensions greater than 10 μm. If the patch size is smaller, then the distance separating the patches will typically be larger than the dimensions of the patch.

In a preferred embodiment, the patches are encompassed within an area of 1 cm² or less on the surface of the substrate. In one embodiment, therefore, the array comprises 100 or more patches within a total area of 1 cm² or less on the surface of the substrate. Alternatively, a preferred array comprises 10³ or more patches within a total area of 1 cm² or less. A preferred array may even comprise 10⁴ or 10⁵ or more patches within an area of 1 cm² or less on the surface of the substrate. In other embodiments of the invention, all of the patches of the array are enclosed within an area of 1 mm² or less of substrate surface area.

The arrays can have any number of a plurality of engineered proteins. Typically, the array comprises a library of at least ten different engineered proteins. Preferably, the array comprises at least 50 different engineered proteins. More preferably, the array comprises at least 100 different engineered proteins. Alternative preferred arrays comprise more than 10³ different engineered proteins or more than 10⁴ different engineered proteins. The array optionally comprises more than 10⁵ different engineered proteins.

In one embodiment, each of the patches of the array comprises a different engineered protein selected from a library of engineered proteins in which each library member is derived from a parent chaperonin or a protein that has a rubredoxin-like fold (e.g., rubredoxin). For instance, an array comprising 100 patches could comprise 100 different engineered proteins. Likewise, an array of 10,000 patches could comprise 10,000 different engineered proteins. In an alternative embodiment, however, each different engineered protein is immobilized on more than one separate patch on the array. For instance, each different engineered protein is optionally present on two to six different patches. An exemplary array of the present invention, therefore, comprises three-thousand engineered protein patches, but only represents one thousand different engineered proteins since each different engineered protein is present on three different patches.

Substrates, coatings, and organic thinfilms. The substrates used for arrays in accordance with this embodiment of the present invention are either organic or inorganic, biological or non-biological, or any combination of such materials. In one embodiment, the substrate is transparent or translucent. The portion of the surface of the substrate on which the patches reside is preferably flat and firm or semi-firm. However, the arrays in accordance with this embodiment of the present invention need not be flat. Significant topological features may be present on the surface of the substrate surrounding the patches, between the patches or beneath the patches. For instance, walls or other barriers may separate the patches of the array.

Numerous materials are suitable for use as a substrate in the arrays in accordance with this embodiment of the invention. For instance, the substrate can comprise a material selected from a group consisting of silicon, silica, quartz, glass, controlled pore glass, carbon, alumina, titania, tantalum oxide, germanium, silicon nitride, zeolites, and gallium arsenide. Many metals, such as gold, platinum, aluminum, copper, titanium, and their alloys, are also options for substrates of the array. In addition, many ceramics and polymers may also be used as substrates. Polymers that may be used as substrates include, but are not limited to polystyrene, poly(tetra)fluoroethylene (PTFE), polyvinylidenedifluoride, polycarbonate, polymethylmethacrylate, polyvinylethylene, polyethyleneimine, poly(etherether)ketone, polyoxymethylene (POM), polyvinylphenol, polylactides, polymethacrylimide (PMI), polyalkenesulfone (PAS), polypropylethylene, polyethylene, polyhydroxyethylmethacrylate (HEMA), polydimethylsiloxane, polyacrylamide, polyimide, and block-copolymers. Preferred substrates for the array include silicon, silica, glass, and polymers. The substrate on which the patches reside may also be any combination of substrate materials.

Arrays in accordance with this embodiment of the invention optionally further comprise a coating. This coating is either formed on the substrate or applied to the substrate. The substrate can be modified with a coating by using thin-film technology based, for instance, on physical vapor deposition (PVD), plasma-enhanced chemical vapor deposition (PECVD), or thermal processing. Alternatively, plasma exposure can be used to directly activate or alter the substrate and create a coating. For instance, plasma etch procedures can be used to oxidize a polymeric surface which then acts as a coating.

The coating is optionally a metal film. Possible metal films include aluminum, chromium, titanium, tantalum, nickel, stainless steel, zinc, lead, iron, copper, magnesium, manganese, cadmium, tungsten, cobalt, and alloys or oxides thereof. In a preferred embodiment, the metal film is a noble metal film. Noble metals that used for a coating include, but are not limited to, gold, platinum, silver, and copper. In an especially preferred embodiment, the coating comprises gold or a gold alloy. Electron-beam evaporation may be used to provide a thin coating of gold on the surface of the substrate. In a preferred embodiment, the metal film is from 50 nM to 500 nM in thickness. In an alternative embodiment, the metal film is from 1 nM to 1 μM in thickness. In alternative embodiments, the coating comprises a composition selected from the group consisting of silicon, silicon oxide, titania, tantalum oxide, silicon nitride, silicon hydride, indium tin oxide, magnesium oxide, alumina, glass, hydroxylated surfaces, and polymers.

In one embodiment of the invention, the surface of the coating is atomically flat. In this embodiment, the mean roughness of the surface of the coating is less than 5 Angstroms for areas of at least 25 μM². In a preferred embodiment, the mean roughness of the surface of the coating is less than three Angstroms for areas of at least 25 μM². The ultraflat coating can optionally be a template-stripped surface as described in Hegner et al., Surface Science, 1993, 291:39-46 and Wagner et al., Langmuir, 1995, 11:3867-3875.

It is contemplated that the coatings of many arrays will require the addition of at least one adhesion layer between that coating and the substrate. Typically, the adhesion layer will be at least 6 Angstroms thick or more. For instance, a layer of titanium or chromium may be desirable between a silicon wafer and a gold coating. In an alternative embodiment, an epoxy glue such as Epo-tek 377®, Epo-tek 301-2®, (Epoxy Technology Inc., Billerica, Mass.) is used to aid adherence of the coating to the substrate. In other embodiments, additional adhesion mediators or interlayers are necessary to improve the optical properties of the array, for instance, waveguides used for detection purposes.

Deposition or formation of the coating (if present) on the substrate is performed prior to the formation of the organic thinfilm. Several different types of coating may be combined-on the surface. The coating covers the whole surface of the substrate or only parts of it. The pattern of the coating does not have to be identical to the pattern of organic thinfilms used to immobilize the engineered proteins. In one embodiment of the invention, the coating covers the substrate surface only at the site of the patches of engineered proteins. Techniques useful for the formation of coated patches on the surface of the substrate that are compatible with organic thinfilms are known. For instance, the patches of coatings on the substrate are optionally fabricated by photolithography, micromolding (PCT Publication WO 96/29629), wet chemical and/or dry etching.

The organic thinfilm forms a layer either on the substrate itself or on a coating covering the substrate. The organic thinfilm is preferably less than 20 nM thick. In some embodiments of the invention, the organic thinfilm of each patch is less than 10 nM thick.

A variety of different organic thinfilms are suitable for use in the present invention. Methods for the formation of organic thinfilms include in situ growth from the surface, deposition by spin-coating, chemisorption, self-assembly, or plasma-initiated polymerization from gas phase. For instance, a hydrogel composed of a material such as dextran can serve as a suitable organic thinfilm on the patches of the array. In one embodiment, the organic thinfilm is a lipid bilayer. In another embodiment, the organic thinfilm of each of the patches of the array is a monolayer of polyarginine or polylysine adsorbed on a negatively charged substrate or coating. Another option is a disordered monolayer of tethered polymer chains. In one embodiment, the organic thinfilm is a self-assembled monolayer. The organic thinfilm is often a self-assembled monolayer that comprises molecules of the formula X—R—Y, where R is a spacer, X is a functional group that binds R to the surface, and Y is a functional group for binding engineered proteins onto the monolayer. In an alternative embodiment, the self-assembled monolayer comprises molecules of the formula (X)ₐR(Y)_{b} where a and b are, independently, integers greater than or equal to 1, and X, R, and Y are as previously defined. In yet another embodiment, the organic thinfilm comprises a combination of organic thinfilms, such as a combination of a lipid bilayer immobilized on top of a self-assembled monolayer of molecules of the formula X—R—Y. In another example, a monolayer of polylysine is optionally combined with a self-assembled monolayer of molecules of the formula X—R—Y (see U.S. Pat. No. 5,629,213).

In one embodiment, the regions of the substrate surface, or coating surface, that separate the patches of engineered proteins are free of organic thinfilm. Alternatively, the organic thinfilm extends beyond the area of the substrate surface, or coating surface if present, covered by the patches of engineered protines. As an example, the entire surface of the array is covered by an organic thinfilm on which the plurality of spatially distinct patches of engineered proteins reside. An organic thinfilm that covers the entire surface of the array is either homogenous or comprises patches of differing exposed functionalities useful in the immobilization of patches of different engineered proteins.

A variety of techniques are used to generate patches of organic thinfilm on the surface of the substrate or on the surface of a coating on the substrate. These techniques vary depending upon the nature of the organic thinfilm, the substrate, and the coating if present. The techniques also vary depending on the structure of the underlying substrate and the pattern of any coating present on the substrate. For instance, patches of a coating that are highly reactive with an organic thinfilm may have already been produced on the substrate surface. Arrays of patches of organic thinfilm can optionally be created by microfluidics printing, microstamping (U.S. Pat. Nos. 5,512,131 and 5,731,152), or microcontact printing (μCP) (PCT Publication WO 96/29629). Subsequent immobilization of the engineered proteins to the reactive monolayer patches results in two-dimensional arrays of the agents. Inkjet printer heads provide another option for patterning monolayer X—R—Y molecules, or components thereof, or other organic thinfilm components to nanometer or micrometer scale sites on the surface of the substrate or coating (Lemmo et al., Anal Chem., 1997, 69:543-551; U.S. Pat. Nos. 5,843,767 and 5,837,860). In some cases, commercially available arrayers based on capillary dispensing (for instance, OmniGrid™ from Genemachines, inc, San Carlos, Calif., and High-Throughput Microarrayer from Intelligent Bio-Instruments, Cambridge, Mass.) may be of use in directing components of organic thinfilms to spatially distinct regions of the array.

Diffusion boundaries between the patches of engineered proteins immobilized on organic thinfilms such as self-assembled monolayers may be integrated as topographic patterns (physical barriers) or surface functionalities with orthogonal wetting behavior (chemical barriers). For instance, walls of substrate material or photoresist may be used to separate some of the patches from some of the others or all of the patches from each other.

In a preferred embodiment of the invention, each of the patches of engineered proteins comprise a self-assembled monolayer of molecules of the formula X—R—Y, as previously defined, and the patches are separated from each other by surfaces free of the monolayer.

A variety of chemical moieties may function as monolayer molecules of the formula X—R—Y in the array of the present invention. However, three major classes of monolayer formation are preferably used to expose high densities of reactive omega-functionalities on the patches of the array: (i) alkylsiloxane monolayers (“silanes”) on hydroxylated and non-hydroxylated surfaces (as taught in, for example, U.S. Pat. No. 5,405,766, PCT Publication WO 96/38726, U.S. Pat. No. 5,412,087, and U.S. Pat. No. 5,688,642), (ii) alkyl-thiol/dialkyldisulfide monolayers on noble metals (preferably Au(111)) (as, for example, described in Allara et al., U.S. Pat. No. 4,690,715; Bamdad et al., U.S. Pat. No. 5,620,850; Wagner et al., Biophysical Journal, 1996, 70:2052-2066), and (iii) alkyl monolayer formation on oxide-free passivated silicon (as taught in, for example, Linford et al., J. Am. Chem. Soc., 1995, 117:3145-3155, Wagner et al., Journal of Structural Biology, 1997, 119:189-201, U.S. Pat. No. 5,429,708). It will be appreciated that many possible moieties can be substituted for X, R, and/or Y, dependent primarily upon the choice of substrate, coating, and affinity tag. Many examples of monolayers are described in Ulman, An Introduction to Ultrathin Organic Films: From Langmuir-Blodgett to Self Assembly, Academic press (1991).

In one embodiment, the monolayer comprises molecules of the formula (X)ₐR(Y)_{b} wherein a and b are, independently, equal to an integer between 1 and 200. In a preferred embodiment, a and b are, independently, equal to an integer between 1 and 80. In a more preferred embodiment, a and b are, independently, equal to 1 or 2. In a most preferred embodiment, a and b are both equal to 1 (molecules of the formula X—R—Y).

If the patches of the invention array comprise a self-assembled monolayer of molecules of the formula (X)ₐR(Y)_{b}, then R optionally comprises a linear or branched hydrocarbon chain from 1 to 400 carbons long. In various embodiments, the hydrocarbon chain comprises an alkyl, aryl, alkenyl, alkynyl, cycloalkyl, alkaryl, aralkyl group, or any combination thereof. If a and b are both equal to one, then R is typically an alkyl chain from 3 to 30 carbons long. In one embodiment, if a and b are both equal to one, then R is an alkyl chain from 8 to 22 carbons long and is, optionally, a straight alkane. However, it is also contemplated that, in an alternative embodiment, R comprises a linear or branched hydrocarbon chain from 2 to 400 carbons long and is interrupted by at least one hetero group. The interrupting hetero groups can include, for example, —O—, —CONH—, —CONHCO—, —NH—, —CSNH—, —CO—, —CS—, —S—, —SO—, —(OCH₂CH₂)ₙ— (where n=1-20), —(CF₂)ₙ— (where n=1-22). Alternatively, one or more of the hydrogen moieties of R is substituted with deuterium. In alternative embodiments, R is more than 400 carbons long.

X is any group that affords chemisorption or physisorption of the monolayer onto the surface of the substrate (or the coating, if present). When the substrate or coating is a metal or metal alloy, X, at least prior to incorporation into the monolayer, can in one embodiment be chosen to be an asymmetrical or symmetrical disulfide, sulfide, diselenide, selenide, thiol, isonitrile, selenol, a trivalent phosphorus compound, isothiocyanate, isocyanate, xanthanate, thiocarbamate, a phosphine, an amine, thio acid or a dithio acid. This embodiment is especially preferred when a coating or substrate that is a noble metal is used.

If the substrate of the array is a material, such as silicon, silicon oxide, indium tin oxide, magnesium oxide, alumina, quartz, glass, or silica, then the array of one embodiment of the invention comprises an X that, prior to incorporation into the monolayer, is a monohalosilane, dihalosilane, trihalosilane, trialkoxysilane, dialkoxysilane, or a monoalkoxysilane. Among these silanes, trichlorosilane and trialkoxysilane are particularly preferred.

In a preferred embodiment of the invention, the substrate is selected from the group consisting of silicon, silicon dioxide, indium tin oxide, alumina, glass, and titania. Further X, prior to incorporation into the monolayer, is selected from the group consisting of a monohalosilane, dihalosilane, trihalosilane, trichlorosilane, trialkoxysilane, dialkoxysilane, monoalkoxysilane, carboxylic acids, and phosphates.

In one embodiment, the substrate is silicon and X is an olefin. In another embodiment, the coating (or the substrate if no coating is present) is titania or tantalum oxide and X is a phosphate. In still other embodiments, the surface of the substrate (or coating thereon) is composed of a material such as titanium oxide, tantalum oxide, indium tin oxide, magnesium oxide, or alumina where X is a carboxylic acid or alkylphosphoric acid. Alternatively, if the surface of the substrate (or coating thereon) of the array is copper, then X is optionally a hydroxamic acid.

If the substrate used in the invention is a polymer, then in many cases a coating on the substrate, such as a copper coating, is included in the array. An appropriate functional group X for the coating is then chosen for use in the array. In an alternative embodiment comprising a polymer substrate, the surface of the polymer is plasma-modified to expose desirable surface functionalities for monolayer formation. For instance, EP 780423 describes the use of a monolayer molecule that has an alkene X functionality on a plasma exposed surface. In alternative embodiments, X, prior to incorporation into the monolayer, is a hydroxyl, carboxyl, vinyl, sulfonyl, phosphoryl, silicon hydride, or an amino group.

The component, Y, of the monolayer is a functional group responsible for binding an engineered protein onto the monolayer. In one embodiment, Y is either highly reactive (activated) towards the engineered protein (or its affinity tag) or is easily converted into such an activated form. In a preferred embodiment, the coupling of Y with the engineered protein occurs readily under normal physiological conditions not detrimental to the integrity of the engineered protein. Y either forms a covalent linkage or a noncovalent linkage with the engineered protein (or its affinity tag, if present). In one embodiment, the functional group Y forms a covalent linkage with the engineered protein or its affinity tag.

In one embodiments, Y is a functional group that is activated in situ. Possibilities for this type of functional group include, but are not limited to, moieties such as a hydroxyl, carboxyl, amino, aldehyde, carbonyl, methyl, methylene, alkene, alkyne, carbonate, aryliodide, or a vinyl group. In another embodiment, Y comprises a functional group that requires photoactivation prior to becoming activated enough to trap the engineered protein.

In another embodiment, Y is a highly reactive functional moiety that is compatible with monolayer formation and needs no in situ activation prior to reaction with the engineered protein and/or affinity tag. Such possibilities for Y include, but are not limited to, maleimide, N-hydroxysuccinimide (Wagner et al., Biophysical Journal, 1996, 70:2052-2066), nitrilotriacetic acid (U.S. Pat. No. 5,620,850), activated hydroxyl, haloacetyl, bromoacetyl, iodoacetyl, activated carboxyl, hydrazide, epoxy, aziridine, sulfonylchloride, trifluoromethyldiaziridine, pyridyldisulfide, N-acyl-imidazole, imidazolecarbamate, vinylsulfone, succinimidylcarbonate, arylazide, anhydride, diazoacetate, benzophenone, isothiocyanate, isocyanate, imidoester, fluorobenzene, and biotin. In an alternative embodiment, the functional group Y of the array is —OH, —NH₂, —COOH, —COOR, —RSR, —PO₄⁻³, —OSO₃⁻², —SO₃⁻, —COO⁻, —SOO⁻, —CONR₂, —CN, or —NR₂.

Optionally, the monolayer molecules of the present invention are assembled on the surface in parts. In other words, the monolayer need not be constructed by chemisorption or physisorption of molecules of the formula X—R—Y to the surface of the substrate (or coating). Rather, X is chemisorbed or physisorbed to the surface of the substrate (or coating) first. Then, R, or even just individual components of R, are attached to X through a suitable chemical reaction. Upon completion of addition of the spacer R to the X moiety already immobilized on the surface, Y is attached to the ends of the monolayer molecule through a suitable covalent linkage.

Not all self-assembled monolayer molecules on a given patch need be identical to one another. Some patches comprise mixed monolayers. For instance, the monolayer of an individual patch optionally comprises at least two different molecules of the formula X—R—Y, as previously described. This second X—R—Y molecule is immobilized to the same or a different engineered protein. In addition, some of the monolayer molecules X—R—Y of a patch fail to attach any engineered protein.

As another alternative embodiment of the invention, a mixed, self-assembled monolayer of an individual patch on the array comprises both molecules of the formula X—R—Y, as previously described, and molecules of the formula, X—R—V, where R is a spacer, X is a functional group that binds R to the surface, and V is a moiety that is biocompatible with proteins and resistant to the non-specific binding of proteins. In one example, V consists of a hydroxyl, saccharide, or oligo/polyethylene glycol moiety (EP Publication 780423).

In still another embodiment of the invention, the array comprises at least one unreactive patch of organic thinfilm on the substrate or coating surface that is devoid of any engineered protein. For instance, the unreactive patch optionally comprises a monolayer of molecules of the formula X—R—V, where R is a spacer, X is a functional group that binds R to the surface, and V is a moiety resistant to the non-specific binding of proteins. The unreactive patch may serve as a control patch that is useful in background binding measurements.

Regardless of the nature of the monolayer molecules, in some arrays, it is desirable to provide crosslinking between molecules of the monolayer of an individual patch. In general, crosslinking confers additional stability to the monolayer. Such methods are familiar to those skilled in the art. See, for instance, Ulman, An Introduction to Ultrathin Organic Films: From Langmuir-Blodgett to Self-Assembly, Academic Press (1991).

After completion of formation of the monolayer on the patches, the engineered protein is attached to the monolayer via interaction with the Y-functional group. Y-functional groups that fail to react with any engineered proteins are preferably quenched prior to use of the array.

Affinity tags and immobilization of protein-capture agents. In a one embodiment, the protein-immobilizing patches of the arrays further comprise an affinity tag that enhances immobilization of the engineered protein onto the organic thinfilm. The use of an affinity tag provides several advantages. An affinity tag confers enhanced binding or reaction of the engineered protein with the functionalities on the organic thinfilm, such as Y, if the organic thinfilm is an X—R—Y monolayer as previously described. This enhancement effect may be either kinetic or thermodynamic. The affinity tag/thinfilm combination used in the patches of the array preferably allows for immobilization of the engineered proteins in a manner that does not require reaction conditions that are adverse to protein stability or function. In many embodiments, immobilization to the organic thinfilm in aqueous and biological buffers is preferred.

In a preferred embodiment, the affinity tag comprises at least one amino acid. The affinity tag may be a polypeptide comprising at least two amino acids which is reactive with the functionalities of the organic thinfilm. Alternatively, the affinity tag is a single amino acid that reacts with the organic thinfilm. Examples of possible amino acids that could react with an organic thinfilm include cysteine, lysine, histidine, arginine, tyrosine, aspartic acid, glutamic acid, tryptophan, serine, threonine, and glutamine.

A polypeptide or amino acid affinity tag is preferably expressed as a fusion protein with the engineered protein. Amino acid affinity tags provide either a single amino acid or a series of amino acids that can interact with the functionality of the organic thinfilm, such as the Y-functional group of the self-assembled monolayer molecules. Amino acid affinity tags can be readily introduced into recombinant proteins to facilitate oriented immobilization by covalent binding to the Y-functional group of a monolayer or to a functional group on an alternative organic thinfilm. The affinity tag optionally comprises a poly(amino acid) tag. A poly(amino acid) tag is a polypeptide that has from 2 to 100 residues of a single amino acid, optionally interrupted by residues of other amino acids. For instance, the affinity tag may comprise a poly-cysteine, polylysine, poly-arginine, or poly-histidine. Amino acid tags are preferably composed of two to twenty residues of a single amino acid, such as, for example, histidines, lysines, arginines, cysteines, glutamines, tyrosines, or any combination of these. According to a preferred embodiment, an amino acid tag of one to twenty amino acids includes at least one to ten cysteines for thioether linkage, one to ten lysines for amide linkage, or one to ten arginines for coupling to vicinal dicarbonyl groups.

Affinity tags may contain one or more unnatural amino acids. Unnatural amino acids can be introduced using suppressor tRNAs that recognize stop codons (i.e., amber) (Noren et al., Science, 1989, 244:182-188; Ellman et al., Methods Enzym., 1991, 202:301-336; Cload et al., Chem. Biol., 1996, 3:1033-1038). The tRNAs are chemically amino-acylated to contain chemically altered (“unnatural”) amino acids for use with specific coupling chemistries (i.e., ketone modifications, photoreactive groups). In an alternative embodiment the affinity tag comprise an intact protein, such as, but not limited to, glutathione S-transferase, an antibody, avidin, or streptavidin. In an alternative embodiment of the invention, the organic thinfilm of each of the patches comprises, at least in part, a lipid monolayer or bilayer, and the affinity tag comprises a membrane anchor.

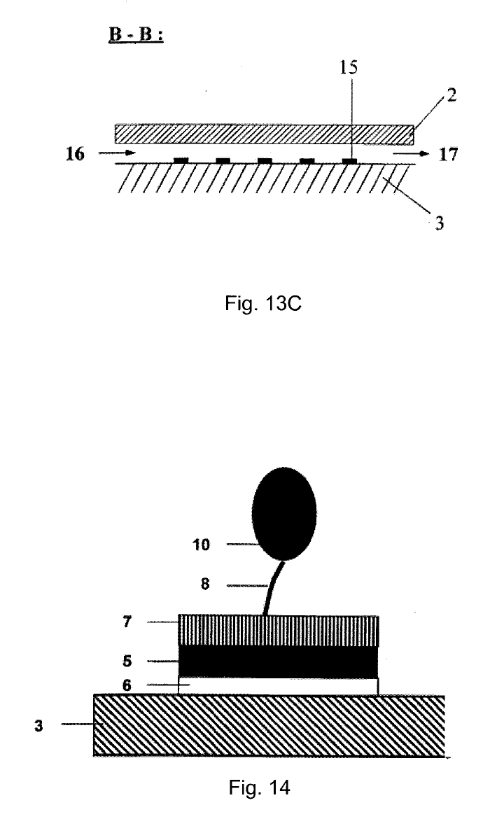

FIG. 14 shows a detailed cross section of a patch on one embodiment of the invention array. In this embodiment, an engineered protein 10 is immobilized on a monolayer 7 on a substrate 3. An affinity tag 8 connects the engineered protein 10 to the monolayer 7. The monolayer 7 is formed on a coating 5 that is separated from substrate 3 by interlayer 6.

Adaptors and Examples. Another embodiment of the array of the present invention comprises an adaptor that links the affinity tag to the engineered protein on the patches of the array. More information on adaptors may be found in Wagner et al. WO/004382. Furthermore, specific examples on how the arrays used in this embodiment of the invention are synthesized are found in Wagner et al. WO/004382, Wagner et al., Biophys. J., 1996, 70:2052-2066, and Wagner, et al., U.S. Pat. No. 6,329,209.

6.6.4.2 Microdevices

General Architecture. In this aspect of the invention, the arrays of engineered proteins of the present invention comprise a plurality of noncontiguous reactive sites, each of which comprises the following: a substrate, an organic thinfilm chemisorbed or physisorbed on a portion of a surface of the substrate, and an engineered protein immobilized on the organic thinfilm. Each of the sites may independently react with a component of a fluid sample. Furthermore, sites are separated from each other by a region of the substrate that is free of the organic thinfilm. Additional description of devices that present arrays in accordance with this aspect of the invention is found in U.S. patent application Ser. No. 09/353,554, filing date Jul. 14, 1999, which is a continuation-in-part of U.S. patent application Ser. No. 09/115,397, filing date Jul. 14, 1998.

In a one embodiment, each of the reactive sites of the device is in a microchannel oriented parallel to microchannels of other reactive sites on the device. The microchannels of such a device are optionally microfabricated or micromachined into the substrate. A reactive site optionally covers the entire interior surface of the microchannel or alternatively, only a portion of the interior surface of the microchannel.

In another embodiment, the invention provides a device for analyzing components of a fluid sample that comprises a substrate, a plurality of parallel microchannels microfabricated into the substrate, and a engineered protein immobilized within at least one of the parallel microchannels. The engineered protein may interact with a component of the fluid sample. Typically, a number of parallel microchannels comprise immobilized engineered proteins.

The dimensions of the microchannels may vary. However, in preferred embodiments, the scale is small enough so as to only require minute fluid sample volumes. The width and depth of each microchannel is typically between 10 mM and 500 mM. In a one embodiment, the width and depth of each microchannel is between 50 mM and 200 mM. The length of each microchannel is from 1 mM to 20 mM in length. In a preferred embodiment, the length of each microchannel is from 2 mM to 8 mM long. Any channel cross-section geometry (trapezoidal, rectangular, v-shaped, semicircular, etc.) may be employed in the device. The geometry is determined by the type of microfabrication or micromachining process used to generate the microchannels, as is known in the art. Trapezoidal or rectangular cross-section geometries are preferred for the microchannels, since they readily accommodate standard fluorescence detection methods.

Substrates. Numerous different materials may be used as the substrate of the invention device. The substrate may be organic or inorganic, biological or non-biological, or any combination of these materials. In fact, any combination of the substrate materials disclosed in section 6.3.1.2 may be used in the substrates in accordance with this aspect of the invention. Preferred substrates for the device include silicon, silica, glass, and polymers.

Substrate cleaning and channel formation. In order to generate a plurality of reactive sites, such as a parallel array of microchannels, the substrate material is cleaned to remove contaminants such as solvent stains, dust, or organic residues. A variety of cleaning procedures are used depending on the substrate material and origin of contaminants. These include wet immersion processes (for example, RCA1+2, “pyranha”, solvents), dry vapor phase cleaning, thermal treatment, plasma or glow discharge techniques, polishing with abrasive compounds, short-wavelength light exposure, ultrasonic agitation and treatment with supercritical fluids.

After cleaning, channels are formed on the surface of the substrate by either (1) bulk micromachining, (2) sacrificial micromachining, (3) LIGA (high aspect ratio plating) or (4) other techniques. Such techniques are well known in the semiconductor and microelectronics industries and are described in, for example, Ghandi, VLSI Fabrication Principles, Wiley (1983) and Sze, VLSI Technology, 2nd. Ed., McGraw-Hill (1988); Wolf and Taube, Silicon Processing for the VLSI Era, Vol. 1, Lattice Press (1986), and Madou, Fundamentals of Microfabrication, CRC Press (1997).

In bulk micromachining, large portions of the substrate are removed to form rectangular or v-shaped grooves comprising the final dimensions of the microchannels. This process is usually carried out with standard photolithographic techniques involving spin-coating of resist materials, illumination through lithography masks followed by wet-chemical development and posttreatment steps such as descumming and post-baking. The resulting resist pattern is then used as an etch resist material for subsequent wet or dry etching of the bulk material to form the desired topographical structures. Typical resist materials include positive and negative organic resists (such as Kodak 747, PR102), inorganic materials (such as polysilicon, silicon nitride) and biological etch resists (for example Langmuir-Blodgett films and two-dimensional protein crystals such as the S-layer of Sulfolobus acidocladarius). Pattern transfer into the substrate and resist stripping occurs via wet-chemical and dry etching techniques including plasma etching, reactive ion etching, sputtering, ion-beam-assisted chemical etching and reactive ion beam etching.

In one embodiment of the invention, for instance, a photoresist is spincoated onto a cleaned 4 inch Si(110) wafer. Ultraviolet light exposure through a photomask onto the photoresist then results in a pattern of channels in the photoresist, exposing a pattern of strips of the silicon underneath. Wet-chemical etching techniques are then be applied to etch the channel pattern into the silicon. Next, a thin layer of titanium can be coated on the surface. A thin layer of gold is then coated on the surface via thermal or electron beam evaporation. Standard resist stripping follows. (Alternatively, the gold-coating could be carried out after the strip resist.)

A cross-sectional view of one example of a microchannel array fabricated by bulk micromachining is found in U.S. patent application Ser. No. 09/353,554, filing date Jul. 14, 1999, as well as U.S. patent application Ser. No. 09/115,397, filing date Jul. 14, 1998.

In sacrificial micromachining, the substrate is left essentially untouched. Various thick layers of other materials are built up by vapor deposition, plasma-enhanced chemical vapor deposition (PECVD) or spin coating and selectively remain behind or are removed by subsequent processing steps. Thus, the resulting channel walls are chemically different from the bottom of the channels and the resist material remains as part of the microdevice. Typical resist materials for sacrificial micromachining are silicon nitride (Si3N4), polysilicon, thermally grown silicon oxide and organic resists such as SU-8 and polyimides allowing the formation of high aspect-ratio features with straight sidewalls.

A cross-sectional view of one example of a microchannel array that has been fabricated by sacrificial micromachining is found in U.S. patent application Ser. No. 09/353,554, filing date Jul. 14, 1999, as well as U.S. patent application Ser. No. 09/115,397, filing date Jul. 14, 1998.

In high-aspect ratio plating or LIGA, three-dimensional metal structures are made by high-energy X-ray radiation exposures on materials coated with X-ray resists. Subsequent electrodeposition and resist removal result in metal structures that can be used for precision plastic injection molding. These injection-molded plastic parts can be used either as the final microdevice or as lost molds. The LIGA process has been described by Becker et al., Microelectron Engineering (1986) 4:35-56 and Becker et al., Naturwissenschaften (1982) 69:520-523.

Alternative techniques for the fabrication of microchannel arrays include focused ion-beam (FIB) milling, electrostatic discharge machining (EDM), ultrasonic drilling, laser ablation (U.S. Pat. No. 5,571,410), mechanical milling and thermal molding techniques. One skilled in the art will recognize that many variations in microfabrication or micromachining techniques may be used to construct the device of the present invention.

Use of covers. In one embodiment, transparent or translucent covers are attached to the substrate via anodic bonding or adhesive coatings, resulting in microchannel arrays with inlet and outlet ports. In a preferred embodiment, the microchannel covers are glass, especially Pyrex or quartz glass. In alternative embodiments, a cover which is neither transparent nor translucent may be bonded or otherwise attached to the substrate to enclose the microchannels. In other embodiments the cover may be part of a detection system to monitor the interaction between biological moieties immobilized within the channel and an analyte. Alternatively, a polymeric cover may be attached to a polymeric substrate channel array by other means, such as by the application of heat with pressure or through solvent-based bonding.

Attachment of a cover to the microchannel array can precede formation of the organic thinfilm on the reactive sites. If this is the case, then the solution that contains the components of the organic thinfilm (typically an organic solvent) can be applied to the interior of the channels via microfabricated dispensing systems that have integrated microcapillaries and suitable entry/exit ports. Alternatively, the organic thinfilms can be deposited in the microchannels prior to enclosure of the microchannels. For these embodiments, organic thinfilms, such as monolayers, can optionally be transferred to the inner microchannel surfaces via simple immersion or through microcontact printing (see PCT Publication WO 96/29629). In one embodiment, the organic thinfilm in all of the microchannels is identical. In such a case, simple immersion of the microchannel array or incubation of all of the microchannel interiors with the same fluid containing the thinfilm components is sufficient.

Volume enclosed in each microchannel. The volume of each enclosed microchannel ranges from 5 nanoliters to 300 nanoliters. In one embodiment, the volume of an enclosed microchannel of the invention device is between 10 nanoliters and 50 nanoliters. Volumes of fluid may be moved through each microchannel by a number of standard means. In fact, simple liquid exchange techniques commonly used with capillary technologies can be used. For instance, fluid may be moved through the channel using standard pumps. Alternatively, more sophisticated methods of fluid movement through the microchannels such as electro-osmosis may be employed (for example, see U.S. Pat. No. 4,908,112).

Sample loading. In one embodiment, bulk-loading dispensing devices are used to load all microchannels of the device at once with the same fluid. Alternatively, integrated microcapillary dispensing devices may be microfabricated out of glass or other substrates to load fluids separately to each microchannel of the device. After formation of a microchannel, the sides, bottom, or cover of the microchannel or any portion or combination thereof, can then be further chemically modified to achieve the desired bioreactive and biocompatible properties.

Optional coating. The reactive sites of the device may optionally further comprise a coating between a substrate and its organic thinfilm. This coating may either be formed on the substrate or applied to the substrate. The substrate can be modified with a coating by using thin-film technology based, for example, on physical vapor deposition (PVD), thermal processing, or plasma-enhanced chemical vapor deposition (PECVD). Alternatively, plasma exposure can be used to directly activate or alter the substrate and create a coating. For instance, plasma etch procedures can be used to oxidize a polymeric surface (i.e., polystyrene or polyethylene to expose polar functionalities such as hydroxyls, carboxylic acids, aldehydes and the like).

The coating is optionally a metal film such as the metal films disclosed in section 6.3.1.2 above. In fact, the coating may be made in accordance with any of the coating embodiments described in section 6.3.1.2 above, including embodiments that have an adhesion layer or mediator between the coating and the substrate. Deposition or formation of the coating on the substrate (if such coatings are desired) occurs prior to the formation of organic thinfilms.

Description of organic thinfilm, coatings and substrate. The organic thinfilm on the reactive sites of the device forms a layer either on the substrate itself or on a coating covering the substrate. The organic thin films in accordance with this aspect of the invention include those disclosed in section 6.3.1.2. Additional disclosure on organic thin films in accordance with this aspect of the invention is found in U.S. patent application Ser. No. 09/353,554, filing date Jul. 14, 1999, which is a CIP of U.S. patent application Ser. No. 09/115,397, filing date Jul. 14, 1998. If the sites of the invention device comprise a self-assembled monolayer of molecules of the formula (X)ₐR(Y)_{b}, as defined in Section 6.3.1.2, then X, Y, a, b and R may be as defined in Section 6.3.1.2.

The devices in accordance with this aspect of the invention optionally further include a coating that is either formed on the substrate or is applied to the substrate. The materials used to form substrates and optional coatings in accordance with this aspect of the invention include those disclosed in section 6.3.1.2. Additional disclosure on substrate materials in accordance with this aspect of the invention is found in U.S. patent application Ser. No. 09/353,554, filing date Jul. 14, 1999.

Following formation of organic thinfilm on the reactive sites of the inventive device, the engineered proteins are immobilized on the monolayers. A solution containing the engineered protein to be immobilized can be exposed to the bioreactive, organic thinfilm covered sites of the microdevice by either dispensing the solution by means of microfabricated adapter systems with integrated microcapillaries and entry/exit ports. Such a dispensing mechanism would be suitable, for instance, if the reactive sites of the device were in covered, parallel microchannels. Alternatively, the engineered proteins transferred to uncovered sites of the device by using one of the arrayers based on capillary dispensing systems which are well known in the art and even commercially available. These dispensing systems are preferably automated. A description of an exemplary microarrayer comprising an automated capillary system can be found at http://cmgm.stanford.edu/pbrown/array.html and http://cmgm.stanford.edu/pbrown/mguide/index.html. The use of other printing techniques is also anticipated.

In an alternative embodiment of the invention, the reactive sites of the device are not contained within microchannels. For instance, the reactive sites of the inventive device may instead form an array of reactive sites like some of those described in U.S. Pat. No. 6,329,209 and “Arrays of Proteins and Methods of Use Thereof”, filed on Jul. 14, 1999, with the identifier 24406-0004 P1, for the inventors Peter Wagner, Dana Ault-Riche, Steffen Nock, and Christian Itin, both of which are herein incorporated by reference in their entirety.

Affinity tags and immobilization of the biological moieties. In some embodiments, the reactive sites of the device further comprise an affinity tag that enhances immobilization of the biological moiety onto the organic thinfilm. The affinity tags in accordance with this aspect of the invention include those disclosed in section 6.3.1.3 above. In an alternative embodiment of the invention, no affinity tag is used to immobilize the engineered protein onto the organic thinfilm. Rather, an amino acid in the engineered protein may be used to tether the protein to the reactive group of the organic thinfilm.

Adaptors. Another embodiment of the devices of the present invention comprises an adaptor that links the affinity tag to the immobilized biological moiety. In a preferred embodiment, the adaptor is a protein. In a preferred embodiment, the affinity tag, adaptor, and engineered protein together compose a fusion protein. Such a fusion protein is readily expressed using standard recombinant DNA technology. Adaptors that are proteins are especially useful to increase the solubility of the protein of interest and to increase the distance between the surface of the substrate or coating and the engineered protein. Use of an adaptor that is a protein can also be very useful in facilitating the preparative steps of protein purification by affinity binding prior to immobilization on the device. Examples of possible adaptors that are proteins include glutathione-S-transferase (GST), maltose-binding protein, chitin-binding protein, thioredoxin, green-fluorescent protein (GFP). GFP can also be used for quantification of surface binding.

Uses of the devices. Methods for using the devices of the present invention are provided by other aspects of the invention. The devices of the present invention are particularly well-suited for use in drug development, such as in high-throughput drug screening. Other uses include medical diagnostics and biosensors. The devices of the invention are also useful for functional proteomics. In each case, a plurality of engineered proteins can be screened for potential biological interactions in parallel.

In one aspect of the invention, a method for screening a plurality of different engineered proteins in parallel for their ability to interact with a component of a fluid sample is provided. This method comprises delivering the fluid sample to the reactive sites of one of the invention devices where each of the different engineered proteins is immobilized on a different site of the device, and then detecting for the interaction of the component with the immobilized biological moiety at each reactive site. In a preferred embodiment, each of the reactive sites is in a microchannel oriented parallel to microchannels of other reactive sites on the device and the microchannels are fabricated into the substrate.

6.6.4.3 Pillar Arrays

One embodiment of the invention is directed to chip such those disclosed in Indermuhle et al. PCT publication WO 01/62887 entitled “Chips Having Elevated Sample Surfaces”. The chip may comprise a base including a non-sample surface and at least one structure comprising a pillar. The at least one structure is typically in an array on the base of the chip. Each structure includes a sample surface that is elevated with respect to the non-sample surface of the chip. The sample surface of a structure may correspond to the top surface of the pillar. In other embodiments, the sample surface corresponds to an upper surface of a coating on the pillar.

Each sample surface may be adapted to receive a sample to be processed or analyzed while the sample is on the sample surface. The sample may include a component that is to be bound, adsorbed, absorbed, reacted, etc. on the sample surface. For example, the sample can be a liquid containing one or compounds and a liquid medium. Because a number of sample surfaces are on each chip, many samples may be processed or analyzed in parallel in embodiments of the invention.

The samples can be in the form of liquids when they contact the sample surfaces. When liquid samples are on the sample surfaces, the liquid samples may be in the form of discrete deposits. Any suitable volume of liquid may be deposited on the sample surfaces. For example, the liquid samples that are deposited on the sample surfaces may be on the order of 1 μL or less. In other embodiments, the liquid samples on the sample surfaces may be on the order of 10 nanoliters or less (e.g., 100 picoliters or less). In yet other embodiments, discrete deposits of liquids need not be left on the sample surfaces. For example, a liquid containing an engineered protein and a liquid medium may contact a sample surface. The engineered protein may bind to the sample surface and substantially all of the liquid medium may be removed from the sample surface, leaving only the engineered protein at the sample surface. Consequently, in some embodiments of the invention, liquid media need not be retained on the sample surfaces after liquid from a dispenser contacts the sample surface.

The liquid samples may be derived from biological fluids such as blood and rine. In some embodiments, the biological fluids may include organelles such as cells or molecules such as proteins and nucleic acid strands. When the chip is used to analyze, produce, or process a biological fluid, a biological molecule, or a compound in a solution, the chip may be referred to as a ‘biochip”.

The liquids provided by the dispenser comprise any suitable liquid media and any suitable components. Suitable components include analytes, engineered proteins (e.g., immobilized targets), and reactants. Suitable analytes or engineered proteins may be organic or inorganic in nature, and may be biological molecules, such as polypeptides, DNA, RNA, mRNA, antibodies, antigens, etc. Other suitable analytes may be chemical compounds that are potential candidate drugs. Reactants include reagents that can react with other components on the sample surfaces. Suitable reagents include biological or chemical entities that can process components at the sample surfaces.

The elevated sample surfaces upon which the samples are presented have specific properties. In some embodiments, the sample surfaces are rendered liquiphilic so that the sample surfaces are more likely to receive and retain liquid samples. For example, the sample surfaces may be hydrophilic. Alternatively or additionally, the sample surfaces have molecules that can bind, adsorb, absorb or react with components in the liquid samples deposited on the sample surfaces. In one example, the sample surface comprises one or more engineered proteins that react with an analyte in the liquid sample. In another example, the sample surface comprises a layer that is capable of receiving and binding the engineered proteins themselves. Accordingly, in embodiments of the invention, the nature of the sample surface changes as the sample structure changes.

Elevating the sample surfaces with respect to a non-sample surface provides a number of advantages. For example, by elevating the sample surfaces, potential liquid cross-contamination between the liquid samples on adjacent structures is minimized. A liquid sample on a sample surface does not easily flow to an adjacent sample surface, since the sample surfaces are separated by a depression. In some embodiments, cross-contamination between samples on adjacent sample surfaces is reduced even though rims are not present to confine a liquid sample to a sample surface. Since rims need not be present to confine the samples to their respective sample surfaces, the spacing between adjacent sample surfaces is reduced, thus increasing the density of the sample surfaces. As a result, more liquid samples are processed and/or analyzed per chip than in conventional methods. In addition, small liquid sample volumes can be used in embodiments of the invention so that the amount of reagents used is also decreased, thus resulting in lower costs.

In some embodiments, the side or portion of the side surfaces of the structures is provided with the same specific properties as the sample surface, or different selected properties from the sample surface. In one example, the side surfaces of a pillar of a chip is rendered hydrophobic while the sample surface of the pillar is hydrophilic. The hydrophilic sample surface of a pillar attracts the liquid samples, while the hydrophobic side surfaces of the pillar inhibit the liquid samples from flowing down the sides of the pillars. Accordingly, in some embodiments, a liquid sample is confined to the sample surface of a pillar without a well rim. Consequently, in embodiments of the invention, cross-contamination between adjacent sample surfaces may be minimized while increasing the density of the sample surfaces.

In an illustrative example of how a chip according to an embodiment of the invention can be used, a first dispenser deposits a number of liquid samples comprising respectively different proteins on the sample surfaces on a plurality of pillars on the base of the chip. The first dispenser uses a “passive valve” type dispenser. Passive valve type dispensers are described in further detail below. The different proteins, which may be engineered proteins, then bind to the different sample surfaces on respectively different pillars. A second dispenser, which may be the same or different than the first dispenser, then dispenses fluids comprising analytes or compounds onto the sample surfaces of the pillars. The fluids remain in contact with the sample surfaces for a predetermined period of time so that analytes in the fluids have time to interact (e.g., bind, react) with the proteins on the sample surfaces. The predetermined period of time may be greater than 30 seconds (e.g., greater than 1 minute). However, the time varies depending upon the particular interaction taking place. After the predetermined time has elapsed, the sample surfaces of the pillars are washed and/or exposed to wash or reagent liquids to remove any unbound analytes and/or compounds. The wash and/or reagent liquids can address each pillar independently or jointly, or by exposure to a liquid through, for example, flooding. The sample surfaces can then be analyzed to determine which, if any, of the analytes in the fluids may have interacted with the bound proteins.

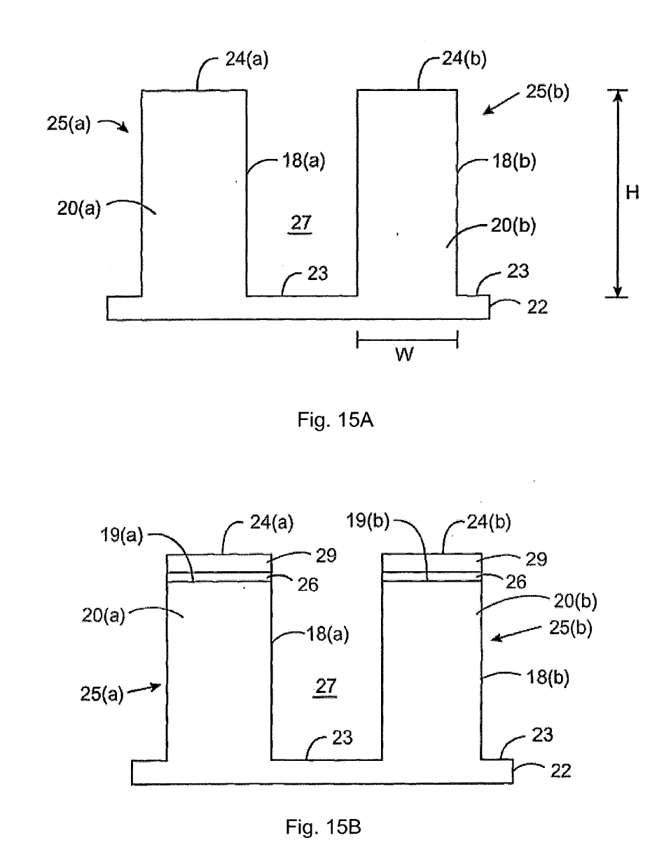

FIG. 15A shows a cross-sectional view of a chip according to an embodiment of the invention. The illustrated chip includes a base 22 and sample structures 25(a), 25(b) comprising pillars 20(a), 20(b). The base 22 and the pillars 20(a), 20(b) may form an integral structure formed from the same material. Alternatively, the base 22 and the pillars 20(a), 20(b) may be distinct and may be formed from different materials. Each pillar 20(a), 20(b) may consist of a single material (e.g., silicon), or may include two or more sections of different materials. The non-sample surface of the base 22 is typically planar. However, in some embodiments, base 22 has a non-planar surface. In one example, base 22 has one or more troughs. The structures containing the sample surfaces and the pillars may be in the trough. Any suitable material may be used in the base 22. Suitable materials include glass, silicon, or polymeric materials. Preferably, the base 22 comprises a machinable material such as silicon.

The pillars 20(a), 20(b) may be oriented substantially perpendicular with respect to the base 22. Each of the pillars 20(a), 20(b) includes a sample surface 24(a), 24(b) and side surfaces 18(a), 18(b). The side surfaces 18(a), 18(b) of the pillars 20(a), 20(b) can define respective sample surfaces 24(a), 24(b) of the pillars 20(a), 20(b). The sample surfaces 24(a), 24(b) may coincide with the top surfaces of the pillars 20(a), 20(b) and are elevated with respect to the non-sample surfaces 23 of the chip. The non-sample surfaces 23 and the sample surfaces 24(a), 24(b) may have the same or different coatings or properties. Adjacent sample surfaces 24(a), 24(b) are separated by a depression 27 that is formed by adjacent pillars 20(a), 20(b) and the non-sample surface 23. Pillars 20(a), 20(b) may have any suitable geometry. For example, the cross-sections (e.g., along a radius or width) of the pillars may be circular or polygonal. Each of the pillars 20(a), 20(b) may also be elongated. While the degree of elongation may vary, in some embodiments, the pillars 20(a), 20(b) have an aspect ratio of greater than 0.25 or more (e.g., 0.25 to 40). In other embodiments, the aspect ratio of the pillars is 1.0 or more. The aspect ratio may be defined as the ratio of the height H of each pillar to the smallest width W of the pillar. Preferably, the height of each pillar is greater than 1 micron. For example, the height of each pillar may range from 1 to 10 microns, or from 10 to 200 microns. Each pillar may have any suitable width including a width of less than 0.5 mm (e.g., 100 microns or less).

The liquids (not shown) can be in the form of discrete volumes of liquid and can be present on the sample surfaces 24(a), 24(b) of the pillars 20(a), 20(b), respectively. The liquid samples may be deposited on the sample surfaces 24(a), 24(b) in any suitable manner and with any suitable dispenser (not shown). The dispenser may include one or more passive valves within the fluid channels in the dispenser. Dispensers with passive valves are described in greater detail below.

The liquid samples may contain components (e.g., analytes, targets, engineered proteins) that are to be analyzed, reacted, or deposited on the sample surfaces 24(a), 24(b). Alternatively or additionally, the liquid samples may contain components that are to be deposited on the surfaces of the pillars 20(a), 20(b) for subsequent analysis, assaying, or processing. For example, the liquid samples on the pillars 20(a), 20(b) can comprise proteins. The proteins in the liquid samples may bind to the sample surfaces 24(a), 24(b). The proteins on the sample surfaces 24(a), 24(b) can then be analyzed, processed, and/or subsequently assayed, or used as engineered proteins for capturing analytes. For example, after binding proteins to the sample surfaces 24(a), 24(b), the bound proteins may be used as engineered proteins. Liquids containing analytes to be assayed against the engineered proteins may contact the surfaces 24(a), 24(b). The sample surfaces may then be analyzed to see if the analytes bind to the engineered proteins.

The liquid samples on the adjacent sample surfaces 24(a), 24(b) are separated from each other by the depression 27 between the adjacent structures. If, for example, a liquid sample flows off of the sample surface 24(a), the liquid sample flows into the depression 27 between the adjacent structures without contacting and contaminating the sample on the adjacent sample surface 24(b). To help retain the samples on the sample surfaces 24(a), 24(b), the side surfaces 18(a), 18(b) of the pillars 20(a), 20(b) may be rendered liquiphobic or may be inherently liquiphobic. For example, the side surfaces 18(a), 18(b) may be coated with a hydrophobic material or may be inherently hydrophobic. In other embodiments, the side surfaces 18(a), 18(b) of the pillars may also be coated with a material (e.g., alkane thiols or polyethylene glycol) resistant to analyte binding. The non-sample surface 23 may also be resistant to analyte binding or may be liquiphobic, or may consist partially or fully of the same material as the sample surfaces 24(a), 24(b).

In some embodiments, the pillars have one or more channels that surround, wholly or in part, one or more pillars on the base. Examples of such channels are discussed in U.S. patent application Ser. No. 09/353,554. This U.S. patent application also discusses surface treatment processes and compound display processes that can be used in embodiments of the invention. The top regions of the sample structures 25(a), 25(b) may include one or more layers of material. For example, FIG. 15B shows a cross-sectional view of a chip with pillars 20(a), 20(b) having a first layer 26 and a second layer 29 on the top surfaces 19(a), 19(b) of the pillars 20(a), 20(b). In this example, the sample surfaces 24(a), 24(b) of the structures 25(a), 25(b) may correspond to the upper surface of the second layer 29. In some embodiments, the top regions of the structures 25(a), 25(b) may be inherently hydrophilic or rendered hydrophilic. As explained in further detail below, hydrophilic surfaces are less likely to adversely affect proteins that may be at the top regions of the structures 25(a), 25(b).

The first and the second layers 26,29 may comprise any suitable material having any suitable thickness. The first and the second layers 26,29 can comprise inorganic materials and may comprise at least one of a metal or an oxide such as a metal oxide. The selection of the material used in, for example, the second layer 29 (or for any other layer or at the top of the pillar) may depend on the molecules that are to be bound to the second layer 29. For example, metals such as platinum, gold, and silver may be suitable for use with linking agents such as sulfur containing linking agents (e.g., alkanethiols or disulfide linking agents), while oxides such as silicon oxide or titanium oxide are suitable for use with linking agents such as silane-based linking agents. The linking agents can be used to couple entities such as engineered proteins to the pillars.

In one example, the first layer 26 comprises an adhesion metal such as titanium and is less than 5 nanometers thick. The second layer 29 may comprise a noble metal such as gold and may be 100 to 200 nanometers thick. In another embodiment, the first layer 26 may comprise an oxide such as silicon oxide or titanium oxide, while the second layer 29 may comprise a metal (e.g., noble metals) such as gold or silver. Although the example shown in FIG. 15B shows two layers of material on the top surfaces 19(a), 19(b) of the pillars 20(a), 20(b), the top surfaces 19(a), 19(b) may have more or less then two layers (e.g., one layer) on them. Moreover, although the first and the second layers 26,29 are described as having specific materials, it is understood that the first and the second layers 26,29 may have any suitable combination of materials.

The layers on the pillars may be deposited using any suitable process. For example, the previously described layers may be deposited using processes such as electron beam or thermal beam evaporation, chemical vapor deposition, sputtering, or any other technique known in the art.

In embodiments of the invention, an affinity structure may be on a pillar, alone or in combination with other layers. For example, the affinity structure may be on an oxide or metal layer on a pillar or may be on a pillar without an intervening layer. Preferably, the affinity structure comprises organic materials. In some embodiments, the affinity structure may consist of a single layer comprising molecules that are capable of binding to specific analytes (e.g., proteins). For instance, the affinity structure may comprise a single layer of engineered proteins that are bound to the surface of, for example, a metal or oxide layer on a pillar. The engineered proteins can bind to components in a liquid medium through a covalent or a non-covalent mechanism. The affinity structure (and the elements of the affinity structure) can be used to increase the spacing between a top surface (e.g., a silicon surface) of a pillar and a protein that is attached to the top surface of the pillar. The spacing can decrease the likelihood that the attached protein might become deactivated by, for example, contacting a solid surface of the sample structure.

In other embodiments, the affinity structure may comprise an organic thin film, affinity tags, adaptor molecules, and engineered proteins, alone or in any suitable combination. When any of these are used together, the organic thin film, affinity tags, adaptor molecules, and the engineered proteins may be present in two or more sublayers in the affinity structure. For example, the affinity structure may include three sublayers, each sublayer respectively comprising an organic thin film, affinity tags, and adaptor molecules.

The organic thin film, affinity tags, and adaptor molecules may have any suitable characteristics. An “organic thin film” is a normally a thin layer of organic molecules that is typically less than 20 nanometers thick. Preferably, the organic thin film is in the form of a monolayer. A “monolayer” is a layer of molecules that is one molecule thick. In some embodiments, the molecules in the monolayer are oriented perpendicular, or at an angle with respect to the surface to which the molecules are bound.

The monolayer may resemble a “carpet” of molecules. The molecules in the monolayer may be relatively densely packed so that proteins that are above the monolayer do not contact thelayer underneath the monolayer. Packing the molecules together in a monolayer decreases the likelihood that proteins above the monolayer will pass through the monolayer and contact a solid surface of the sample structure. An “affinity tag” is a functional moiety capable of directly or indirectly immobilizing a component such as a protein. The affinity tag may include a polypeptide that has a functional group that reacts with another functional group on a molecule in the organic thin film. Suitable affinity tags include avidin and streptavidin. An “adaptor” may be an entity that directly or indirectly links an affinity tag to a pillar. In some embodiments, an adaptor may provide an indirect or direct link between an affinity tag and an engineered protein. Alternatively or additionally, the adaptor may provide an indirect or direct link between the pillar and, an affinity tag or a engineered proteins. Examples of organic thin films, affinity tags, and adaptors are described sections 6.3.1 and 6.3.2 above and in U.S. patent application Ser. Nos. 09/115,455, 09/353,215, and 09/353,555. These U.S. patent applications describe various layered structures that can be on the pillars in embodiments of the invention.

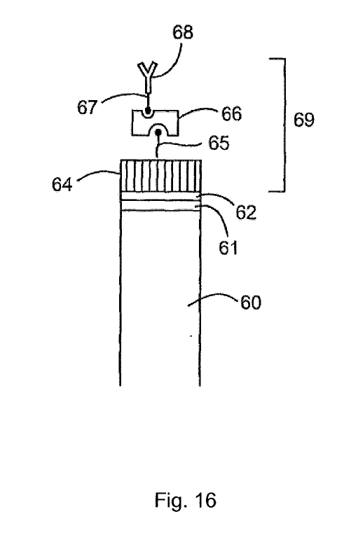

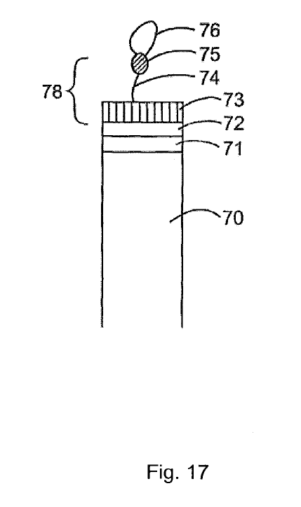

The materials of the sublayers may be bound to the other sublayer materials, the pillars, or layers on the pillars by a covalent or a non-covalent bonding mechanism. Examples of chip structures having affinity structures on the pillars are shown in FIGS. 16 and 17. FIG. 16 shows a cross-sectional view of a sample structure having an elevated sample surface. The sample structure includes a pillar 60. An interlayer 61 including an oxide such as silicon oxide is at the top surface of the pillar 60. The interlayer 61 may be used to bind the coating layer 62 to the pillar 60. The coating layer 62 may include another oxide such as titanium oxide. An affinity structure 69 is on the coating layer 62. The affinity structure 69 may include a monolayer 64 with organic molecules such as polylysine or polyethylene glycol. In some embodiments, the molecules in the monolayer 64 are linear molecules that are oriented generally perpendicular to, or at an angle with, the surface the coating layer 62. Each of the organic molecules in the monolayer 64 may have functional groups at both ends to allow the ends of the molecules to bind to other molecules.

A set of molecules including a first adaptor molecule 65 such as biotin, an affinity tag 66 such as avidin or streptavidan, a second adaptor molecule 67 such as biotin, and a engineered protein 68 are linked together. The set of molecules is bound to the monolayer 64. In this example, the engineered protein 68 is adapted to receive and capture an analyte or compound in a liquid sample that is on the pillar 60. The compound may be, for example, a low molecular weight compound as described in Section 5.1. For simplicity of illustration, only one set of molecules is shown in FIG. 16. However, it is understood that in embodiments of the invention, many such sets of molecules may be present on the monolayer 64.

The embodiment shown in FIG. 16 has an affinity structure that has a number of sublayers. The affinity structures used in other embodiments of the invention may include more or less sublayers. For example, FIG. 16 shows a cross-sectional view of another sample structure having an affinity structure with fewer sublayers. The structure shown in FIG. 17 includes a pillar 70. An interlayer 71 including a material, such as silicon dioxide, is at the top surface of the pillar 70. A coating layer 72 including, for example, a metal oxide (e.g., titanium oxide) may be on the interlayer 71. An affinity structure 78 may be on the coating layer 72. The affinity structure 78 may include a monolayer 73, an affinity tag 74, and an adaptor molecule 75. The affinity tag 74 may be on the monolayer 73 and may couple the adaptor molecule 75 to the monolayer 73. The adaptor molecule 75 may, in turn, bind an engineered protein 76 to the affinity tag 74. The affinity structure components separate the sample surface from the top surface of the pillar. As noted above, proteins may deactivate when they come into contact with certain solid surfaces. The affinity structure serves as a barrier between the pillar and any components in a liquid sample that are to be captured. This reduces the possibility that the top surface, of the pillar will deactivate proteins in a liquid sample on the pillar. As shown in FIGS. 16 and 17, for example, the bound engineered protein 76 and the bound engineered protein 68 are not in likely to contact a solid surface (e.g., the solid surfaces of the coating layers 62, 72). Consequently, the presence of the affinity structure 69,78 decreases the likelihood that contact sensitive molecules such as proteins will be adversely affected by contact with a solid surface. To further reduce this possibility, the materials of the affinity structure may contain materials that are less likely to inactivate proteins.

In some embodiments, the pillars are present in an array on a base of the chip. The pillar array is either regular or irregular. In one example, the array has even rows of pillars forming a regular array of pillars. The density of the pillars in the array may vary. In one example, the density of the pillars is 25 pillars per square centimeter or greater (e.g., 10,000 or 100,000 per cm² or greater). Although the chips have any suitable number of pillars, in some embodiments, the number of pillars per chip is greater than 10, 100, or 1000. The pillar pitch (i.e., the center-to-center distance between adjacent pillars) it typically 500 microns or less (e.g., 150 microns).

In some embodiments, each pillar includes a porous material such as a hydrogel material. In embodiments of the invention, all, part, or parts of the pillar have the same or different degree of porosity. For instance, different strata within a pillar may be porous and can have different properties. By using a porous material, liquid samples can pass into the porous material and the pillar can hold more liquid sample than would be possible if the pillar was non-porous. Consequently, more liquid sample can be present in a porous pillar than a pillar having similar cross-sectional dimensions. If the liquid sample contains a fluorescent material, for example, more fluorescent material is retained by the pillar than by a non-porous pillar. A higher quality signal (e.g., a stronger signal) is produced as a result of the increased amount of fluorescent material in the porous pillar as compared with a non-porous pillar that only has fluorescent material on the top surface of the pillar.

In some embodiments, fluid passages are provided in the pillars of the chip. In one embodiment, a fluid passage extends through both the base and the pillars. A fluid such as a gas passes through the fluid passages toward the sample surfaces on the pillars to remove substances from the sample surfaces. A cover chip with corresponding apertures is placed over the fluid passages in the pillar so that the apertures are over the sample surfaces. Gas flows through the fluid passages to carry processed samples on the upper surfaces of the pillars to an analytical device such as a mass spectrometer. In a typical process of using the assembly, liquids from a dispenser (not shown) contact the sample surfaces on the pillars of a sample chip. The liquids process substances on the sample surfaces on the pillars. In one example, the liquids comprise reagents that process proteins on the sample surfaces. After processing, the chip is separated from the dispenser, and the cover chip is placed on the sample chip with the pillars. The apertures of the cover chip are respectively over the sample surfaces, and gas flows through fluid passages that extend through the pillars. The gas removes the processed substances from the sample surfaces and carries the processed substances through the apertures in the cover chip and to an analysis device, such as a mass spectrometer. Chips with fluid passages may also be used to pass liquids upward through the fluid passages in order to deposit the liquid on the sample surfaces of the sample chip (i.e., on the pillars). In yet other embodiments, the fluid passages are used to keep components at the sample surfaces hydrated. Hydrating gases or liquids (e.g., water) can pass through the fluid passages to keep any components on the sample surfaces hydrated. Often, the act of keeping proteins on the sample surfaces hydrated makes them less likely to denature. In some embodiments, the fluid passages are coupled to a sub-strata porous region of the pillar. This serves to act as a liquid reservoir in order to supply liquid to the sample surface.

Pillar fabrication. The chip pillars are fabricated in any suitable manner and using any suitable material. In some embodiments, an embossing, etching and/or a molding process is used to form the pillars on the base of the chip. In one example, a silicon substrate is patterned with photoresist where the top surfaces of the pillars are formed. An etching process, such as a deep reactive ion etch, is then performed to etch deep profiles in the silicon substrate and to form a plurality of pillars. Side profiles of the pillars are modified by adjusting process parameters such as the ion energy used in a reactive ion etch process. If desired, the side surfaces of the formed pillars are coated with material such as a hydrophobic material while the top surfaces of the pillars are covered with photoresist. After coating, the photoresist is removed from the top surfaces of the pillars. Processes for fabricating pillars are well known in the semiconductor industry.

Assemblies. Other embodiments of the invention are directed to fluid assemblies. The fluid assemblies according to embodiments of the invention include a sample chip and a dispenser that dispenses one or more fluids on the sample surfaces of the chip. In some embodiments, a plurality of liquids is supplied to the fluid channels in a dispenser. The liquids supplied to the different fluid channels are the same or different and contain the same or different components. In one example, each of the liquids in respective fluid channels includes different analytes to be assayed. In another example, the liquids in respective fluid channels contain different engineered proteins to be coupled to the pillars of the sample chip. The dispenser may provide liquids to the sample surfaces in parallel.

The chips used in the assemblies may be the same as the previously described chips. For example, the chips in the assemblies may include structures having elevated sample surfaces and pillars. The dispenser has any suitable characteristics, and can be positioned above the sample chip when liquids are dispensed onto the sample chip. Pressure may be applied to the liquids to dispense the liquids. The dispenser may include passive or active valves to control liquid flow.

Active liquid valves are well known in the art. These valves control the flow or location of a liquid by actively changing a physical parameter. Some examples follow: 1) heat or light change the liquiphilic properties of a polymer that is used to control the location of a liquid; 2) electric potential that is used to induce an electrokinetic flow; 3) microelectromechanical structures used to block or unblock a liquid channel; and 4) the movement of magnetic particles or features in a channel to influence the liquid behavior. In some embodiments, the dispensers have at least one passive valve per fluid channel. Preferably, the dispenser includes a plurality of nozzles. The plurality of nozzles is capable of providing different liquids containing different components to different sample surfaces of the pillars substantially simultaneously. In one instance, an array of one hundred sample surfaces on a chip is matched with a dispenser having one hundred sample nozzles that are arranged in a pattern similar to the array of sample surfaces. In other embodiments, the dispenser has one or more nozzles that provide liquids on different sample surfaces in series. Examples of dispensers that are used in embodiments of the invention include ring-pin dispensers, micropipettes, capillary dispensers, ink-jet dispensers, hydrogel stampers, and dispensers comprising passive valves. In some embodiments, the dispensers are in the form of a chip with a plurality of fluid channels. In these embodiments, each of the fluid channels have an end that terminates at a bottom face of the dispenser chip.

The dimensions of the fluid channels in the dispenser vary. In one example, a cross-sectional dimension of a fluid channel in the dispenser is between 1.0 micron to 500 microns (e.g., 1.0 micron to 100 microns). The dispensers used in embodiments of the invention are made using any suitable process known in the art. The dispenser is made, for example, by a 3-D stereo lithography, mechanical drilling, ion etching, or a reactive ion etching process. In some assembly embodiments, the sample structures of the chip is cooperatively structured to fit into fluid channels in a dispenser. The sample structures and their corresponding sample surfaces may be aligned with the fluid channels. After aligning, the sample surfaces may be positioned in the fluid channels or at the ends of the fluid channels. Fluids in the fluid channels then contact the sample surfaces of the structures. In some embodiments, pressure (e.g., caused by pneumatic forces, electrophoretic or electrowetting forces) is applied to a liquid in a fluid channel so that the liquid flows and contacts the sample surface in the fluid channel. In other embodiments, the distance between the sample surface and the liquid in a fluid channel decreases until they contact each other. The chip and/or the dispenser may move toward each other to decrease the spacing between the sample surface and the liquid in the fluid channel. The fluid channels in the dispenser may serve as reaction chambers (or interaction chambers) that can house respectively different interactions such as reactions or binding events. Each sample surface and the walls of a corresponding fluid channel may form a reaction chamber. In a typical assembly, each individual reaction chamber houses a different event (e.g., a different reaction or binding event).

Illustratively, a dispenser provides liquids to the sample surfaces of the chip structures. The liquids contain molecules that may or may not interact with engineered proteins bound to the chip sample surfaces. First, the sample structures containing the sample surfaces are aligned with the fluid channels. After aligning, the sample surfaces are inserted into or positioned proximate to the fluid channels. While the sample surfaces are in or proximate to the fluid channels, the liquids in the fluid channels of the dispenser flow and contact the sample surfaces. This allows the engineered protein bound to the sample surfaces and the molecules in the liquids to react or interact with each other in a nearly closed environment. The interactions or reactions can take place minimizing the exposure of the liquid samples on the sample surfaces to a gaseous environment such as air. This reduces the likelihood that the liquid samples will evaporate. After a predetermined time has elapsed, the sample surfaces are withdrawn from the fluid channels, and/or the chip and the dispenser are separated from each other. The sample surfaces of the chip are then rinsed. Products of the reactions or interactions remain on the sample surfaces. The products at the sample surfaces are then be analyzed to determine, for example, if a binding reaction has taken place.

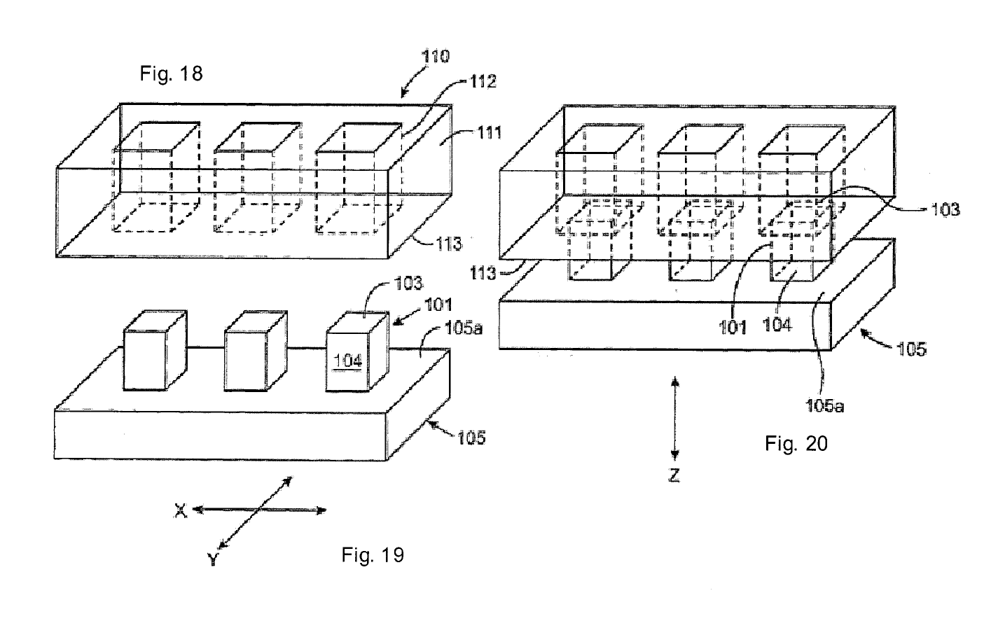

Some assembly embodiments are described with reference to FIGS. 18 to 20. FIG. 18 shows a dispenser 110 and FIG. 19 shows a chip 105. The chip 105 includes a plurality of pillars 101 on a base 105. Each pillar 101 has a top sample surface 103 and a side surface 104. The sample surface 103 is elevated with respect to a non-sample surface of the base 105a.

Dispenser 110 includes a body 111 having at least one fluid channel 112 defined in the body 111. In this example, the fluid channels 112 are substantially vertical. As noted above, the fluid channels 112 define reaction chambers that house chemical or biological reactions or interactions. At least a portion of the fluid channels 112 is oriented in a z direction with respect to an x-y plane formed by the body 111 of the dispenser 110. In this example, the fluid channels 112 illustrated in FIG. 18 are vertical and have one end terminating at an upper surface of the body 111 and the other end terminating at a lower surface of the body 111. In other dispenser embodiments, the fluid channels 112 have horizontal and vertical portions. For example, one end of a fluid channel originates at an upper surface of the body and passes horizontally across the upper surface of the body. At some predetermined point on the body, the orientation of the fluid channel changes from a horizontal orientation to a vertical orientation and terminates at a lower surface of the body of the dispenser. Moreover, although the number of fluid channels 112 in the dispenser is shown to be equal to the number of pillars 101 in the assembly shown in FIGS. 18 and 19, the number of fluid channels and the number of pillars of a chip may be different in other embodiments.

In some embodiments, the walls defining the fluid channels 112, as well as a bottom surface 113 of the dispenser 110 are coated with various materials that influence the behavior of the liquid in the fluid channels 112 (e.g., wetting). For instance, the fluid channel walls may be coated with materials that increase or decrease the interaction between fluid channel walls and the liquids in the fluid channels. In one example, the walls defining the fluid channels 112 are coated with a hydrophilic material. Proteins, for example, are less likely to denature if they come in contact with a hydrophilic surface than with a non-hydrophilic surface.

The fluid channels 112 in the dispenser 110 may be cooperatively structured to receive the pillars 101. For example, as shown in FIG. 19, the pillars 101 of the chip 105 may insert into the fluid channels 112 in the body of the dispenser 110. In this regard, the axial cross-sectional area of each of the fluid channels 112 in the dispenser 110 may be greater than the axial cross-sectional area of the pillars 101. When the pillars 101 are inserted into the fluid channels 112 in the dispenser 110, the sample surfaces 103 of the pillars 101 may be within respective fluid channels 112.

The chip 105 and the dispenser 110 may each have one or more alignment members so that they can be aligned with each other and the pillars can be aligned with the fluid channels. In one embodiment, the alignment members are alignment marks or alignment structures. Typical alignment structures are, for example, a pin and a corresponding hole. For instance, the edges of the chip 105 may have one or more pins (not shown) that are longer than the pillars 101. These pins may be inserted into corresponding holes (not shown) at the edges of the dispenser 110 to align the chip 105 and the dispenser 110 and consequently align the pillars 101 with the fluid channels 112. The alignment members may be optical, mechanical, or magnetic. For example, in some embodiments, the alignment members may be high aspect ratio linear channels which permit light passage when, for example, the chip and the dispenser are operatively aligned. Alternatively, a magnetic region may induce a signal in a detector once, for example, the chip and the dispenser are operatively aligned.

The assembly embodiments may be used to perform assays. Illustratively, biological molecules such as proteins are bound to the top surfaces 103 of the pillars 101. The pillars 101 are then aligned with the fluid channels 112 of the dispenser 110 and liquids containing different potential candidate drugs pass through the different vertical fluid channels 112 and to the sample surfaces of the pillars 101. Potential interactions or reactions between the different candidate drugs and the proteins take place within these reaction chambers formed by the pillars 101 and the fluid chambers 112. A predetermined amount of time is permitted to elapse to allow any reactions or interactions to occur. In some embodiments, the time is 1 minute or more. In other embodiments, the elapsed time surpasses 30 minutes. After any reactions or interactions occur, chip 105 and dispenser 110 are separated from each other. Discrete liquid samples may be present on the top surfaces 103 of the chip 105 after the chip 105 is separated from the dispenser 110. Then, the sample surfaces 103 of the pillars 101 are washed. The sample surfaces 103 are then be analyzed to determine which, if any, of the potential candidate drugs bind to the engineered proteins on the top surfaces 103 of the pillars 101. To help identify the candidate drugs, the candidate drugs may have different fluorescent tags bound to them prior to being on the sample surfaces 103.

In another embodiment, the fluid channels 112 have liquids with engineered proteins that are to be bound to the top surfaces of the pillars 101. The pillars 101 are introduced in the fluid channels 112, thereby forming a small reaction chamber together with the inner fluid channel walls, the molecules in the liquid are thereby given the opportunity to react or bind (e.g., without leaving a distinct deposit of liquid on the pillar). Alternatively, the liquids are deposited on the pillars 101 and the engineered proteins bind to the top surfaces 103 of the pillars 101. The dispenser 110 and the chip 105 are separated and the engineered proteins bound to the top surfaces are used to capture analytes and/or compounds for analysis.

The assemblies may include one or more passive valves. A passive valve stops the flow of liquid inside or at the end of a capillary using a capillary pressure barrier that develops when the characteristics of the capillary or mini channel changes, such as when the capillary or channel cross-section changes abruptly, or when the materials of structures defining the fluid channels change abruptly. Passive valves are discussed in P. F. Man et al., “Microfabricated Capillary-Driven Stop Valve and Sample Injector,” IEEE 11^{th} Annual Int. MEMS Workshop, Santa Clara, Calif., September 1999, pp. 45-50, and M. R. McNeely et al., “Hydrophobic Microfluidics,” SPIE Conf. on Microfluidic Devices and Systems II, Santa Clara, Calif., September 1999, vol. 3877, pp. 210-220. Passive valves are unlike active valves that completely close off a fluid channel with a physical obstruction.

In an illustrative example of how an assembly with a passive valve is used, the structures of a chip are inserted into respective fluid channels in a dispenser. Each fluid channel has one, two, or three or more passive valves. For instance, each fluid channel has a passive valve that is formed by an abrupt structural change in the geometry of a fluid channel. In one example, the walls of a fluid channel form a step structure. When a liquid encounters the step structure at a predetermined pressure, the liquid stops flowing.

Passive valves can also be formed when the structures containing the sample surfaces are within or are positioned at the ends of the fluid channels. For example, a pillar may be inserted into a fluid channel so that there is a space between the side surfaces of the pillar that is in the fluid channel and the fluid channel walls around the pillar. The portion of the fluid channel where the pillar resides may have an annular configuration. As liquid flows towards the pillar, the geometry of the fluid channel changes from a cylindrical configuration to an annular configuration. At a predetermined pressure, the liquid stops flowing at this geometry change. Additional pressure is needed to cause the liquid to flow past this geometry change. Different pressures may be applied to initiate the flow of liquid past each of the passive valves in the fluid channel. For example, two different levels of pressure may be applied to a fluid in a fluid channel to move a liquid past two different passive valves.

In one specific example of an assembly with a dispenser using one or more passive valves, a chip including pillars is used with a dispenser containing a plurality of fluid channels. The pillars are inserted into the fluid channels and the chip is brought into contact with the dispenser. Before or after insertion, a first pressure is applied to the liquids in the fluid channels to push the fluid samples to, but not substantially past, the first passive valve. A second pressure is then applied to the fluid samples to push the samples past the first passive valve so that the liquids are in contact with the pillars. The samples do not pass the second passive valve, which is defined by the pillar and the channel walls. After the liquids in the fluid channels contact the sample surfaces, the pressure applied to the liquids is decreased. Then, the dispenser and the chip are separated from each other to separate the sample surfaces from the bulk of the liquids in the fluid channels. In this step, the pillars are withdrawn from the fluid channels and liquid samples remains on the sample surfaces. After liquid samples are transferred to the sample surfaces, processes such as evaporation and the formation of an air-liquid interface will have little or no adverse effect on the deposited components in the liquid samples. Any residual solvent or material on the sample surface is rinsed away leaving the desired components on the sample surfaces.

In other embodiments, the structures are inserted into the fluid channels until contact is made with liquids within respective channels. In these embodiments, added pressure is not be applied to the fluids in the fluid channels to bring the fluids in contact with the sample surfaces of the structures.

The dispensers according to embodiments of the invention have a number of advantages. For instance, unlike conventional ring-pin dispensers, embodiments of the invention can deliver a large number of liquids to the sample surfaces in parallel. In some embodiments of the invention, 10,000 or more fluid channels are used to dispense 10,000 liquid samples. In comparison, conventional ring-pin dispensers have only 30 ring pins per assembly. Also, unlike a capillary pin dispenser that can potentially touch a sample surface thus damaging the dispenser and the sample surface, many of the described dispenser embodiments do not come in contact with the sample surface. Moreover, unlike many conventional dispensers, the assembly embodiments of the invention reduce the likelihood of forming an air-liquid interface, since droplets are not formed when liquid is transferred from a dispenser to a chip. As the volume of a drop gets smaller, the surface to volume ratio of the drop gets larger leading to problematic interactions between the molecules in the liquid that are to be transferred to the sample surface and the air-liquid interface of the drop. In some embodiments of the invention, droplets of liquid are not formed, thus minimizing the formation of a liquid sample with a gas/liquid interface with a high surface to volume ratio.

### 6.7 NUCLEIC ACIDS OF THE PRESENT INVENTION

The engineered proteins of the present invention may further be defined by the nucleic acid that codes for the engineered proteins. Accordingly, one embodiment of the present invention provides the nucleic acid that codes for novel engineered protein. The primary sequence of at least one portion of the novel engineered protein is determined by an engineering scheme, such as the randomization scheme disclosed in the experimental section below. In some embodiments, the corresponding parent protein of this novel engineered protein comprises a three-layer swiveling β/β/α domain in which the central beta sheet is parallel and the other beta sheet is antiparallel. In some embodiments, the corresponding parent protein is rubredoxin. In one embodiment, this nucleic acid is DNA or RNA. In another embodiment, the engineered protein is characterized by its ability to bind to a compound that does not specifically bind to the corresponding parent protein.

6.7.1 High Stringency

One embodiment of the present invention provides a nucleic acid that hybridizes under conditions of high stringency to nucleotide 760 through nucleotide 1215 of SEQ ID NO: 2. Nucleotide 760 through nucleotide 1215 of SEQ ID NO: 2 (FIG. 3B) codes for the substrate-binding domain of the Thermoplasma acidophilum thermosome (residue 214 through residue 365 of SEQ ID NO: 1). Another embodiment of the present invention provides a nucleic acid that hybridizes under conditions of high stringency to a polynucleotide that is complementary to nucleotides 760 through 1215 of SEQ ID NO: 2. Another embodiment of the present invention provides a nucleic acid that hybridizes under conditions of high stringency to the nucleotide sequence of SEQ ID NO: 34 (FIG. 24) or the complement of SEQ ID NO: 34 (not shown).

High stringency conditions are known in the art; see for example Maniatis et al., Molecular Cloning: A Laboratory Manual, 2d Edition, 1989, and Short Protocols in Molecular Biology, ed. Ausubel et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be 5-10° C. lower than the thermal melting point (Tm) for the specific sequence at a defined ionic strength pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than 1.0 M sodium ion, typically 0.01 to 1.0 M sodium ion concentration (or other salts) at pH 7.0 to 8.3 and the temperature is at least 30° C. for short probes (e.g. 10 to 50 nucleotides) and at least 60° C. for long probes (e.g. greater than 50 nucleotides). Stringent conditions may also be achieved with the addition of destabilizing agents such as formamide.

By way of example and not limitation, procedures using conditions of high stringency for regions of hybridization of over 90 nucleotides are as follows. Prehybridization of filters containing DNA is carried out for 8 h to overnight at 65° C. in buffer composed of 6×SSC, 50 mM Tris-HCl (pH 7.5), 1 mM EDTA, 0.02% PVP, 0.02% Ficoll, 0.02% BSA, and 500 μg/ml denatured salmon sperm DNA. Filters are hybridized for 48 h at 65° C. in prehybridization mixture containing 100 μg/ml denatured salmon sperm DNA and 5-20×10⁶ cpm of ³²P-labeled probe. Washing of filters is done at 37° C. for 1 h in a solution containing 2×SSC, 0.01% PVP, 0.01% Ficoll, and 0.01% BSA. This is followed by a wash in 0.1×SSC at 50° C. for 45 min before autoradiography. Other conditions of high stringency that may be used depend on the nature of the nucleic acid (e.g. length, GC content, etc.) and the purpose of the hybridization (detection, amplification, etc.) and are well known in the art. For example, stringent hybridization of an oligonucleotide of approximately 15-40 bases to a complementary sequence in the polymerase chain reaction (PCR) is done under the following conditions: a salt concentration of 50 mM KCl, a buffer concentration of 10 mM Tris-HCl, a Mg²⁺ concentration of 1.5 mM, a pH of 7-7.5 and an annealing temperature of 55-60° C. The skilled artisan will recognize that the temperature, salt concentration, and chaotrope composition of hybridization and wash solutions may be adjusted as necessary according to factors such as the length and nucleotide base composition of the probe.

6.7.2 Moderate Stringency

Another embodiment of the present invention provides a nucleic acid that hybridizes under conditions of moderate stringency to nucleotide 760 through nucleotide 1215 of SEQ ID NO: 2. Still another embodiment of the present invention provides a nucleic acid that hybridizes under conditions of moderate stringency to a polynucleotide that is complementary to nucleotides 760 through 1215 of SEQ ID NO: 2. Another embodiment of the present invention provides a nucleic acid that hybridizes under conditions of moderate stringency to the nucleotide sequence of SEQ ID NO: 34 (FIG. 24) or the complement of SEQ ID NO: 34 (not shown). As used herein, conditions of moderate stringency, as known to those having ordinary skill in the art, and as defined by Sambrook et al., Molecular Cloning: A Laboratory Manual, 2nd Ed. Vol. 1, pp. 1.101-104, Cold Spring Harbor Laboratory Press, 1989), include use of a prewashing solution for the nitrocellulose filters 5×SSC, 0.5% SDS, 1.0 mM EDTA (pH 8.0), hybridization conditions of 50% formamide, 6×SSC at 42° C. (or other similar hybridization solution, or Stark's solution, in 50% formamide at 42° C.), and washing conditions of 60° C., 0.5×SSC, 0.1% SDS. See also, Ausubel et al., eds., in the Current Protocols in Molecular Biology series of laboratory technique manuals, © 1987-1997, Current Protocols, © 1994-1997, John Wiley and Sons, Inc.). The skilled artisan will recognize that the temperature, salt concentration, and chaotrope composition of hybridization and wash solutions may be adjusted as necessary according to factors such as the length and nucleotide base composition of the probe.

6.7.3 Low Stringency

Yet another embodiment of the present invention provides a nucleic acid that hybridizes under conditions of low stringency to nucleotide 760 through nucleotide 1215 of SEQ ID NO: 2. Nucleotide 760 through nucleotide 1215 of SEQ ID NO: 2 codes for the substrate-binding domain of the Thermoplasma acidophilum thermosome (residue 214 through residue 365 of SEQ ID NO: 1). Another embodiment of the present invention provides a nucleic acid that hybridizes under conditions of low stringency to a polynucleotide that is complementary to nucleotides 760 through 1215 of SEQ ID NO: 2. Another embodiment of the present invention provides a nucleic acid that hybridizes under conditions of moderate stringency to the nucleotide sequence of SEQ ID NO: 34 (FIG. 24) or the complement of SEQ ID NO: 34 (not shown). By way of example and not limitation, procedures using conditions of low stringency for regions of hybridization of over 90 nucleotides are as follows (see also Shilo and Weinberg, 1981, Proc. Natl. Acad. Sci. U.S.A. 78, 6789-6792). Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and re-exposed to film. Other conditions of low stringency that may be used are well known in the art (e.g., as employed for cross-species hybridizations).

6.7.4 Expectation Values

Still another embodiment of the present invention provides a nucleic acid in which the overall sequence similarity of the nucleic acid to nucleotides 760 through 1215 of SEQ ID NO:2 is characterized by an expectation value that is selected from a range of 1e-4 to 1e-9. Yet another embodiment of the present invention provides a nucleic acid in which the overall sequence similarity of the nucleic acid to nucleotides 760 through 1215 of SEQ ID NO:2 is characterized by an expectation value that is selected from a range of 1e-4 to 1e-6. An expectation value is a measure of the likelihood that an alignment between two sequences might occur by chance. The expectation value range 1e-4 to 1e-9 includes any alignment between a target and query sequence in which the likelihood that such an alignment would occur by chance is in the range from 1 in 10,000 to 1 in 10⁹. The expectation value range 1e-4 to 1e-6 includes any alignment between a target and query sequence in which the likelihood that such an alignment would occur by chance is in the range from 1 in 10,000 to 1 in 10⁶.

6.7.5 Percent Identity and Percent Homology

One embodiment of the present invention provides a nucleic acid that encodes an engineered protein. The parent protein corresponding to the engineered protein comprises a three-layer swiveling β/β/α domain in which the central beta sheet is parallel and the other beta sheet is antiparallel. Furthermore, at least one portion of the primary sequence of the engineered protein is determined by an engineering scheme. In this embodiment, the nucleic acid comprises a nucleotide sequence that is at least 50%, at least 65%, at least 80%, or at least 90% identical to residues 760 through 1215 of SEQ ID NO: 2. Alternatively, in this embodiment, the nucleic acid comprises a nucleotide sequence that is at least 50%, at least 65%, at least 80%, or at least 90% identical to a nucleotide sequence that is complementary to nucleotides 760 through 1215 of SEQ ID NO: 2.

Sequence identity may be determined using an algorithm such as the BLAST algorithm, described in Altschul et al., J. Mol. Biol. 215, 403-410, (1990) and Karlin et al., PNAS USA 90:5873-5787 (1993). A particularly useful BLAST program is the WU-BLAST-2 program that is described by Altschul et al., Methods in Enzymology, 266:460-480 (1996); http://blast.wustl/edu/blast/REACRCE.html. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A percent amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In one embodiment of the invention, percent (%) nucleic acid sequence identity is defined as the percentage of nucleotide residues in a candidate sequence that are identical with the nucleotide residues of the sequence. A preferred method of computing sequence identity utilizes the BLASTN module of WU-BLAST-2 set to the default parameters, with overlap span and overlap fraction set to 1 and 0.125, respectively. The alignment may include the introduction of gaps in the sequences to be aligned. In addition, for sequences that contain either more or fewer nucleosides than residues 760 through 1215 of SEQ ID NO: 2, it is understood that the percentage of homology is determined based on the number of homologous nucleosides in relation to the total number of nucleosides. Thus, for example, homology of sequences shorter than residues 760 through 1215 of SEQ ID NO: 2 is determined using the number of nucleosides in the shorter sequence.

### 6.8 Libraries

One aspect of the present invention provides a library of engineered proteins. In one embodiment, each engineered protein in the library of engineered proteins comprises a portion of a Group II chaperonin domain that has been subjected to an engineering scheme. In one example, this engineering scheme comprises randomizing at least one portion of the primary sequence of the parent Group II chaperonin domain. In one embodiment, each engineered protein in the library of engineered proteins is an engineering product of the substrate-binding domain of the α subunit of the Thermoplasma acidophilum thermosome (residues 214 through 365 of SEQ ID NO: 1).

Another aspect of the present invention provides a library of engineered proteins in which each engineered protein in the library of engineered proteins comprises a protein having a rubredoxin-like fold (e.g., rubredoxin) that has been subjected to an engineering scheme. A protein that has the rubredoxin-like fold is characterized by a zinc-bound or an iron-bound fold and a primary amino acid sequence that includes two CXnC motifs, where X is a residue of any naturally occurring amino acid and n is an integer in the range of 1 through 4. In typical embodiments at least one portion of the engineered protein is subject to an engineering scheme. This at least one portion of the primary sequence of the engineered protein does not exceed fifty percent of the length of the primary sequence of the engineered protein but is at least five percent of the length of the primary sequence of the engineered protein. In one example, this engineering scheme comprises randomizing at least one portion of the primary sequence of a protein in the rubredoxin-superfamily, (e.g., the rubredoxin family, the desulforedoxin family, or the cytochrome c oxidase subunit F family). In one example, this engineering scheme comprises randomizing rubredoxin (e.g., rubredoxin from Pyrococcus furious). In some embodiments, the parent protein used to derive one or more proteins in the library comprises Pyrococcus furious rubredoxin (SEQ ID NO: 31) and the at least one portion of the primary sequence of each engineered protein in the library of engineered proteins is one or more segments selected from the group consisting of (i) a segment comprising isoleucine 11 of SEQ ID NO: 31; (ii) a segment comprising glycine 17 through glycine 22 of SEQ ID NO: 31; (iii) a segment comprising proline 33 through aspartic acid 35 of SEQ ID NO: 31; (iv) a segment comprising valine 37 of SEQ ID NO: 31; and (v) a segment comprising glycine 42 through serine 46 of SEQ ID NO: 31 (See FIGS. 21 and 22).

In some embodiments, each of the engineered proteins in the library is attached to a genetically replicable package such as a bacteriophage. Suitable bacteriophage include T7, SPbc2, SPP1, phiX174, IEM, T4, UrLamda, P22, M13, f1, P1, MS2, SPO1, B3, HK97, fXo, λ, and λZAP. In some embodiments, a library comprises at least five engineered proteins. In other embodiments, a library comprises at least 25 engineered proteins. In still another embodiment, a library comprises at least 100, at least 500, at least 1000, at least 10⁴, at least 10⁵, at least 10⁶, at least 10⁷, or at least 10⁸ engineered proteins.

Other embodiments of the invention provides a library of proteins that comprises a plurality of engineered proteins. The parent protein that corresponds to each engineered protein in the plurality of engineered proteins comprises a three-layer swiveling β/β/α domain or a protein with a rubredoxin-like fold. In the case where the engineered proteins comprises a three-layer swiveling β/β/α domain, the central beta sheet of the three-layer swiveling β/β/α domain is parallel and the other beta sheet in the three-layer swiveling β/β/α domain is antiparallel. At least one portion of the primary sequence of each engineered protein in the plurality of engineered proteins is determined by an operation of an engineering scheme on the primary sequence of the parent protein. However, the amount of the primary sequence determined by the engineering scheme is subject to constraints. The at least one portion of the primary sequence of the engineered protein does not exceed thirty-five percent, forty percent, forty-five percent, fifty percent, fifty-five percent, sixty percent, sixty-five percent, seventy percent, or seventy-five percent of the length of the primary sequence of the engineered protein. Further, the at least one portion of the primary sequence of the engineered protein comprises at least five percent, ten percent, fifteen percent, twenty percent, twenty-five percent, thirty percent, thirty-five percent, or forty percent of the length of the primary sequence of the engineered protein.

### 6.9 EXAMPLES

6.9.1 Construction of an Engineered Chaperonin Library

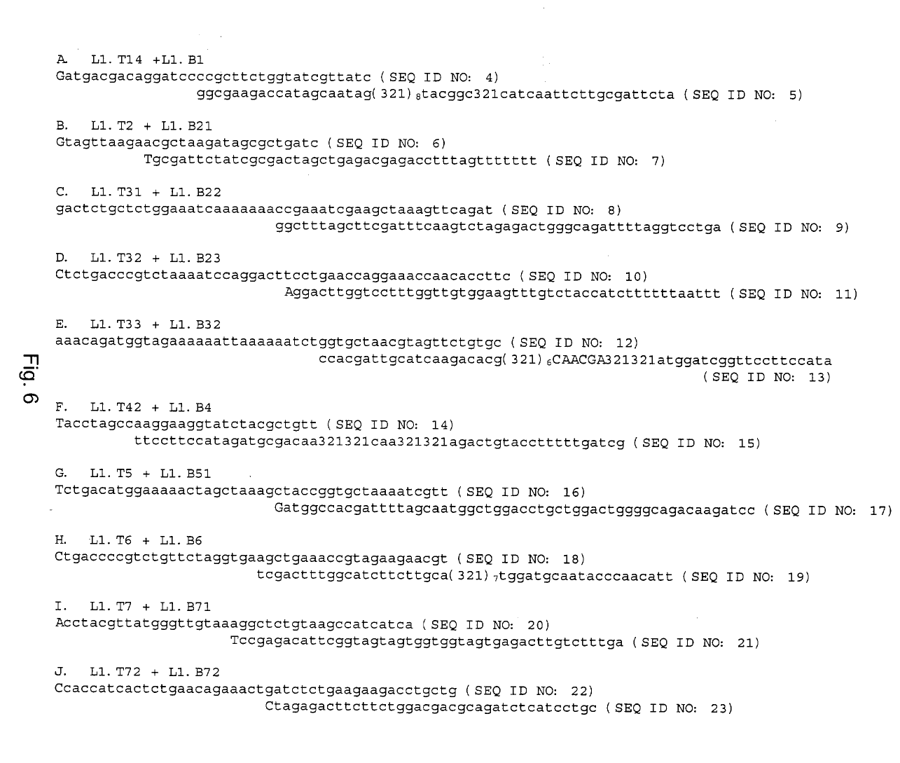

An engineered chaperonin library was constructed from synthetic DNA oligonucleotides by mutually primed extension. Each pair of oligonucleotides illustrated in FIG. 6 was mixed together in a different reaction in order to perform mutually primed extension. Certain positions in the oligonucleotides illustrated in FIG. 6 have degenerate positions. These degenerate positions are denoted by the symbols “1”, “2” and “3”. During the DNA synthesis of the oligonucleotides, a mixture of phosphoramidites were coupled at each indicated position. The molar ratios of the four phosphoramidites that correspond to the symbols “1”, “2” and “3” were as follows:

| | “1” | “2” | “3” |
|---|---|---|---|
| G | 30 | 22 | 19 |
| A | 44 | 14 | 19 |
| T | 0 | 36 | 27 |
| C | 26 | 28 | 35 |

In the thermal reaction that was performed for each pair of oligonucleotides in FIG. 6, the concentration of each of the two primers in the pair was 1.2 μM, the concentration of each dNTP was 200 μM, and the concentration of the Taqplus precision enzyme and buffer were in accordance with the instructions of the manufacturer (Stratagene, La Jolla, Calif.). The following thermal cycling program was run:

| | |
|---|---|
| 1 cycle | 94° C. for one minute |
| 5 cycles | 94° C. for 30 seconds |
| | 55° C. for one minute |
| | 72° C. for 45 seconds |

The library of engineered chaperonin proteins were assembled by mixing together the products of the A+B reaction, C+D reaction, E+F reaction, G+H reaction, and the I+J reaction. The thermal cycling protocol was the same as above, but consisted of eight rather than five cycles. After this, the five resulting reactions were mixed together for an additional 27 cycles in order to form the assembly reaction product. To amplify the expected assembly reaction product, the product was diluted 100-fold into a PCR reaction solution in which the concentration of each of the dNTPs was 200 μM, and the concentration of the Taqplus Precision enzyme and buffer were in accordance with the instructions of the manufacturer (Stratagene, La Jolla, Calif.). This PCR reaction was primed with oligonucleotides L1.T14 and L1.B72, at a concentration of 1 μM each. The following PCR protocol was used to amplify the expected assembly reaction product:

| | |
|---|---|
| 1 cycle | 94° C. for one minute |
| 8 cycles | 94° C. for 30 seconds |
| | 55° C. for one minute |
| | 72° C. for one minute |

A single band of the expected size was observed by agarose gel electrophoresis after this PCR reaction. The predicted sequence of the expected assembly reaction product, shown as only the top strand in the 5′ to 3′ direction, is as follows:

| | |
|---|---|
| GATGACGACAGGATCCCCGCTTCTGGTATCGTTATC45645645645645645645645 | (SEQ ID NO:25) |
| 6ATGCCG456GTAGTTAAGAACGCTAAGATAGCGCTGATCGACTCTGCTCTGGAAATCA | |
| AAAAAACCGAAATCGAAGCTAAAGTTCAGATCTCTGACCCGTCTAAAATCCAGGACTTC | |
| CTGAACCAGGAAACCAACACCTTCAAACAGATGGTAGAAAAAATTAAAAAATCTGGTGC | |
| TAACGTAGTTCTGTGC456456456456456456GTTGCT456456TACCTAGCCAAGG | |
| AAGGTATCTACGCTGTT456456GTT456456TCTGACATGGAAAAACTAGCTAAAGCT | |
| ACCGGTGCTAAAATCGTTACCGACCTGGACGACCTGACCCCGTCTGTTCTAGGTGAAGC | |
| TGAAACCGTAGAAGAACGT456456456456456456456ACCTACGTTATGGGTTGTA | |
| AAGGCTCTGTAAGCCATCATCACCACCATCACTCTGAACAGAAACTGATCTCTGAAGAA | |
| GACCTGCTGCGTCTAGAGTAGGACG | |

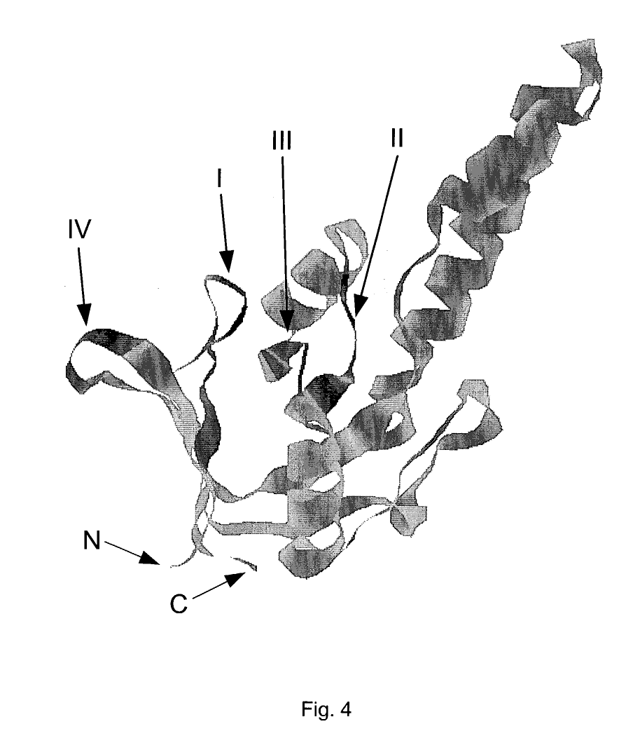

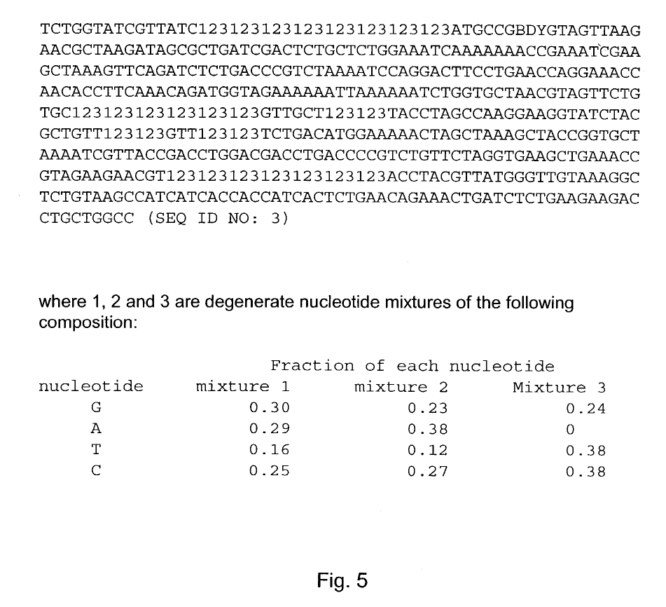

In SEQ ID NO: 25, the symbols “4”, “5” and “6” correspond to degenerate positions, the composition of which is based on the degenerate phosphoramidite mixtures “1”, “2” and “3”, above. SEQ ID NO: 25, translates to:

| | |
|---|---|
| DDDRIPASGIVIXXXXXXXXMPXVVKNAKIALIDSALEIKKTEIEAKVQISDPSKIQDF | (SEQ ID NO:26) |
| LNQETNTFKQMVEKIKKSGANVVLCXXXXXXVAXXYLAKEGIYAVXXVXXSDMEKLAKA | |
| TGAKTVTDLDDLTPSVLGEAETVEERXXXXXXXTYVMGCKGSVSHHHHHHSEQKLISEE | |
| DLLRLE | |

where each symbol “X” in (SEQ ID NO:26) represents a degenerate position. The regions of degeneracy correspond to loops I, II, III, and IV in FIG. 4). The assembly reaction product contains a C-terminal six residue HIS tag as well as a myc tag. It is expected that the majority of the engineered chaperonin library sequences (the assembly reacion product from above) would have defects due to errors in DNA synthesis as well as in-frame stop codons that arise in many of the possible degenerate sequences. Therefore, an attempt was made to remove such defective sequences using preselection measures designed to remove incomplete and/or out of frame engineered chaperonin proteins. This was done by cloning the engineered chaperonin library into an expression vector. Each member of the engineered chaperonin library was cloned into the expression vector in such a manner that a fusion protein would be made between the encoded engineered chaperonin and the protein chloramphenicol acetyltransferase (CAT). The vector that was used, and the conditions for selection, are described in Maxwell et al., Protein Science 8, pp. 1908-1911, 1999. Engineered chaperonin proteins free of frame-shift mutations or premature stop codons will, in the context of this vector, encode library protein-CAT fusion proteins. Therefore, such engineered chaperonin proteins confer chloramphenicol-resistance on the bacteria carrying this plasmid. Engineered chaperonin proteins having frame-shift mutations or premature stop codons will not support bacterial growth on media that includes the antibiotic chloramphenicol.

Individual members of the engineered chaperonin protein library was ligated into the CAT vector (pCFN1) at a ratio of 3:1 insert:vector. A total of 10 μg of ligation product was transformed into ElectroMax DH12S (Gibco Life Technologies, Rockville, Md.) cells and plated onto LB-agar, plates with 50 μg/ml amplicillin and two percent glucose. The complexity of the engineered chaperonin protein library at this stage was 1×10⁷ transformants. The cells were allowed to grow for five hours, after which they were scraped off the plates and a standard DNA miniprep was performed. The DNA was then transformed into JM101 cells (Maxwell et al., Protein Science 8, pp. 1908-1911, 1999) and allowed to grow for thirty minutes in 2XYT/2% glucose media to allow the cells to recuperate. The cells were then diluted into fresh media and allowed to grow for one hour at 37° C. Ampicillin (50 μg/ml) was then added to select for the pCFN1 vector, and 1 mM IPTG was added to allow for expression of the fusion protein. The cells were grown for two more hours at 37° C. The cells were then spread onto LB-agar plates containing 450 μg/ml chloramphenicol/1 mM IPTG and allowed to incubate overnight at 37° C. After this, the cells were scraped from the plate and a standard DNA miniprep was carried out. The complexity of the engineered chaperonin library decreased to 1×10⁵ genes as a result of this preselection.

6.9.2 Recombination of the Engineered Chaperonin Library to Increase Complexity

A recombination process was used to increase the chaperonin library complexity. The recombination process allows for the increase of the engineered chaperonin library described above from 1×10⁵ genes to 1×10¹⁰ genes because the degenerate regions in the engineered chaperonin library become mixed as they recombine with other engineered chaperonins in the library that have different degenerate sequences. To accomplish recombination between individual engineered chaperonin proteins in the library, each engineered chaperonin in the library is amplified in two parts. Part A corresponds roughly to the first half of the chaperonin gene, and part B corresponds roughly to the second half of the chaperonin gene. Parts A and B overlap in one invariant region that has an asymmetric StyI restriction site. After individually amplifying the two halves, library members are cut with StyI, mixed together, and then randomly ligated. Following this, the full-length, ligated product is PCR-amplified.

To amplify part A of each engineered chaperonin in the library, the following primers were used:

| | |
|---|---|
| L1.T1 | |
| 5′-gctacgaattccgcttctggtatcgttatc-3′ | (SEQ ID NO:27) |
| L1.B162 | |
| 5′-agataccttccttggctaggta-3′ | (SEQ ID NO:28) |

To amplify Part B of each engineered chaperonin in the library, the following primers were used:

(SEQ ID NO:29)

| | |
|---|---|
| L1.T132 | 5′-tacctagccaaggaaggtatct-3′ |

(SEQ ID NO:30)

| | |
|---|---|
| L1.B73 | 5′-agcaggataagcttaggccagcaggtcttcttcag-3′ |

It will be appreciated that primers L1.T1 and L1.B162 define part A of the engineered chaperonin and primers L1.T132 and L1.B73 define part B of the engineered chaperonin. In the PCR reaction solution used to respectively amplify parts A and B of the engineered chaperonin, the concentration of each of the dNTPs was 200 μM, the concentration of the Taqplus Precision enzyme and buffer were in accordance with the instructions of the manufacturer (Stratagene, La Jolla, Calif.), and the concentration of each primer was 1 μM. In these PCR reactions, the chaperonin template was the plasmid DNA resulting from minipreps of preselected cells. The concentration of the chaperonin template was approximately 100 pM. The PCR thermalcycler program that was used to amplify parts A and B of the chaperonin library was as follows:

| | |
|---|---|
| 1 cycle | 94° C. for three minutes |
| 20 cycles | 94° C. for one minute |
| | 55° C. for one minute |
| | 72° C. for 45 seconds |
| 1 cycle | 72° C. for six minutes |

After amplification, parts A and B were mixed together in equal concentrations and then purified. The mixture was digested with the StyI enzyme, purified and then religated using T4 DNA ligase. The purified ligation was diluted 500-fold into a PCR reaction and overamplified for twenty cycles using primers L1.T1 and L1.B73. The thermalcycler program was the same as the thermalcycler program used to amplify parts A and B of the chaperonin library. Sequencing of individual clones from the final PCR reaction showed that virtually all of the individual clones were full-length sequences without frame-shift mutations or premature in-frame stop codons.

6.9.3 Construction of an Engineered Rubredoxin Library

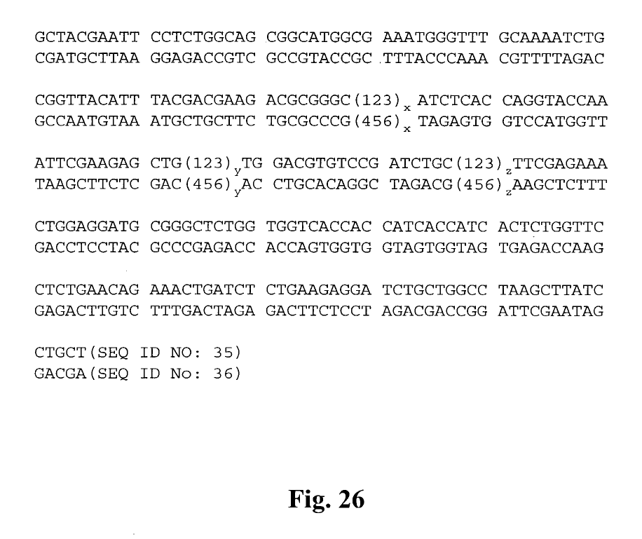

An engineered chaperonin library was constructed from synthetic DNA oligonucleotides. FIG. 26 shows an overall view of the library sequences that were made. Certain positions in the oligonucleotide library illustrated in FIG. 26 have degenerate positions. These degenerate positions are denoted by the symbols “1”, “2”, “3 ”, “4”, “5”, or “6”. During the DNA synthesis of the oligonucleotides, a mixture of phosphoramidites were coupled at each position denoted by the symbols “1”, “2”, “3”, “4”, “5”, or “6”. The normalized molar ratios of the four phosphoramidites (G, A, T, and C) that correspond to the symbols “1”, “2”, “3”, “4”, “5”, and “6” are:

| | “1” | “2” | “3” | “4” | “5” | “6” |
|---|---|---|---|---|---|---|
| G | 24 | 18 | 12 | 33 | 24 | 19 |
| A | 34 | 39 | 0 | 49 | 13 | 17 |
| T | 13 | 10 | 39 | 0 | 34 | 28 |
| C | 29 | 33 | 49 | 18 | 29 | 36 |

Furthermore, as indicated in FIG. 26, regions of degenerate positions were repeated x, y, or z times. Here, x is 5, 9or 14, y is 3 or 6 and z is 6 or 11.

SEQ ID NO: 35 (FIG. 26), translates to:

MAKWVCKICGYIYDEDAG(Z)₁ISPGTKFEEL(Z)₂WTCPIC(Z)₃FEKLED

(SEQ ID NOS: 37, 44, 45, and 46)

where each symbol (Z)₁, (Z)₂ and (Z)₃ represents a variable region introduced using the engineering scheme.

6.9.4 T7 Bacteriophage Preparation

After preparation of an engineered chaperonin library (Sections 6.9.1 and 6.9.2) and a rubredoxin library (Section 6.9.3), the next step was to clone the library into a phage vector so that phage display could be used to select library proteins for their ability to bind to protein targets. Accordingly, in the case of the chaperonin, the library was restricted with EcoRI and HindIII and ligated into the vector T7Select 10-3b, and packaged to form recombinant T7 phage according to the instructions of the manufacturer (Novagen, Madison, Wis.) using an insert to vector ratio of 1:1. In the case of the chaperonin library, the resulting complexity was 6×10⁸ plaque-forming units (pfu).

BLT5615 E. coli cells were grown up in M9LB/50 μg/ml carbenicillin to an OD600 of 0.5. The cells were then induced with 1 mM IPTG for expression of wild-type T7 coat protein. The phage were then added to the cells and allowed to amplify in three liters, which is equivalent to an initial ratio of 1000 cells per pfu. After two hours of growth, the culture was lysed. Then, NaCl was added to a final concentration of 420 mM. The cell debris was removed by centrifugation. Polyethyleneglycol having an average molecular weight of 8000 (PEG8000) was added to a final concentration of 8.3% to precipitate the phage. The phage pellet was then extracted with a total of 36 ml of phage extraction solution (1M NaCl, 10 mM Tris-HCl pH 8.0, 1 mM EDTA) for one hour with occasional vortexing. Then, in the case of the chaperonin library, the product was re-precipitated and re-extracted as above, to yield a final phage solution with a titer of 6×10 pfu/ml.

6.9.5 Identifying Proteins in the Engineered Chaperonin Library that Bind to HP6001 and HP6054

HP6001 and HP6054 are mouse monoclonal antibodies that respectively bind to human IgG1 Fc fragment and human Ig lambda light chain. For each round of selection, the antibody (HP6001 or HP6054) was diluted to 10 μg/ml in 0.1 M Bicarbonate buffer (pH 8.4) and then added to a Nunc Maxisorb 96 well plate for overnight adsorption at 4° C. Unbound protein was washed from the wells. The wells were then blocked with 5% non-fat milk/0.1% BSA in 1×TBST (blocking buffer) for two hours at 37° C. The phage library (dialyzed overnight against 1×TBST) was diluted into blocking buffer and then added to the wells and allowed to incubate at room temperature for one hour. Unbound phage were then washed with 1×TBST. The number of phage added to the target at round one was 6×10¹¹. About 10⁹ phage were added in subsequent rounds. A total of five rounds were performed. The number of washes and wells in each round was as follows:

| | Number of | Number of |
|---|---|---|
| Round | wells | washes |
| 1 | 16 | 4 |
| 2 | 16 | 4 |
| 3 | 3 | 11 |
| 4 | 3 | 11 |
| 5 | 3 | 11 |

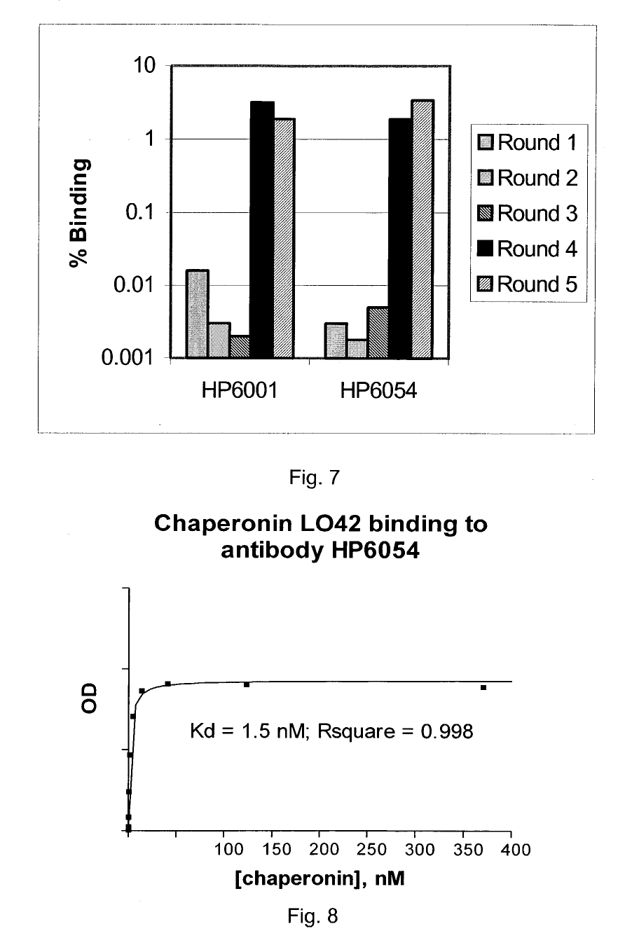

After the wells had been washed in each round as described above, BLT5615 cells were added to the wells and allowed to incubate. More specifically, BLT5615 cells were induced at an OD600 of 0.5 and allowed to grow for thirty minutes, as described in the protocol for the T7 system (Novagen). The induced cells were then added to the washed wells (200 μl/well) and allowed to incubate at 37° C. for thirty minutes with mixing every five minutes. At this point, the cells were removed from the wells and placed in a 14 ml culture tube. Lysis was induced by shaking the cells at 37° C. Also, one well in each round was always eluted with 1% SDS in 1×TBST for titering (no bacteria were added to this well). The percent of phage binding under these conditions as a function of selection round is shown in FIG. 7.

Because the selection profile for engineered chaperonin proteins that bind to HP6001 and engineered chaperonin proteins that bind to HP6054 (FIG. 7) looked very similar, only the engineered chaperonin proteins that bind to HP6054 were characterized. Individual phage from rounds three and four of the HP6054-binding experiments were screened by ELISA. In the ELISA, the target protein (HP6054) was immobilized onto 96 plate wells in the same manner as described above. Then, phage lysates from individual clones were incubated in the wells in blocking buffer (5% non-fat milk/0.1% BSA/1×TBST) for one hour at room temperature. Unbound phage were washed away. Then an anti-T7 tag antibody, conjugated to horse radish peroxidase, was added at 1500-fold dilution. After fifteen minutes, the wells were again washed. TMB HRP substrate (KPL, Gaitherburg, Md.) was then added. HRP activity was monitored by the appearance of a blue-colored product resulting from degradation of the substrate. The reaction was stopped with 1M phosphoric acid. Using this method, 71 clones that bound to the target were identified.

DNA fingerprinting was then performed to determine how many unique sequences were represented among the 71 clones. DNA fingerprinting is the digestion of a clone with a mixture of restriction enzymes in order to generate a banding pattern on a gel that is characteristic of the clone DNA sequence. Sequences that were determined to be unique by DNA fingerprinting were cloned into an expression vector that contains a FLAG-tag upstream of the insertion site. Then, the clones were expressed in E. coli as free proteins (i.e. not attached to phage). The expressed proteins were purified using the HIS-tag. Purified proteins were then tested for the ability to bind to HP6054.

To test whether the purified protein bound to the desired target, an ELISA assay was performed using the same conditions described above with the exception that purified protein rather than phage was added to the wells. Further, the developing antibody was an anti-FLAG-HRP conjugate (Sigma, St. Louis, Mo.). Four different engineered chaperonin proteins that specifically bound to HP6054 were identified in this way.

An affinity ELISA was performed on the four engineered chaperonin proteins that specifically bind to HP6054. In these assays, the engineered chaperonin protein was serially diluted and the ELISA signal was plotted as a function of engineered chaperonin protein concentration. The EC₅₀ is the chaperonin protein concentration that gives 50% of the maximum saturated signal. The following EC₅₀ values for the four library proteins were measured:

| Clone name | EC50 | |
|---|---|---|
| LO6 | 110 | nM |
| LO42 | 1.5 | nM |
| LO43 | 3.5 | μM |
| 311-27-8 | 30 | μM |

An example of a binding curve that was performed using this ELISA assay is shown in FIG. 8 for the engineered chaperonin protein LO42.

6.9.6 Identifying Proteins in the Chaperonin Library that Bind to Human Chorionic Gonadotropin

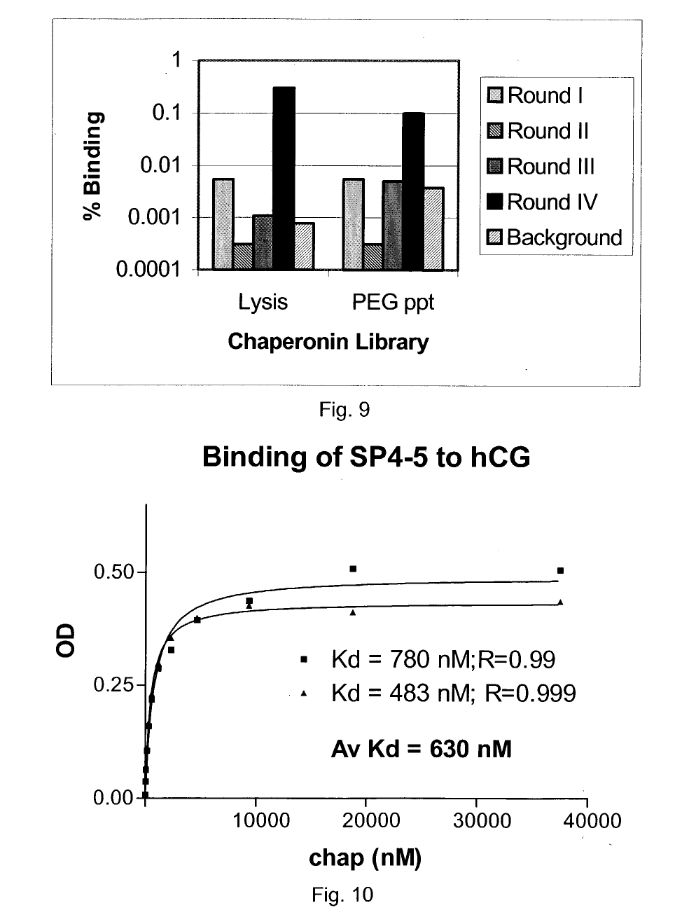

The methods used to identify engineered chaperonin proteins that bind to human chorionic gonadotropin (hCG) were the same as the methods used to identify engineered chaperonin proteins that bind to HP6001 and HP6054, with a few minor differences. One difference was that the hCG target was biotinylated using an NHS-SS-biotin reagent (Pierce Chemical, Rockford, Ill.). The target was then immobilized onto Neutravidin-coated or Streptavidin-coated strip plates (Pierce), with alternating (between rounds of selection) 5% non-fat milk or 3% BSA Blocking Buffer. Another difference was that after phage incubation with the target and subsequent washing, 50 μl of media was added and incubated with shaking for thirty minutes at 37° C., potentially releasing some of the bound phage. BLT5615 cells were then added as mentioned above. The final difference was that a comparison was made between the method used in previously described selections with a method in which the phage were PEG-precipitated after each round of amplification. This comparison between PEG precipitated and non-PEG precipitated phage selections was made throughout the entire selection. The results of these two selections is shown in FIG. 9.

A total of 192 plaques were screened from the PEG precipitated and non-PEG precipitated eluted phage of round three (FIG. 9). Screening at the phage and protein level was similar to that for the HP6054 selections. The screen identified three engineered chaperonin proteins that were solubly expressed in E. coli and could bind hCG. Both the PEG-precipitated and non-PEG-precipitated selections gave similar results. In these affinity ELISA assays, the engineered chaperonin was serially diluted and the ELISA signal was plotted as a function of engineered chaperonin concentration to determine EC₅₀s, as above. The following EC₅₀ values for the three engineered library proteins was measured:

| Clone name | EC50 | |
|---|---|---|
| SP4-1 | 5.4 | μM |
| SP4-2 | 48 | μM |
| SP4-5 | 630 | nM |

An example of a binding curve that was performed using this ELISA assay is shown in FIG. 10 for the engineered chaperonin SP4-5.

| | |
|---|---|
| The amino acid sequence of SP4-1 is: | |
| DDDRIPASGIVIDRVGRDSNMPHVVKNAKIALIDSALEIKKTEIEAKVQISDPSKIQDF | (SEQ ID NO:41) |
| LNQETNTFKQMVEKIKKSGANVVLCADDYAHVAYDYLAKEGIYAVGYVTESDMEKLAKA | |
| TGAKIVTDLDDLTPSVLGEAETVEERPWGNNKETYVMGCKGSVSHHHHHHSEQKLISEE | |
| DLLRLE | |
| The amino acid sequence of SP4-2 is: | |
| DDDRIPASGIVIVGHNKVPSMPRVVKNAKIALIDSALEIKKTEIEAKVQISDPSKIQDF | (SEQ ID NO:42) |
| LNQETNTFKQMVEKIKKSGANVVLCGYKNLTVAYEYLAKEGIYAVYHVDESDMEKLAKA | |
| TGAKIVTDLDDLTPSVLGEAETVEERGTANAPATYVMGCKGSVSHHHHHHSEQKLISEE | |
| DLLRLE | |
| The amino acid sequence of SP4-5 is: | |
| DDDRIPASGIVIARPGESAFMPDVVKNAKIALIDSALEIKKTEIEAKVQISDPSKIQDF | (SEQ ID NO:43) |
| LNQETNTFKQMVEKIKKSGANVVLCNGDQADVAYDYLAKEGIYAVHFVDESDMEKLAKA | |
| TGAKIVIDLDDLTPSVLGEAETVEERKYTEGVPTYVMGCKGSVSHHHHHHSEQKLISEE | |
| DLLRLE | |

6.9.7 Identifying Proteins in the Rubredoxin Library that Bind to Human Chorionic Gonadotropin

The method used to identify engineered rubredoxin proteins that bind to human chorionic gonadotropin (hCG) were the same as the method used to identify chaperonin proteins that bind to hCG described in Section 6.9.6, above. The method identified three engineered rubredoxin proteins that could bind hCG. In the affinity ELISA assays, the engineered rubredoxin was serially diluted and the ELISA signal was plotted as a function of engineered rubredoxin concentration to determine EC₅₀s, as above. The following EC₅₀ values for the three library proteins was measured:

| Clone name | EC50 | |
|---|---|---|
| G3 | 81 | nM |
| D7 | 4 | μM |
| F3 | 35 | nM |

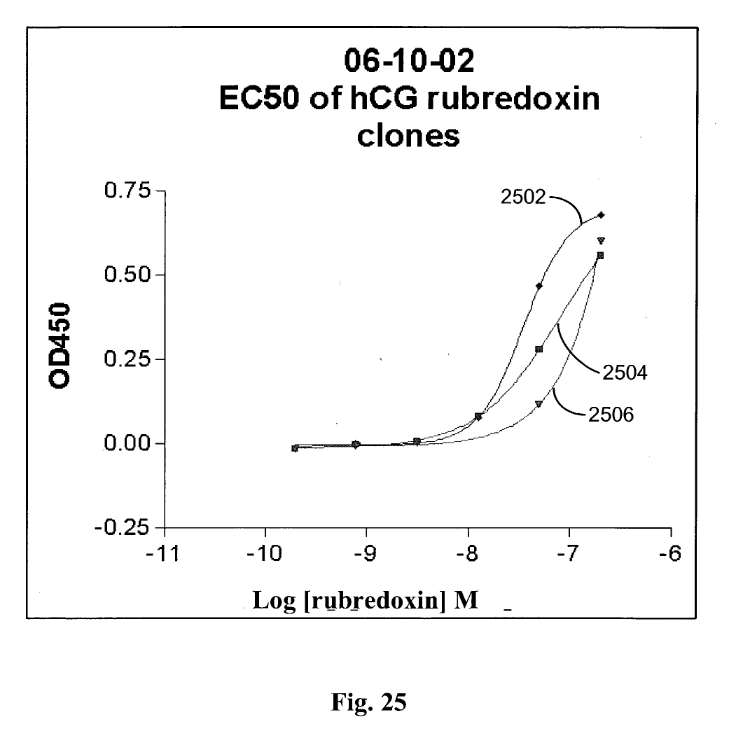

The binding curves obtained from the ELISA assays are shown in FIG. 25.

The primary amino acid sequence of G3 is:

MAKWVCKICGYIYDEDAGFVEYYGHPNISPGTKFEELSRYVDGWTCPICSYGMPIFEKL ED (SEQ ID NO: 38)

where italicized regions represent engineered regions.

The primary amino acid sequence of D7 is:

MAKWVCKICGYIYDEDAGEDPGHRSRYISPGTKFEELTTGWTCPICRCTNSTTSTNC FEKLED (SEQ ID NO: 39)

where italicized regions represent engineered regions.

The primary amino acid sequence of F3 is:

MAKWVCKICGYIYDEDAGFVEYYDDGVISPGTKFEELHTAWTCPICSCTYNPFEKLED (SEQ ID NO: 40)

where underlined regions represent engineered regions.

6.9.8 Identifying Proteins in the Chaperonin Library that Bind to Human Leptin

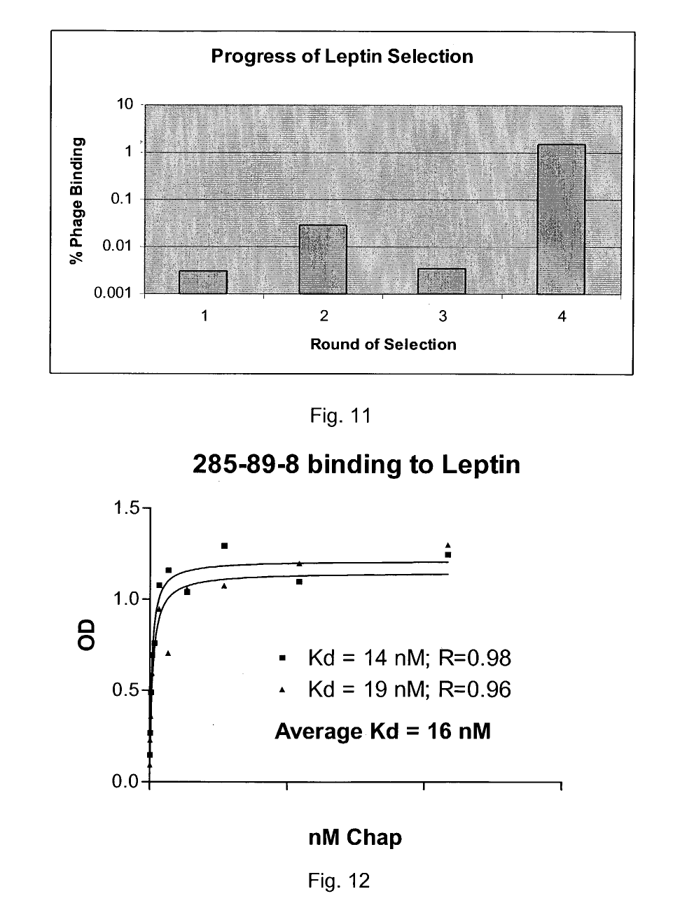

The selection against human leptin was as described for hCG, above, unless for the following changes. After incubation of the target with the phage library and subsequent washing, bound phage was eluted by the addition of 50 mM dithiothreitol (DTT) in 1×TBST/0.05% BSA for thirty minutes at 37° C. with agitation. This elution removes the target protein 96 plate well since there is a disulfide bond in the biotinylation reagent. The eluted phage was then passed over a sephadex de-salting column and amplified in a shaking culture tube at 37° C. until lysis. The results from this selection is shown in FIG. 11.

A total of 296 plaques were screened from phage eluted during round three. Screening at the phage and protein levels was similar to that for the HP6054 selections previously described. The screen identified three library proteins that were solubly expressed in E. coli and that could bind leptin. Affinity ELISA gave the following EC50's:

| Clone name | EC50 |
|---|---|
| 285-63-4 | 670 nM |
| 258-89-2 | 81 nM |
| 285-89-8 | 16 nM |

An example of a binding curve that was performed using this ELISA assay is shown in FIG. 12 for the engineered chaperonin 285-89-8.

### 7. REFERENCES CITED

All references cited herein are incorporated herein by reference in their entirety and for all purposes to the same extent as if each individual publication or patent or patent application was specifically and individually indicated to be incorporated by reference in its entirety for all purposes.

What is claimed is:

1. An engineered protein, wherein the parent protein that corresponds to said engineered protein comprises a three-layer swiveling β/β/α domain, wherein the central beta sheet of said three-layer swiveling β/β/α domain is parallel and the other beta sheet in said three-layer swiveling β/β/α domain is antiparallel, and
wherein at least one portion of the primary sequence of said engineered protein is determined by an operation of an engineering scheme on the primary sequence of said parent protein, with the provisos that:
(i) said at least one portion of the primary sequence of said engineered protein that is determined by the operation of the engineering scheme on the primary sequence of the parent protein does not exceed fifty percent of the length of the primary sequence of the engineered protein; and
(ii) said at least one portion of the primary sequence of said engineered protein that is determined by the operation of the engineering scheme on the primary sequence of the parent protein comprises at least five percent of the length of the primary sequence of the engineered protein.

2. The engineered protein of claim 1, wherein said engineered protein is attached to a surface.

3. The engineered protein of claim 1, wherein said engineered protein is attached to a chip, slide or bead.

4. The engineered protein of claim 1, wherein the operation of the engineering scheme comprises wholly or partly randomizing at least one portion of the primary sequence of the parent protein in order to form said engineered protein.

5. The engineered protein of claim 1, wherein the operation of the engineering scheme comprises altering at least one portion of the primary sequence of the parent protein using a rational scheme in order to form said engineered protein.

6. The engineered protein of claim 1, wherein said engineered protein has the ability to bind to a compound that the parent protein does not bind.

7. The engineered protein of claim 1, wherein said three-layer swiveling β/β/α domain has a β-sandwich architecture comprising a first β sheet and a second β sheet, wherein said first β sheet is approximately orthogonal to said second β sheet, the first β sheet having a βα βα βα topology and the first β sheet flanked on its exterior face by two antiparallel helices.

8. The engineered protein of claim 1, wherein said parent protein comprises the substrate-binding domain of a chaperonin.

9. The engineered protein of claim 1, wherein said parent protein comprises the substrate-binding domain of a Group II chaperonin.

10. The engineered protein of claim 1, wherein said parent protein comprises the substrate-binding domain of the α or β subunit of the Thermoplasma acidophilum thermosome.

11. The engineered protein of claim 1, wherein said parent protein comprises residue 214 through residue 365 of SEQ ID NO: 1 and said at least one portion of said primary sequence includes any combination of:
(i) a segment comprising aspartic acid 219 through lysine 226 of SEQ ID NO: 1;
(ii) a segment comprising glutamine 291 (Gln 291) through histine 300 of SEQ ID NO: 1;
(iii) a segment comprising arginine 311 through lysine 315 of SEQ ID NO: 1; and
(iv) a segment comprising lysine 351 through methionine 357 of SEQ ID NO: 1.

12. The engineered protein of claim 1, wherein said engineered protein is free of disulfide bonds.

13. The engineered protein of claim 1, wherein said engineered protein is part of a fusion protein.

14. A composition comprising the engineered protein of claim 1 and a physiologically-acceptable carrier.

15. The engineered protein of claim 1, wherein said at least one portion of the primary sequence of said engineered protein collectively is less than twenty percent of the total sequence of said engineered protein.

16. The engineered protein of claim 1, wherein said operation of said engineering scheme results in an increase or decrease in the overall number of residues present in the engineered protein relative to the number of residues present in the parent protein.

17. The engineered protein of claim 6, wherein said compound is a hormone, a low molecular weight compound, a peptide, a protein, or an oligonucleotide.

18. The engineered protein of claim 6, wherein, when said engineered protein is attached to a surface using N-terminal or C-terminal chemistry, the engineered protein retains the ability to bind to said compound.

19. The engineered protein of claim 6, wherein said engineered protein exhibits an EC₅₀ for said compound that is greater than 1×10³ (M⁻¹) and said corresponding parent protein exhibits an EC₅₀ for said compound that is less than 1×10³ (M⁻¹).

20. The engineered protein of claim 1, wherein each said portion of the primary sequence of said engineered protein that is determined by the operation of the engineering scheme corresponds to a solvent-exposed region of said parent protein.

21. The engineered protein of claim 1, wherein said at least one portion of the primary sequence of said engineered protein that is determined by an engineering scheme contains one or more amino acid residue positions that are identical to the corresponding residues in the parent protein.

22. The engineered protein of claim 1, wherein said three-layer swiveling β/β/α domain has an N-terminus and a C-terminus, and wherein said N-terminus or said C-terminus, or both, is attached to an affinity tag.

23. The engineered protein of claim 1, wherein the N-terminal portion of said engineered protein includes a serine residue or a threonine residue and said engineered protein is attached to a surface by selectively oxidizing said serine residue or said threonine residue to form a glyoxylyl group or a keto group that is then reacted with a functionality on said surface.

24. The engineered protein of claim 23, wherein said functionality is an aminooxy or a hydrazine functionality.

25. The engineered protein of claim 23, wherein said functionality is provided by a heterobifunctional compound, said heterobifunctional compound bearing both an aminooxy- or a hydrazine-functionality and a second reactive group that attaches to said surface.

26. The engineered protein of claim 1, wherein the N-terminal portion of said engineered protein includes a cysteine residue and said engineered protein is attached to a surface by selectively derivatizing said cysteine reside by reacting it with a thioester functionality on said surface.

27. The engineered protein of claim 26, wherein said thioester functionality is provided by a heterobifunctional compound, said heterobifunctional compound bearing both a thioester functionality and a second reactive group that attaches to said surface.

28. A nucleic acid encoding the engineered protein of claim 1.

29. The nucleic acid of claim 28, wherein said nucleic acid is DNA.

30. The nucleic acid of claim 28, comprising a nucleotide sequence that hybridizes under conditions of high stringency to nucleotides 760 through 1215 of SEQ ID NO: 2 or a nucleotide sequence that hybridizes under conditions of high stringency to a polynucleotide that is complementary to nucleotides 760 through 1215 of SEQ ID NO: 2.

31. The nucleic acid of claim 28, comprising a nucleotide sequence that hybridizes under conditions of moderate stringency to nucleotides 760 through 1215 of SEQ ID NO: 2 or a nucleotide sequence that hybridizes under conditions of moderate stringency to a polynucleotide that is complementary to nucleotides 760 through 1215 of SEQ ID NO: 2.

32. The nucleic acid of claim 28, comprising a nucleotide sequence that is at least 50% identical to residues 760 through 1215 of SEQ ID NO: 2 or is at least 50% identical to a nucleotide sequence that is complementary to nucleotides 760 through 1215 of SEQ ID NO: 2.

33. The nucleic acid of claim 28, comprising a nucleotide sequence that is at least 65% identical to residues 760 through 1215 of SEQ ID NO: 2 or is at least 65% identical to a nucleotide sequence that is complementary to nucleotides 760 through 1215 of SEQ ID NO: 2.

34. The nucleic acid of claim 28, comprising a nucleotide sequence that is at least 80% identical to residues 760 through 1215 of SEQ ID NO: 2 or is at least 80% identical to a nucleotide sequence that is complementary to nucleotides 760 through 1215 of SEQ ID NO: 2.

35. The nucleic acid of claim 28, comprising a nucleotide sequence that is at least 90% identical to residues 760 through 1215 of SEQ ID NO: 2 or is at least 90% identical to a nucleotide sequence that is complementary to nucleotides 760 through 1215 of SEQ ID NO: 2.

36. An array comprising a plurality of engineered proteins immobilized on a solid support, wherein each engineered-protein in the array of engineered proteins corresponds to a parent protein that comprises a three-layer swiveling β/β/α domain, wherein the central beta sheet of said three-layer swiveling β/β/α domain is parallel and the other beta sheet in said three-layer swiveling β/β/α domain is antiparallel; and
wherein at least one portion of the primary sequence of each said engineered protein in said plurality of engineered proteins is determined by an operation of an engineering scheme on the primary sequence of said corresponding parent protein, with the provisos that:
(i) said at least one portion of the primary sequence of each said engineered protein in said plurality of engineered proteins that is determined by the operation of the engineering scheme on the primary sequence of the corresponding parent protein does not exceed fifty percent of the length of the primary sequence of the engineered protein; and
(ii) said at least one portion of the primary sequence of each said engineered protein in said plurality of engineered proteins that is determined by the operation of the engineering scheme on the primary sequence of the corresponding parent protein comprises at least five percent of the length of the primary sequence of the engineered protein.

37. The array of claim 36, wherein said parent protein comprises a chaperonin.

38. The array of claim 36, wherein at least one engineered protein in said array of engineered proteins is characterized by an ability to bind to a compound that the parent protein does not bind.

39. The array of claim 36, wherein said compound is a protein, a hormone, a low molecular weight compound, a peptide, or an oligonucleotide.

40. The array of claim 36, wherein said parent protein comprises the substrate-binding domain of a chaperonin.

41. The array of claim 36, wherein said parent protein comprises the substrate-binding domain of a Group II chaperonin.

42. The array of claim 36, wherein said parent protein comprises the substrate-binding domain of the α or β subunit of the Thermoplasma acidophilum thermosome.

43. The array of claim 36, wherein said parent protein comprises residue Ser 214 through residue Asn 365 of the α subunit of the Thermoplasma acidophilum thermosome (residue 214 to residue 365 of SEQ ID NO: 1) and wherein said at least one portion of said primary sequence includes any combination of:
(i) a segment comprising aspartic acid 219 through lysine 226 of SEQ ID NO: 1;
(ii) a segment comprising glutamine 291 (Gln 291) through histine 300 of SEQ ID NO: 1;
(iii) a segment comprising arginine 311 through lysine 315 of SEQ ID NO: 1; and
(iv) a segment comprising lysine 351 through methionine 357 of SEQ ID NO: 1.

44. The array of claim 36, wherein said solid support is a bead, a slide or chip.

45. A method of determining whether an engineered protein binds to a compound, wherein the parent protein that corresponds to said engineered protein comprises a three-layer swiveling β/β/α domain, wherein the central beta sheet of said three-layer swiveling β/β/α domain is parallel and the other beta sheet in said three-layer swiveling β/β/α domain is antiparallel, and
wherein at least one portion of the primary sequence of said engineered protein is determined by an operation of an engineering scheme on the primary sequence of said parent protein, with the provisos that:
(i) said at least one portion of the primary sequence of said engineered protein that is determined by the operation of the engineering scheme on the primary sequence of the parent protein does not exceed fifty percent of the length of the primary sequence of the engineered protein; and
(ii) said at least one portion of the primary sequence of said engineered protein that is determined by the operation of the engineering scheme on the primary sequence of the parent protein comprises at least five percent of the length of the primary sequence of the engineered protein; the method comprising contacting said engineered protein with said compound.

46. The method of claim 45, wherein said engineered protein is attached to a solid support.

47. The method of claim 45, wherein said solid support is a bead, a slide or a chip.

48. The method of claim 45, wherein said engineered protein forms a complex with said compound and wherein an EC₅₀ of said complex is less than 10⁻⁶ moles/liter.

49. A method for using an engineered protein, the method comprising:
(a) contacting a compound with an array of candidate engineered proteins immobilized on a solid support, the array of engineered proteins immobilized on the solid support including said engineered protein, each said engineered protein in said array of engineered proteins comprising an engineered chaperonin domain, wherein at least one portion of the primary sequence of said engineered chaperonin domain is determined by an engineering scheme, with the provisos that
(i) said at least one portion of the primary sequence of said engineered chaperonin domain is greater than five percent of the primary sequence of said engineered chaperonin domain; and
(ii) said at least one portion of the primary sequence of said engineered chaperonin domain is less than fifty percent of the primary sequence of said engineered chaperonin domain; and
(b) determining whether said engineered protein binds to said compound.

50. The method of claim 49, said method further comprising the steps of:
(c) further engineering said engineered protein that binds to said compound in step (b);
(d) forming an array on a solid support with the further engineered proteins of step (c); and
(e) repeating step (a) and step (b) using, in step (a), the array of further engineered proteins as said array of candidate engineered proteins.

51. A method for detecting a compound in a sample, the method comprising contacting said sample with an engineered protein that binds to the compound, wherein
the parent protein that corresponds to said engineered protein comprises a three-layer swiveling β/β/α domain, wherein the central beta sheet of said three-layer swiveling β/β/α domain is parallel and the other beta sheet in said three-layer swiveling β/β/α domain is antiparallel, and wherein
at least one portion of the primary sequence of said engineered protein is determined by an operation of an engineering scheme on the primary sequence of said parent protein, with the provisos that:
(i) said at least one portion of the primary sequence of said engineered protein that is determined by the operation of the engineering scheme on the primary sequence of the parent protein does not exceed fifty percent of the length of the primary sequence of the engineered protein; and
(ii) said at least one portion of the primary sequence of said engineered protein that is determined by the operation of the engineering scheme on the primary sequence of the parent protein comprises at least five percent of the length of the primary sequence of the engineered protein.

52. The method of claim 51, the method further comprising detecting a complex between said engineered protein and said compound.

53. The method of claim 51, wherein said parent domain comprises the substrate-binding domain of the α or β subunit of a chaperonin.

54. The method of claim 51, wherein said sample is a biological sample.

55. The method of claim 51, wherein said engineered protein is immobilized on a bead, a slide or a chip.

56. The method of claim 51, wherein said engineered protein is immobilized on said solid support as part of an array of engineered proteins.

57. The method of claim 51, wherein said compound is a protein.

58. The method of claim 51, wherein said parent protein comprises a Group II chaperonin.

59. The method of claim 51, wherein said parent protein comprises a portion of a Thermoplasma acidophilum thermosome.

60. The method of claim 51, wherein the parent protein comprises Ser 214 through Asn 365 of the α subunit of the Thermoplasma acidophilum thermosome (residue 214 through residue 365 of SEQ ID NO: 1) and said at least one portion of said primary sequence of said engineered protein that is determined by an engineering scheme includes any combination of:
(i) a segment comprising aspartic acid 219 through lysine 226 of SEQ ID NO: 1;
(ii) a segment comprising glutamine 291 (Gln 291) through histine 300 of SEQ ID NO: 1;
(iii) a segment comprising arginine 311 through lysine 315 of SEQ ID NO: 1; and
(iv) a segment comprising lysine 351 through methionine 357 of SEQ ID NO: 1.

61. The method of claim 51, wherein a complex between said engineered protein and the compound is detected by spectroscopy, radiography, fluorescence detection, mass spectrometry, luminescence, or surface plasmon resonance.

62. The method of claim 61, wherein the EC₅₀ of the complex is less than 10⁻⁶ moles/liter.

63. A mutated chaperonin protein, wherein one or more portions of the mutated chaperonin polypeptide vary by engineering of at least ten amino acids from the corresponding portion of the wild-type chaperonin substrate-binding domain and wherein the sequence of the mutated chaperonin protein has at least 50% total amino acid sequence identity with the wild-type chaperonin substrate-binding domain.

64. The mutated chaperonin protein of claim 63, wherein the mutated chaperonin protein is capable of binding to a compound to form a complex, comprising the mutated chaperonin protein and the compound, having a dissociation constant of less than 10⁻⁶ moles/liter.

65. A nucleic acid molecule encoding the mutated chaperonin protein of claim 64.

66. An expression vector comprising an expression cassette operably linked to the nucleic acid molecule of claim 65.

67. A host cell comprising the expression vector of claim 66.

68. A method of preparing an engineered chaperonin binding domain library from a set of paired oligonucleotides, wherein the first oligonucleotide in each pair of oligonucleotides includes a region that is complementary to the corresponding second oligonucleotide in each pair of oligonucleotides, and wherein at least one oligonucleotide in the set of paired oligonucleotides includes a randomized sequence, the method comprising:
(a) mixing together, in a different reaction, each pair of paired oligonucleotides in the set of oligonucleotides and performing mutually primed DNA synthesis using a DNA polymerase;
(b) mixing the reaction products of step (a) and performing multiple cycles of denaturation, annealing, and DNA synthesis using a DNA polymarase; and
(c) amplifying the DNA constructs from step (b) encoding full-length chaperonin domain library members; and
(d) cloning the product of step (c) into an expression vector.

69. A library of proteins that comprises a plurality of engineered proteins, wherein the parent protein that corresponds to each engineered protein in said plurality of engineered proteins comprises a three-layer swiveling β/β/α domain, wherein the central beta sheet of said three-layer swiveling β/β/α domain is parallel and the other beta sheet in said three-layer swiveling β/β/α domain is antiparallel, and
wherein at least one portion of the primary sequence of each engineered protein in said plurality of engineered proteins is determined by an operation of an engineering scheme on the primary sequence of said parent protein, with the provisos that:
(i) said at least one portion of the primary sequence of said engineered protein that is determined by the operation of the engineering scheme on the primary sequence of the parent protein does not exceed fifty percent of the length of the primary sequence of the engineered protein; and
(ii) said at least one portion of the primary sequence of said engineered protein that is determined by the operation of the engineering scheme on the primary sequence of the parent protein comprises at least five percent of the length of the primary sequence of the engineered protein.

70. The library of claim 69, wherein the parent protein comprises a Group II chaperonin.

71. The library of claim 69, wherein the parent protein comprises the substrate-binding domain of the α or β subunit of the Thermoplasma acidophilum thermosome.

72. The library of claim 69, wherein the parent protein comprises Ser 214 through Asn 365 of the α subunit of the Thermoplasma acidophilum thermosome (residue 214 through residue 365 of SEQ ID NO: 1) and wherein each said at least one portion of the primary sequence of each engineered protein in said library of engineered proteins is selected from the group consisting of:
(i) a segment comprising aspartic acid 219 through lysine 226 of SEQ ID NO: 1;
(ii) a segment comprising glutamine 291 (Gln 291) through histine 300 of SEQ ID NO: 1;
(iii) a segment comprising arginine 311 through lysine 315 of SEQ ID NO: 1; and
(iv) a segment comprising lysine 351 through methionine 357 of SEQ ID NO: 1.

73. The library of claim 69, wherein each of said engineered proteins in said plurality of engineered proteins is attached to a genetically replicable package.

74. The library of claim 69, wherein the genetically replicable package is a bacteriophage.

75. The library of claim 69, wherein the bacteriophage is T7, SPbc2, SPP1, phiX174, IEM, T4, UrLamda, P22, M13, f1, P1, MS2, SPO1, B3, HK97, fXo, or λ.

76. An expression vector comprising the nucleic acid of claim 28.

77. A host cell comprising the nucleic acid of claim 28.

78. A method of making an engineered protein, the method comprising subjecting at least one portion of the primary sequence of a parent protein to an engineering scheme in order to produce said engineered protein, with the provisos that:
(i) said parent protein comprises a three-layer swiveling β/β/α domain, wherein the central beta sheet of said three-layer swiveling β/β/α domain is parallel and the other beta sheet in said three-layer swiveling β/β/α domain is antiparallel;
(ii) said at least one portion of the primary sequence of said engineered protein does not exceed fifty percent of the length of the primary sequence of said engineered protein; and
(iii) said at least one portion of the primary sequence of said engineered protein comprises at least five percent of the length of the primary sequence of said engineered protein.

79. The method of claim 78, wherein said engineering scheme is a pseudo-randomization scheme and the step of subjecting said at least one portion of the primary sequence of said parent protein to an engineering scheme results in the randomization of said at least one portion of the primary sequence.

80. The method of claim 78, wherein said engineering scheme is a randomization scheme and the step of subjecting said at least one portion of the primary sequence of said parent protein to an engineering scheme results in the pseudo-randomization of said at least one portion of the primary sequence.

81. An engineered protein, wherein the parent protein that corresponds to said engineered protein has a zinc-bound fold or an iron-bound fold and the primary sequence of the parent protein includes two CXₙC motifs, wherein X is a residue of any naturally occurring amino acid and n is 1, 2, 3 or 4, and
wherein at least one portion of the primary sequence of said engineered protein is determined by an operation of an engineering scheme on the primary sequence of said parent protein, with the provisos that:
(i) said at least one portion of the primary sequence of said engineered protein that is determined by the operation of the engineering scheme on the primary sequence of the parent protein does not exceed fifty percent of the length of the primary sequence of the engineered protein; and
(ii) said at least one portion of the primary sequence of said engineered protein that is determined by the operation of the engineering scheme on the primary sequence of the parent protein comprises at least five percent of the length of the primary sequence of the engineered protein.

82. The engineered protein of claim 81, wherein said engineered protein is attached to a surface.

83. The engineered protein of claim 81, wherein said engineered protein is attached to a chip, slide or bead.

84. The engineered protein of claim 81, wherein the operation of the engineering scheme comprises wholly or partly randomizing at least one portion of the primary sequence of the parent protein in order to form said engineered protein.

85. The engineered protein of claim 81, wherein the operation of the engineering scheme comprises altering at least one portion of the primary sequence of the parent protein using a rational scheme in order to form said engineered protein.

86. The engineered protein of claim 81, wherein said engineered protein has the ability to bind to a compound that the corresponding parent protein does not bind.

87. The engineered protein of claim 86, wherein said compound is a hormone, a low molecular weight compound, a peptide, a protein, or an oligonucleotide.

88. The engineered protein of claim 86, wherein, when said engineered protein is attached to a surface using N-terminal or C-terminal chemistry, the engineered protein retains the ability to bind to said compound.

89. The engineered protein of claim 86, wherein said engineered protein exhibits an EC₅₀ for said compound that is greater than 1×10³ (M⁻¹) and said parent protein exhibits an EC₅₀ for said compound that is less than 1×10³ (M⁻¹).

90. The engineered protein of claim 81, wherein said parent protein is in the rubredoxin-superfamily.

91. The engineered protein of claim 81, wherein said parent protein is in the rubredoxin family, the desulforedoxin family, or the cytochrome c oxidase subunit F family.

92. The engineered protein of claim 81, wherein said parent protein comprises rubredoxin.

93. The engineered protein of claim 81, wherein an N-terminal portion of the primary sequence of the parent protein includes an alanine at a position n, a tryptophan at a position n+2, a glutamic acid at a position n+13, and a phenylalanine at a position n+28.

94. The engineered protein of claim 81, wherein the parent protein has an overall shape that is ellipsoidal and comprises a three-stranded antiparallel β-sheet with a hydrophobic core comprising a plurality of aromatic residues.

95. The engineered protein of claim 81, wherein said parent protein comprises rubredoxin from Pyrococcus furiousus, Desulfovibrio gigas, Pseudomonas oleovorans, Clostridium pasteurianum, Desulfovibrio vulgaris, Desulfovibrio desulfuricans, or Guillardia theta.

96. The engineered protein of claim 81, wherein said parent protein comprises Pyrococcus furious rubredoxin (SEQ ID NO: 31) and said at least one portion of said primary sequence includes any combination of:
(i) a segment comprising isoleucine 11 of SEQ ID NO: 31;
(ii) a segment comprising glycine 17 through glycine 22 of SEQ ID NO: 31;
(iii) a segment comprising proline 33 through aspartic acid 35 of SEQ ID NO: 31;
(iv) a segment comprising valine 37 of SEQ ID NO: 31; and
(v) a segment comprising glycine 42 through serine 46 of SEQ ID NO: 31.

97. The engineered protein of claim 81, wherein said parent protein comprises rubredoxin and said engineered protein has the sequence:
MAKWVCKICGYIYDEDAG(Z)₁ISPGTKFEEL(Z)₂WTCPIC(Z)₃FEKLED (SEQ ID NO: 37)
wherein (Z)₁, (Z)₂, and (Z)₃ are each a portion in said at least one portion of the primary sequence of said engineered protein that is determined by the operation of the engineering scheme on the primary sequence of the parent protein.

98. A composition comprising the engineered protein of claim 81 and a physiologically-acceptable carrier.

99. The engineered protein of claim 81, wherein said at least one portion of the primary sequence of said engineered protein collectively is less than twenty percent of the total sequence of said engineered protein.

100. The engineered protein of claim 81, wherein said operation of said engineering scheme results in an increase or decrease in the overall number of residues present in the engineered protein relative to the number of residues present in the parent protein.

101. The engineered protein of claim 81, wherein said at least one portion of the primary sequence of said engineered protein that is determined by an engineering scheme contains one or more amino acid residue positions that are identical to the corresponding residues in the parent protein.

102. The engineered protein of claim 81, wherein said engineered protein has an N-terminus and a C-terminus, and wherein said N-terminus or said C-terminus, or both, is attached to an affinity tag.

103. The engineered protein of claim 81, wherein the N-terminal portion of said engineered protein includes a serine residue or a threonine residue and said engineered protein is attached to a surface by selectively oxidizing said serine residue or said threonine residue to form a glyoxylyl group or a keto group that is then reacted with a functionality on said surface.

104. The engineered protein of claim 103, wherein said functionality is an aminooxy or a hydrazine functionality.

105. The engineered protein of claim 103, wherein said functionality is provided by a heterobifunctional compound, said heterobifunctional compound bearing both an aminooxy- or a hydrazine-functionality and a second reactive group that attaches to said surface.

106. The engineered protein of claim 81, wherein the N-terminal portion of said engineered protein includes a cysteine residue and said engineered protein is attached to a surface by selectively derivatizing said cysteine reside by reacting it with a thioester functionality on said surface.

107. The engineered protein of claim 106, wherein said thioester functionality is provided by a heterobifunctional compound, said heterobifunctional compound bearing both a thioester functionality and a second reactive group that attaches to said surface.

108. A nucleic acid encoding the engineered protein of claim 81.

109. The nucleic acid of claim 108, wherein said nucleic acid is DNA.

110. The nucleic acid of claim 108, comprising a nucleotide sequence that hybridizes under conditions of high stringency to SEQ ID NO: 34 or the complement of SEQ ID NO: 34.

111. The nucleic acid of claim 108, comprising a nucleotide sequence that hybridizes under conditions of moderate stringency to SEQ ID NO: 34 or a nucleotide sequence that hybridizes under conditions of moderate stringency to the complement of SEQ ID NO: 34.

112. The nucleic acid of claim 108, comprising a nucleotide sequence that is at least 65% identical to SEQ ID NO: 34 or is at least 65% identical to the complement of SEQ ID NO: 34.

113. The nucleic acid of claim 108, comprising a nucleotide sequence that is at least 80% identical to SEQ ID NO: 34 or is at least 80% identical to the complement of SEQ ID NO: 34.

114. The nucleic acid of claim 108, comprising a nucleotide sequence that is at least 90% identical to SEQ ID NO: 34 or is at least 90% identical to the complement of SEQ ID NO: 34.

115. An expression vector comprising the nucleic acid of claim 108.

116. A host cell comprising the nucleic acid of claim 108.

117. An array comprising a plurality of engineered proteins immobilized on a solid support, wherein each engineered protein in the array of engineered proteins corresponds to a parent protein that has a zinc-bound fold or an iron-bound fold and wherein the primary sequence of the parent protein includes two CXₙC motifs, wherein X is a residue of any naturally occurring amino acid and n is 1, 2, 3, or 4; and
wherein at least one portion of the primary sequence of each said engineered protein in said plurality of engineered proteins is determined by an operation of an engineering scheme on the primary sequence of said corresponding parent protein, with the provisos that:
(i) said at least one portion of the primary sequence of each said engineered protein in said plurality of engineered proteins that is determined by the operation of the engineering scheme on the primary sequence of the corresponding parent protein does not exceed fifty percent of the length of the primary sequence of the engineered protein; and
(ii) said at least one portion of the primary sequence of each said engineered protein in said plurality of engineered proteins that is determined by the operation of the engineering scheme on the primary sequence of the corresponding parent protein comprises at least five percent of the length of the primary sequence of the engineered protein.

118. The array of claim 117, wherein said parent protein comprises rubredoxin from Pyrococcus furiousus, Desulfovibrio gigas, Pseudomonas oleovorans, Clostridium pasteurianum, Desulfovibrio vulgaris, Desulfovibrio desulfuricans, or Guillardia theta

119. The array of claim 117, wherein at least one engineered protein in said array of engineered proteins is characterized by an ability to bind to a compound that the parent protein does not bind.

120. The array of claim 119, wherein said compound is a protein, a hormone, a low molecular weight compound, a peptide, or an oligonucleotide.

121. The array of claim 117, wherein said parent protein comprises Pyrococcus furious rubredoxin (SEQ ID NO: 31) and said at least one portion of said primary sequence includes any combination of:
(i) a segment comprising isoleucine 11 of SEQ ID NO: 31;
(ii) a segment comprising glycine 17 through glycine 22 of SEQ ID NO: 31;
(iii) a segment comprising proline 33 through aspartic acid 35 of SEQ ID NO: 31;
(iv) a segment comprising valine 37 of SEQ ID NO: 31; and
(v) a segment comprising glycine 42 through serine 46 of SEQ ID NO: 31.

122. The array of claim 117, wherein said parent protein comprises rubredoxin and each said engineered protein in said plurality of engineered proteins has a primary sequence: p1 MAKWVCKICGYIYDEDAG(Z)₁ISPGTKFEEL(Z)₂WTCPIC(Z)₃FEKLED (SEQ ID NO: 37)
wherein (Z)₁, (Z)₂, and (Z)₃ are each a portion in said at least one portion of the primary sequence of each said engineered protein in said plurality of proteins that is determined by the operation of the engineering scheme on the primary sequence of the parent protein.

123. The array of claim 117, wherein said solid support is a bead, a slide or chip.

124. A method of determining whether an engineered protein binds to a compound, wherein the parent protein that corresponds to said engineered protein has a zinc-bound fold or an iron-bound fold and the primary sequence of the parent protein includes two CXₙC motifs, wherein X is a residue of any naturally occurring amino acid and n is 1, 2, 3, or 4, and
wherein at least one portion of the primary sequence of said engineered protein is determined by an operation of an engineering scheme on the primary sequence of said parent protein, with the provisos that:
(i) said at least one portion of the primary sequence of said engineered protein that is determined by the operation of the engineering scheme on the primary sequence of the parent protein does not exceed fifty percent of the length of the primary sequence of the engineered protein; and
(ii) said at least one portion of the primary sequence of said engineered protein that is determined by the operation of the engineering scheme on the primary sequence of the parent protein comprises at least five percent of the length of the primary sequence of the engineered protein; the method comprising contacting said engineered protein with said compound.

125. The method of claim 124, wherein said engineered protein is attached to a solid support.

126. The method of claim 124, wherein said solid support is a bead, a slide or a chip.

127. The method of claim 124, wherein said engineered protein forms a complex with said compound and wherein an EC₅₀ of said complex is less than 10⁻⁶ moles/liter.

128. A method for using an engineered protein, the method comprising:
(a) contacting a compound with an array of candidate engineered proteins immobilized on a solid support, the array of engineered proteins immobilized on the solid support including said engineered protein, each said engineered protein in said array of engineered proteins comprising an engineered rubredoxin,
wherein at least one portion of the primary sequence of said engineered rubredoxin is determined by an engineering scheme, with the provisos that
(i) said at least one portion of the primary sequence of said engineered rubredoxin is greater than five percent of the primary sequence of said engineered rubredoxin; and
(ii) said at least one portion of the primary sequence of said engineered rubredoxin is less than fifty percent of the primary sequence of said engineered rubredoxin; and
(b) determining whether said engineered protein binds to said compound.

129. The method of claim 128, said method further comprising the steps of:
(c) further engineering said engineered protein that binds to said compound in step (b);
(d) forming an array on a solid support with the further engineered proteins of step (c); and
(e) repeating step (a) and step (b) using, in step (a), the array of further engineered proteins as said array of candidate engineered proteins.

130. A method for detecting a compound in a sample, the method comprising contacting said sample with an engineered protein that binds to the compound, wherein
the parent protein that corresponds to said engineered protein has a zinc-bound fold or an iron-bound fold and the primary sequence of the parent protein includes two CXₙC motifs, wherein X is a residue of any naturally occurring amino acid and n is 1, 2, 3, or 4, and wherein
at least one portion of the primary sequence of said engineered protein is determined by an operation of an engineering scheme on the primary sequence of said parent protein, with the provisos that:
(i) said at least one portion of the primary sequence of said engineered protein that is determined by the operation of the engineering scheme on the primary sequence of the parent protein does not exceed fifty percent of the length of the primary sequence of the engineered protein; and
(ii) said at least one portion of the primary sequence of said engineered protein that is determined by the operation of the engineering scheme on the primary sequence of the parent protein comprises at least five percent of the length of the primary sequence of the engineered protein.

131. The method of claim 130, the method further comprising detecting a complex between said engineered protein and said compound.

132. The method of claim 130, wherein said parent domain comprises rubredoxin.

133. The method of claim 130, wherein said engineered protein is immobilized on a bead, a slide or a chip.

134. The method of claim 130, wherein said engineered protein is immobilized on said solid support as part of an array of engineered proteins.

135. The method of claim 130, wherein said compound is a protein.

136. The method of claim 130, wherein the parent protein comprises Pyrococcus furious rubredoxin (SEQ ID NO: 31) and said at least one portion of said primary sequence includes any combination of:
(i) a segment comprising isoleucine 11 of SEQ ID NO: 31;
(ii) a segment comprising glycine 17 through glycine 22 of SEQ ID NO: 31;
(iii) a segment comprising proline 33 through aspartic acid 35 of SEQ ID NO: 31;
(iv) a segment comprising valine 37 of SEQ ID NO: 31; and
(v) a segment comprising glycine 42 through serine 46 of SEQ ID NO: 31.

137. The array of claim 130, wherein said parent protein comprises rubredoxin and said engineered protein has a primary sequence:
MAKWVCKICGYIYDEDAG(Z)₁ISPGTKFEEL(Z)₂WTCPIC(Z)₃FEKLED (SEQ ID NO: 37)
wherein (Z)₁, (Z)₂, and (Z)₃ are each a portion in said at least one portion of the primary sequence of said engineered protein that is determined by the operation of the engineering scheme on the primary sequence of the parent protein.

138. The method of claim 130, wherein a complex between said engineered protein and the compound is detected by spectroscopy, radiography, fluorescence detection, mass spectrometry, luminescence, or surface plasmon resonance.

139. The method of claim 138, wherein the EC₅₀ of the complex is less than 10⁻⁶ moles/liter.

140. A mutated rubredoxin protein, wherein one or more portions of the mutated rubredoxin protein vary by engineering of at least ten amino acids from the corresponding portion of the wild-type rubredoxin sequence and wherein the sequence of the mutated rubredoxin protein has at least 50% total amino acid sequence identity to the wild-type rubredoxin sequence.

141. The mutated rubredoxin protein of claim 140, wherein the mutated rubredoxin protein is capable of binding to a compound to form a complex, comprising the mutated rubredoxin protein and the compound, that has an EC₅₀ that is less than 10⁻⁶ moles/liter.

142. A nucleic acid molecule encoding the mutated rubredoxin protein of claim 140.

143. An expression vector comprising an expression cassette operably linked to the nucleic acid molecule of claim 142.

144. A host cell comprising the expression vector of claim 143.

145. A method of preparing an engineered rubredoxin library from a set of paired oligonucleotides, wherein the first oligonucleotide in each pair of oligonucleotides includes a region that is complementary to the corresponding second oligonucleotide in each pair of oligonucleotides, and wherein at least one oligonucleotide in the set of paired oligonucleotides includes a randomized sequence, the method comprising:
(a) mixing together, in a different reaction, each pair of paired oligonucleotides in the set of oligonucleotides and performing mutually primed DNA synthesis using a DNA polymerase;
(b) mixing the reaction products of step (a) and performing multiple cycles of denaturation, annealing, and DNA synthesis using a DNA polymarase; and
(c) amplifying the DNA constructs from step (b) encoding full-length rubredoxin domain library members; and
(d) cloning the product of step (c) into an expression vector.

146. A library of proteins that comprises a plurality of engineered proteins, wherein the parent protein that corresponds to each engineered protein in said plurality of engineered proteins has a zinc-bound fold or an iron-bound fold and the primary sequence of the parent protein includes two CXₙC motifs, wherein X is a residue of any naturally occurring amino acid and n is 1, 2, 3, or 4, and
wherein at least one portion of the primary sequence of each engineered protein in said plurality of engineered proteins is determined by an operation of an engineering scheme on the primary sequence of said parent protein, with the provisos that:
(i) said at least one portion of the primary sequence of said engineered protein that is determined by the operation of the engineering scheme does not exceed fifty percent of the length of the primary sequence of the engineered protein; and
(ii) said at least one portion of the primary sequence of said engineered protein that is determined by the operation of the engineering scheme comprises at least five percent of the length of the primary sequence of the engineered protein.

147. The library of claim 146, wherein the parent protein is in the rubredoxin-superfamily.

148. The library of claim 146, wherein the parent protein is in the rubredoxin family, the desulforedoxin family, or the cytochrome c oxidase subunit F family.

149. The library of claim 146, wherein the parent protein comprises Pyrococcus furious rubredoxin (SEQ ID NO: 31) and wherein each said at least one portion of the primary sequence of each engineered protein in said library of engineered proteins is selected from the group consisting of:
(i) a segment comprising isoleucine 11 of SEQ ID NO: 31;
(ii) a segment comprising glycine 17 through glycine 22 of SEQ ID NO: 31;
(iii) a segment comprising proline 33 through aspartic acid 35 of SEQ ID NO: 31;
(iv) a segment comprising valine 37 of SEQ ID NO: 31; and
(v) a segment comprising glycine 42 through serine 46 of SEQ ID NO: 31.

150. The library of claim 146, wherein said parent protein comprises rubredoxin and each said engineered protein in said plurality of engineered proteins has a primary sequence:
MAKWVCKICGYIYDEDAG(Z)₁ISPGTKFEEL(Z)₂WTCPIC(Z)₃FEKLED (SEQ ID NO: 37)
wherein (Z)₁, (Z)₂, and (Z)₃ are each a portion in said at least one portion of the primary sequence of each said engineered protein in said plurality of proteins that is determined by the operation of the engineering scheme on the primary sequence of the parent protein.

151. The library of claim 146, wherein each of said engineered proteins in said plurality of engineered proteins is attached to a genetically replicable package.

152. The library of claim 146, wherein the genetically replicable package is a bacteriophage.

153. The library of claim 146, wherein the bacteriophage is T7, SPbc2, SPP1, phiX174, IEM, T4, UrLamda, P22, M13, f1, P1, MS2, SPO1, B3, HK97, fXo, or λ.

154. A method of making an engineered protein, the method comprising subjecting at least one portion of the primary sequence of a parent protein to an engineering scheme in order to produce said engineered protein, with the provisos that:
(i) said parent protein has a zinc-bound fold or an iron-bound fold and the primary sequence of the parent protein includes two CXₙC motifs, wherein X is a residue of any naturally occurring amino acid and n is 1, 2, 3, or 4;
(ii) said at least one portion of the primary sequence of said engineered protein does not exceed fifty percent of the length of the primary sequence of said engineered protein; and
(iii) said at least one portion of the primary sequence of said engineered protein comprises at least five percent of the length of the primary sequence of said engineered protein.

155. The method of claim 154, wherein said engineering scheme is a pseudo-randomization scheme and the step of subjecting said at least one portion of the primary sequence of said parent protein to an engineering scheme results in the randomization of said at least one portion of the primary sequence.

156. The method of claim 154, wherein said engineering scheme is a randomization scheme and the step of subjecting said at least one portion of the primary sequence of said parent protein to an engineering scheme results in the pseudo-randomization of said at least one portion of the primary sequence.


SEQUENCE LISTING (46 sequences)

SEQ ID NO: 1 - protein, 545 aa, Thermoplasma acidophilum
MMTGQVPILVLKEGTQREQGKNAQRNNIEAAKAIADAVRTTLGPKGMDKMLVDSIGDIIISNDGATILKEMDVEHPTAKMIVEVSKAQDTAVGDGTTTAVVLSGELLKQAETLLDQGVHPTVISNGYRLAVNEARKIIDEIAEKSTDDATLRKIALTALSGKNTGLSNDFLADLVVKAVNAVAEVRDGKTIVDTANIKVDKKNGGSVNDTQFISGIVIDKEKVHSKMPDVVKNAKIALIDSALEIKKTEIEAKVQISDPSKIQDFLNQETNTFKQMVEKIKKSGANVVLCQKGIDDVAQHYLAKEGIYAVRRVKKSDMEKLAKATGAKIVTDLDDLTPSVLGEAETVEERKIGDDRMTFVMGCKNPKAVSILIRGGTDHVVSEVERALNDAIRVVAITKEDGKFLWGGGAVEAELAMRLAKYANSVGGREQLAIEAFAKALEIIPRTLAENAGIDPINTLIKLKAEHEKGRISVGVDLDNNGVGDMKAKGVVDPLRVKTHALESAVEVATMILRIDDVIASKKSTPPSGQGGQGQGMPGGGMPEY

SEQ ID NO: 2 - DNA, 1800 bp, Thermoplasma acidophilum
ttgatagaaatgtcatgactccagttggatatcccatattccggcattttgtcatgccagtgacttcagcaaagtttatattgaagaacatatttatccgtctgctaggtgataagcaatatgatgacgggacaggttccaattctagttctgaaagaaggcacacagagagagcagggtaaaaacgcacagaggaataatatagaggcagcaaaggcgattgccgatgccgtcaggaccacacttggcccaaagggaatggacaagatgctggtcgattccattggcgatataataatttcaaatgatggtgcaacgatcctcaaggagatggatgtggagcatccaacggcgaagatgatcgtagaggtctccaaggcgcaggataccgccgtgggtgatggtacaacgactgctgttgtgctctctggcgagttgctcaagcaggctgagacactgctcgatcagggtgtgcatccaaccgtcatatccaacggatacaggctcgcggtaaatgaggcaagaaagatcatagacgaaatagctgagaaatcaacagacgacgcaaccttgaggaagatagctctcaccgcactgtcagggaagaacacaggtttgtcaaacgacttcctggctgatcttgtcgttaaggcggtcaatgcagttgctgaggtcagagatggaaagacgatagttgacacagccaacataaaggtggacaagaaaaacggcggaagtgtcaatgacactcagttcataagcggtatcgtcatagacaaggaaaaggttcattccaagatgcctgatgtggtcaagaacgcaaagatcgctctgatcgattccgcgcttgagataaagaagacggaaattgaagccaaggtccagatatcggatccaagcaagatacaggatttccttaaccaagagacgaacacgttcaagcagatggtcgagaagataaagaagagcggtgcgaatgtcgtcctctgccagaagggcatcgatgatgtggcgcagcactaccttgccaaggaaggcatatacgctgttcgcagagtcaagaagagcgatatggagaagttggccaaggccacaggtgctaagatagttacggatcttgacgatcttactccatccgtcctaggcgaggcggaaaccgttgaggagcgcaagatcggcgatgacagaatgaccttcgtcatgggatgcaagaatccaaaggcggtcagcatactgatcagaggaggaacagaccacgttgtctcggaggtcgagagggcgcttaacgatgctataagggtcgttgctataacaaaagaggatggaaaattcctctggggcggaggagcagttgaggccgaactggcaatgaggcttgccaaatacgcgaatagcgtcggtggtagggagcagctggctatagaagcctttgcgaaggccctggagataattccgaggacgctggctgaaaatgcgggaattgatccgatcaacaccctgataaagctcaaggcggagcatgaaaaaggacgcatatccgtcggagtggatctcgataacaacggtgtcggggacatgaaggcaaagggcgtcgtagatcctcttagggtcaaaacacacgcgctcgagagcgccgtcgaggtcgcaacaatgatactgagaatcgacgatgtaatcgcaagcaagaagtccacgccgccttccggacagggtggccagggacagggaatgccaggcggcggaatgcctgagtactgaaaatttttccatcgtccctccttttttatttcttttttttgc

SEQ ID NO: 3 - DNA, 522 bp, Thermoplasma acidophilum
  misc_feature (1)..(522): n = any nucleotide a, t, g, or c
tctggtatcgttatcnnnnnnnnnnnnnnnnnnnnnnnnatgccgbdygtagttaagaacgctaagatagcgctgatcgactctgctctggaaatcaaaaaaaccgaaatcgaagctaaagttcagatctctgacccgtctaaaatccaggacttcctgaaccaggaaaccaacaccttcaaacagatggtagaaaaaattaaaaaatctggtgctaacgtagttctgtgcnnnnnnnnnnnnnnnnnngttgctnnnnnntacctagccaaggaaggtatctacgctgttnnnnnngttnnnnnntctgacatggaaaaactagctaaagctaccggtgctaaaatcgttaccgacctggacgacctgaccccgtctgttctaggtgaagctgaaaccgtagaagaacgtnnnnnnnnnnnnnnnnnnnnnacctacgttatgggttgtaaaggctctgtaagccatcatcaccaccatcactctgaacagaaactgatctctgaagaagacctgctggcc

SEQ ID NO: 4 - DNA, 36 bp, Artificial Sequence
  Primer used to create randomized loop in the substrate-binding domain of the alpha subunit of Thermoplasma acidophilum
gatgacgacaggatccccgcttctggtatcgttatc

SEQ ID NO: 5 - DNA, 73 bp, Artificial Sequence
  Primer used to create randomized loop in the substrate-binding domain of the alpha subunit of Thermoplasma acidophilum
ggcgaagaccatagcaatagnnnnnnnnnnnnnnnnnnnnnnnntacggcnnncatcaattcttgcgattcta

SEQ ID NO: 6 - DNA, 30 bp, Artificial Sequence
  Primer used to create randomized loop in the substrate-binding domain of the alpha subunit of Thermoplasma acidophilum
gtagttaagaacgctaagatagcgctgatc

SEQ ID NO: 7 - DNA, 45 bp, Artificial Sequence
  Primer used to create randomized loop in the substrate-binding domain of the alpha subunit of Thermoplasma acidophilum
tgcgattctatcgcgactagctgagacgagacctttagttttttt

SEQ ID NO: 8 - DNA, 50 bp, Artificial Sequence
  Primer used to create randomized loop in the substrate-binding domain of the alpha subunit of Thermoplasma acidophilum
gactctgctctggaaatcaaaaaaaccgaaatcgaagctaaagttcagat

SEQ ID NO: 9 - DNA, 51 bp, Artificial Sequence
  Primer used to create randomized loop in the substrate-binding domain of the alpha subunit of Thermoplasma acidophilum
ggctttagcttcgatttcaagtctagagactgggcagattttaggtcctga

SEQ ID NO: 10 - DNA, 52 bp, Artificial Sequence
  Primer used to create randomized loop in the substrate-binding domain of the alpha subunit of Thermoplasma acidophilum
ctctgacccgtctaaaatccaggacttcctgaaccaggaaaccaacaccttc

SEQ ID NO: 11 - DNA, 50 bp, Artificial Sequence
  Primer used to create randomized loop in the substrate-binding domain of the alpha subunit of Thermoplasma acidophilum
aggacttggtcctttggttgtggaagtttgtctaccatcttttttaattt

SEQ ID NO: 12 - DNA, 51 bp, Artificial Sequence
  Primer used to create randomized loop in the substrate-binding domain of the alpha subunit of Thermoplasma acidophilum
aaacagatggtagaaaaaattaaaaaatctggtgctaacgtagttctgtgc

SEQ ID NO: 13 - DNA, 71 bp, Artificial Sequence
  Primer used to create randomized loop in the substrate-binding domain of the alpha subunit of Thermoplasm acidophilum
ccacgattgcatcaagacacgnnnnnnnnnnnnnnnnnncaacgannnnnnatggatcggttccttccata

SEQ ID NO: 14 - DNA, 30 bp, Artificial Sequence
  Primer used to create randomized loop in the substrate-binding domain of the alpha subunit of Thermoplasma acidophilum
tacctagccaaggaaggtatctacgctgtt

SEQ ID NO: 15 - DNA, 56 bp, Artificial Sequence
  Primer used to create randomized loop in the substrate-binding domain of the alpha subunit of Thermoplasma acidophilum
ttccttccatagatgcgacaannnnnncaannnnnnagactgtacctttttgatcg

SEQ ID NO: 16 - DNA, 45 bp, Artificial Sequence
  Primer used to create randomized loop in the substrate-binding domain of the alpha subunit of Thermoplasma acidophilum
tctgacatggaaaaactagctaaagctaccggtgctaaaatcgtt

SEQ ID NO: 17 - DNA, 55 bp, Artificial Sequence
  Primer used to create randomized loop in the substrate-binding domain of the alpha subunit of Thermoplasma acidophilum
gatggccacgattttagcaatggctggacctgctggactggggcagacaagatcc

SEQ ID NO: 18 - DNA, 45 bp, Artificial Sequence
  Primer used to create randomized loop in the substrate-binding domain of the alpha subunit of Thermoplasma acidophilum
ctgaccccgtctgttctaggtgaagctgaaaccgtagaagaacgt

SEQ ID NO: 19 - DNA, 63 bp, Artificial Sequence
  Primer used to create randomized loop in the substrate-binding domain of the alpha subunit of Thermoplasma acidophilum
tcgactttggcatcttcttgcannnnnnnnnnnnnnnnnnnnntggatgcaatacccaacatt

SEQ ID NO: 20 - DNA, 41 bp, Artificial Sequence
  Primer used to create randomized loop in the substrate-binding domain of the alpha subunit of Thermoplasma acidophilum
acctacgttatgggttgtaaaggctctgtaagccatcatca

SEQ ID NO: 21 - DNA, 45 bp, Artificial Sequence
  Primer used to create randomized loop in the substrate-binding domain of the alpha subunit of Thermoplasma acidophilum
tccgagacattcggtagtagtggtggtagtgagacttgtctttga

SEQ ID NO: 22 - DNA, 46 bp, Artificial Sequence
  Primer used to create randomized loop in the substrate-binding domain of the alpha subunit of Thermoplasma acidophilum
ccaccatcactctgaacagaaactgatctctgaagaagacctgctg

SEQ ID NO: 23 - DNA, 38 bp, Artificial Sequence
  Primer used to create randomized loop in the substrate-binding domain of the alpha subunit of Thermoplasma acidophilum
ctagagacttcttctggacgacgcagatctcatcctgc

SEQ ID NO: 24 - protein, 543 aa, Thermoplasma acidophilum
MIAGQPIFILKEGTKRESGKDAMKENIEAAIAISNSVRSSLGPRGMDKMLVDSLGDIVITNDGVTILKEMDVEHPAAKMMVEVSKTQDSFVGDGTTTAVIIAGGLLQQAQGLINQNVHPTVISEGYRMASEEAKRVIDEISTKIGADEKALLLKMAQTSLNSKSASVAKDKLAEISYEAVKSVAELRDGKYYVDFDNIQVVKKQGGAIDDTQLINGIIVDKEKVHPGMPDVVKDAKIALLDAPLEIKKPEFDTNLRIEDPSMIQKFLAQEENMLREMVDKIKSVGANVVITQKGIDDMAQHYLSRAGIYAVRRVKKSDMDKLAKATGASIVSTIDEISSSDLGTAERVEQVKVGEDYMTFVTGCKNPKAVSILVRGETEHVVDEMERSITDSLHVVASALEDGAYAAGGGATAAEIAFRLRSYAQKIGGRQQLAIEKFADAIEEIPRALAENAGLDPIDILLKLRAEHAKGNKTYGINVFTGEIEDMVKNGVIEPIRVGKQAIESATEAAIMILRIDDVIATKSSSSSSNPPKSGSSSESSED

SEQ ID NO: 25 - DNA, 556 bp, Artificial Sequence
  Product of assembly reaction
gatgacgacaggatccccgcttctggtatcgttatcnnnnnnnnnnnnnnnnnnnnnnnnatgccgnnngtagttaagaacgctaagatagcgctgatcgactctgctctggaaatcaaaaaaaccgaaatcgaagctaaagttcagatctctgacccgtctaaaatccaggacttcctgaaccaggaaaccaacaccttcaaacagatggtagaaaaaattaaaaaatctggtgctaacgtagttctgtgcnnnnnnnnnnnnnnnnnngttgctnnnnnntacctagccaaggaaggtatctacgctgttnnnnnngttnnnnnntctgacatggaaaaactagctaaagctaccggtgctaaaatcgttaccgacctggacgacctgaccccgtctgttctaggtgaagctgaaaccgtagaagaacgtnnnnnnnnnnnnnnnnnnnnnacctacgttatgggttgtaaaggctctgtaagccatcatcaccaccatcactctgaacagaaactgatctctgaagaagacctgctgcgtctagagtaggacg

SEQ ID NO: 26 - protein, 183 aa, Artificial Sequence
  Expected assembly reaction product
DDDRIPASGIVIXXXXXXXXMPXVVKNAKIALIDSALEIKKTEIEAKVQISDPSKIQDFLNQETNTFKQMVEKIKKSGANVVLCXXXXXXVAXXYLAKEGIYAVXXVXXSDMEKLAKATGAKIVTDLDDLTPSVLGEAETVEERXXXXXXXTYVMGCKGSVSHHHHHHSEQKLISEEDLLRLE

SEQ ID NO: 27 - DNA, 30 bp, Artificial Sequence
  Primer used in amplification reaction
gctacgaattccgcttctggtatcgttatc

SEQ ID NO: 28 - DNA, 22 bp, Artificial Sequence
  Primer used in amplification reaction
agataccttccttggctaggta

SEQ ID NO: 29 - DNA, 22 bp, Artificial Sequence
  Primer used in amplification reaction
tacctagccaaggaaggtatct

SEQ ID NO: 30 - DNA, 35 bp, Artificial Sequence
  Primer used in amplification reaction
agcaggataagcttaggccagcaggtcttcttcag

SEQ ID NO: 31 - protein, 53 aa, Pyrococcus furiosus
AKWVCKICGYIYDEDAGDPDNGISPGTKFEELPDDWVCPICGAPKSEFEKLED

SEQ ID NO: 32 - protein, 70 aa, Pyrococcus furiosus
GSSGSGMAKWVCKICGYIYDEDAGDPDNGISPGTKFEELPDDWVCPICGAPKSEFEKLEDGGSGHHHHHH

SEQ ID NO: 33 - protein, 76 aa, Pyrococcus furiosus
  MISC_FEATURE (1)..(76): Xaa = any amino acid
GSSGSGMAKWVCKICGYIYDEDAGXXXXXXXXXISPGTKFEELXXXWXCPICXXXXXXXXFEKLEDGGSGHHHHHH

SEQ ID NO: 34 - DNA, 189 bp, Pyrococcus furiosus
atggctaaatgggtttgcaaaatctgcggttacatctacgacgaagacgctggtgacccggacaacggtatctctccgggtaccaaattcgaagaactgccggacgactgggtttgcccgatctgcggtgctccgaaatctgaattcgaaaaactggaagacggttctcaccaccaccaccaccactag

SEQ ID NO: 35 - DNA, 282 bp, Artificial Sequence
  Engineered rubredoxin library
gctacgaattcctctggcagcggcatggcgaaatgggtttgcaaaatctgcggttacatttacgacgaagacgcgggcnnnnnnnnnnnnnnnatctcaccaggtaccaaattcgaagagctgnnnnnnnnntggacgtgtccgatctgcnnnnnnnnnnnnnnnnnnttcgagaaactggaggatgcgggctctggtggtcaccaccatcaccatcactctggttcctctgaacagaaactgatctctgaagaggatctgctggcctaagcttatcctgct

SEQ ID NO: 36 - DNA, 282 bp, Artificial Sequence
  Engineered rubredoxin library
cgatgcttaaggagaccgtcgccgtaccgctttacccaaacgttttagacgccaatgtaaatgctgcttctgcgcccgnnnnnnnnnnnnnnntagagtggtccatggtttaagcttctcgacnnnnnnnnnacctgcacaggctagacgnnnnnnnnnnnnnnnnnnaagctctttgacctcctacgcccgagaccaccagtggtggtagtggtagtgagaccaaggagacttgtctttgactagagacttctcctagacgaccggattcgaataggacga

SEQ ID NO: 37 - protein, 18 aa, Artificial Sequence
  Engineered rubredoxin protein
MAKWVCKICGYIYDEDAG

SEQ ID NO: 38 - protein, 61 aa, Artificial Sequence
  Engineered rubredoxin protein
MAKWVCKICGYIYDEDAGFVEYYGHPNISPGTKFEELSRYVDGWTCPICSYGMPIFEKLED

SEQ ID NO: 39 - protein, 63 aa, Artificial Sequence
  Engineered rubredoxin protein
MAKWVCKICGYIYDEDAGEDPGHRSRYISPGTKFEELTTGWTCPICRCTNSTTSTNCFEKLED

SEQ ID NO: 40 - protein, 58 aa, Artificial Sequence
  Engineered rubredoxin protein
MAKWVCKICGYIYDEDAGFVEYYDDGVISPGTKFEELHTAWTCPICSCTYNPFEKLED

SEQ ID NO: 41 - protein, 183 aa, Artificial Sequence
  Engineered rubredoxin protein
DDDRIPASGIVIDRVGRDSNMPHVVKNAKIALIDSALEIKKTEIEAKVQISDPSKIQDFLNQETNTFKQMVEKIKKSGANVVLCADDYAHVAYDYLAKEGIYAVGYVTESDMEKLAKATGAKIVTDLDDLTPSVLGEAETVEERPWGNNKETYVMGCKGSVSHHHHHHSEQKLISEEDLLRLE

SEQ ID NO: 42 - protein, 183 aa, Artificial Sequence
  Engineered rubredoxin protein
DDDRIPASGIVIVGHNKVPSMPRVVKNAKIALIDSALEIKKTEIEAKVQISDPSKIQDFLNQETNTFKQMVEKIKKSGANVVLCGYKNLTVAYEYLAKEGIYAVYHVDESDMEKLAKATGAKIVTDLDDLTPSVLGEAETVEERGTANAPATYVMGCKGSVSHHHHHHSEQKLISEEDLLRLE

SEQ ID NO: 43 - protein, 183 aa, Artificial Sequence
  Engineered rubredoxin protein
DDDRIPASGIVIARPGESAFMPDVVKNAKIALIDSALEIKKTEIEAKVQISDPSKIQDFLNQETNTFKQMVEKIKKSGANVVLCNGDQADVAYDYLAKEGIYAVHFVDESDMEKLAKATGAKIVTDLDDLTPSVLGEAETVEERKYTEGVPTYVMGCKGSVSHHHHHHSEQKLISEEDLLRLE

SEQ ID NO: 44 - protein, 10 aa, Artificial Sequence
  Engineered rubredoxin protein
ISPGTKFEEL

SEQ ID NO: 45 - protein, 6 aa, Artificial Sequence
  Engineered rubredoxin protein
WTCPIC

SEQ ID NO: 46 - protein, 6 aa, Artificial Sequence
  Engineered rubredoxin protein
FEKLED